(19) United States
(12) Patent Application Publication
(10) Pub. No.: US 2024/0379228 A1
(43) Pub. Date: Nov. 14, 2024

(54) DIAGNOSIS OF COLORECTAL CANCER USING TARGETED QUANTIFICATION OF PEPTIDES

(71) Applicant: VENN BIOSCIENCES CORPORATION, South San Francisco, CA (US)

(72) Inventors: Karina ISLAS RIOS, Melbourne (AU); Daniel SERIE, San Francisco, CA (US); Khushbu Yatin DESAI, Redwood City, CA (US); Daniel Willem HOMMES, Westlake Village, CA (US); Maurice Yu WONG, San Mateo, CA (US)

(73) Assignee: VENN BIOSCIENCES CORPORATION, South San Francisco, CA (US)

(21) Appl. No.: 18/451,015

(22) Filed: Aug. 16, 2023

Related U.S. Application Data
(60) Provisional application No. 63/500,852, filed on May 8, 2023.
(60) Provisional application No. 63/505,954, filed on Jun. 2, 2023.

(51) Int. Cl. G16H 50/20; C07K 14/47; G01N 33/68; G16B 15/20; G16B 35/20; G16B 40/20

(52) U.S. Cl. CPC G16H 50/20; C07K 14/473; G01N 33/6848; G16B 15/20; G16B 35/20; G16B 40/20; G01N 2333/4728

(57) ABSTRACT

The present disclosure encompasses systems, methods, and compositions for diagnosing a subject for an AA or colorectal cancer (CRC) disease state by ascertaining the presence of certain one or more glycosylated or aglycosylated peptides in liquid biopsy samples from the subject. Specific embodiments encompass methods of measuring certain one or more glycosylated or aglycosylated peptides in liquid biopsy samples from subjects known to have or suspected of having an AA or CRC disease state or subjects undergoing routine health care maintenance for possible presence of an AA or CRC disease state. The disclosure provides systems, methods, and compositions to identify subjects at-risk for CRC or AA and increases subject colonoscopy compliance, in specific embodiments.

### CROSS-REFERENCE TO RELATED APPLICATIONS

This application claims the priority benefit of U.S. Provisional Patent Application Ser. 63/500,852, filed May 8, 2023, [Attorney Docket No. VENN-00059PR]; and U.S. Provisional Patent Application Ser. No. 63/505,954, filed Jun. 2, 2023, [Attorney Docket VENN-00059P1]; which are hereby all incorporated by reference herein in their entirety.

### FIELD

The present disclosure generally relates to methods and systems for analyzing peptide structures for diagnosing and/or treating adenomas or colorectal cancer. More particularly, the present disclosure relates to analyzing quantification data for a set of peptide structures detected in a biological sample obtained from a subject for use in diagnosing and/or treating the subject, the set of peptide structures being associated with adenomas or colorectal cancer.

### SEQUENCE LISTING

The instant application contains a Sequence Listing which has been submitted via Patent Center and is hereby incorporated by reference in its entirety. Said .xml copy, created on Sep. 4, 2023 is named VENN-00059US, and is 166.7 KG in size.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat colorectal cancer.

Colorectal cancers (CRCs) typically develop from colon adenomas, among which “advanced” colon adenomas are considered to be the clinically relevant precursors of CRCs. A colon adenoma is a type of polyp, or unusual growth of cells that form a small clump (i.e., colon mass or tumor) in the lining of the colon that is not cancer. While most of them are benign, or not dangerous, up to 10 percent of advanced colon adenomas can transform into cancer. Finding CRCs and/or advanced adenomas early can lead to better survival statistics for patients. Most CRCs and advanced adenomas are currently diagnosed using more invasive diagnostic techniques such as a colonoscopy and/or a tissue biopsy. Since many patients delay or are reluctant to undergo invasive-type diagnostic procedures, it is important to develop less invasive or non-invasive diagnostic methods that are able to identify patients who have colon masses of concern and classify those masses as CRCs (i.e., malignant) or advanced adenomas (i.e., non-malignant) so that they can be properly treated.

Thus, an approach that is non-invasive, accurate, and reliable and that enables early diagnosis is needed. An approach enabling early diagnosis may help reduce negative health outcomes in patients with colorectal cancer and/or increase the effectiveness of preventative treatment of precursors (i.e., advanced adenomas) to colorectal cancer. Such an approach can assist in guiding a patient to an urgency for further testing, for example, including a colonoscopy procedure. Thus, it may be desirable to have methods and systems capable of addressing one or more of the above-identified issues.

### SUMMARY

Embodiments of the disclosure encompass systems, methods, and compositions related to diagnosing a subject for an adenoma or colorectal cancer (CRC) disease state by ascertaining the presence of certain one or more glycosylated or aglycosylated peptides in liquid biopsy samples from the subject. Specific embodiments encompass methods of measuring certain one or more glycosylated or aglycosylated peptides in liquid biopsy samples from subjects known to have or suspected of having an adenoma or CRC disease state or subjects undergoing routine health care maintenance for possible presence of an adenoma or CRC disease state. Subjects suspected of having an adenoma or CRC disease state or those undergoing routine health care maintenance may or may not have one or more symptoms of an adenoma or CRC disease state, such as anemia. abdominal pain. dark or bloody stools. Rectal bleeding, constipation or diarrhea, unexplained weight loss, and/or feeling that the bowel does not empty all the way. Subjects having the certain one or more glycosylated or aglycosylated peptides are directed for further testing, such as a colonoscopy.

In various embodiments, the present disclosure provides systems, methods, and compositions with the ability to identify subjects in need of further testing for an adenoma or CRC disease state, such as a colonoscopy, because their glycoproteomic profile indicates they are at risk for either advanced adenoma or CRC. Such embodiments allow for early detection and intervention (even at the advanced adenoma stage), leading to significantly better outcomes and survival rates for the subjects. These embodiments improve subject compliance, given the indication of a higher risk for advanced adenoma or CRC in subjects having the one or more certain glycosylated or aglycosylated peptide(s) and a need for a follow-up procedure, including a colonoscopy.

In various embodiments, a method for diagnosing a subject with respect to an advanced adenoma (AA) or colorectal cancer (CRC) disease state includes receiving peptide structure data corresponding to a biological sample obtained from the subject; analyzing the peptide structure data using at least one supervised machine learning model to generate a disease indicator that indicates whether the biological sample evidences the advanced adenoma or CRC disease state based on at least one peptide structure selected from a group of peptide structures identified in Table 2; wherein the group of peptide structures in Table 2 is associated with the advanced adenoma or CRC disease state; and generating a diagnosis output based on the disease indicator.

In various embodiments, a composition includes at least one peptide structure of Table 2.

In various embodiments, a composition includes a peptide structure or a product ion, wherein: the peptide structure or the product ion comprises an amino acid sequence having at least 90% sequence identity to any one of SEQ ID NOS: 3-9, 12, 14-16, 18, 25-28, and 31-35, corresponding to peptide structures in Table 2 and Table 4C; and the product ion is selected as one from a group consisting of product ions identified in Table 3B including product ions falling within an identified m/z range.

In various embodiments, a composition includes a glycopeptide structure selected as at least one peptide structure identified in Table 2, wherein: the glycopeptide structure comprises: an amino acid peptide sequence identified in Table 4C as corresponding to the glycopeptide structure; and a glycan as corresponding to the glycopeptide structure in which the glycan structure is linked to a residue of the amino acid peptide sequence at a corresponding position identified in Table 2; and wherein the glycan structure has a glycan composition.

In various embodiments, a method of screening a subject for an advanced adenoma or CRC disease state includes analyzing a peptide structure data using at least one supervised machine learning model to generate a disease indicator that indicates whether the biological sample evidences the advanced adenoma or CRC disease state based on at least one peptide structure selected from a group of peptide structures identified in Table 2, wherein peptide structure data corresponds to a biological sample obtained from the subject; and outputting either a recommendation to perform a colonoscopy or to not perform the colonoscopy based on the disease indicator.

In various embodiments, a method for diagnosing a subject with colorectal cancer (CRC) includes detecting a presence or amount of at least one peptide structure selected from a group of peptide structures identified in Table 8B in a biological sample obtained from the subject and thereby diagnosing the individual as having colorectal cancer or not having colorectal cancer based upon the presence or amount of the at least one peptide structure selected from the group of peptide structures identified in Table 8B.

In various embodiments, a method for determining a relative risk for a presence of polyps in a subject includes detecting a presence or amount of at least one peptide structure selected from a group of peptide structures identified in Table 9 in a biological sample obtained from the subject and thereby determining the relative risk for the presence of polyps in the subject based upon the presence or amount of the at least one peptide structure selected from the group of peptide structures identified in Table 9.

### BRIEF DESCRIPTION OF THE DRAWINGS

The present disclosure is described in conjunction with the appended figures.

FIG. 1 is a schematic diagram of an exemplary workflow 100 for the detection of peptide structures associated with a disease state for use in diagnosis and/or treatment in accordance with one or more embodiments.

FIG. 2A (SEQ ID NO: 103) is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5A is a flowchart of a process for diagnosing a subject with respect to a colorectal cancer disease state in accordance with one or more embodiments based on the biomarkers of Table 1.

FIG. 5B is another flowchart of a process for diagnosing a subject with respect to a colorectal cancer disease state in accordance with one or more embodiments based on the biomarkers of Table 2.

FIG. 6A is a flowchart of a process for training a model to diagnose a subject with respect to advanced adenoma or CRC disease state in accordance with one or more embodiments based on the biomarkers of Table 1.

FIG. 6B is a flowchart of a process for training a model to diagnose a subject with respect to advanced adenoma or CRC disease state in accordance with one or more embodiments based on the biomarkers of Table 2.

FIG. 7A is a flowchart of a process for monitoring a subject for an advanced adenoma or CRC in accordance with one or more embodiments based on the biomarkers of Table 1.

FIG. 7B is a flowchart of a process for monitoring a subject for an advanced adenoma or CRC in accordance with one or more embodiments based on the biomarkers of Table 2.

FIG. 8 illustrates a series of receiver operating characteristic (ROC) curves representing discriminatory performance for all possible thresholds of the developed classifier model, stratified by sample set based on the biomarkers of Table 1.

FIG. 9 is a chart illustrating the distributions of predicted probabilities in each phenotype, stratified by dataset based on the biomarkers of Table 1.

FIG. 10(a) to FIG. 10(e) are bar graph representations of the glycan fraction for fucosylated tri-antennary glycans with two sialic acids (FIG. 10(a)) and with three sialic acid (FIG. 10(b)); fucosylated tetra-antennary glycans with two sialic acids (FIG. 10(c)), three sialic acids (FIG. 10(d)), and four sialic acids (FIG. 10(e)). FIG. 10(f) is a bar graph representation of the glycan fraction for high mannose (M5-M9) glycans containing glycopeptides (*p-value<=0.05, **p-value<=0.01, ***p-value<=0.001, **** p-value<=0.0001). For each of FIG. 10(a) to FIG. 10(e), the glycan fractions were illustrated for the cohorts labeled as healthy, Non-AA, AA (without high grade dysplasia), High-RISK-AA (high grade dysplasia only), and colorectal cancer (from left to right).

FIG. 11A illustrates a series of receiver operating characteristic (ROC) curves representing discriminatory performance for all possible thresholds of the developed classifier model, stratified by sample set based on the biomarkers of Table 2.

FIG. 11B is a chart illustrating the distributions of predicted probabilities in each phenotype, stratified by dataset based on the biomarkers of Table 2.

FIG. 12 is a chart showing the association between lifestyle/concomitant factors and presence of adenomatous, serrated, and/or hyperplastic polyps. Association expressed as the relative risks of having a particular polyp type than not having a polyp. Only associations with a p-value<0.05 were illustrated.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, serum sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

However, to understand various disease conditions and to diagnose certain diseases, such as colorectal cancer, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns that may be able to provide information about a disease state (e.g., a colorectal cancer disease state). This information can be used to distinguish the disease state from other states, diagnose a subject as having or not having the disease state, determine a likelihood that a subject has the disease state, or a combination thereof. For example, such analysis may be useful in diagnosing an advanced adenoma or colorectal cancer disease state for a subject (e.g., a negative diagnosis for the advanced adenoma or colorectal cancer (and/or advanced adenoma) disease state, a positive diagnosis for the advanced adenoma or colorectal cancer disease state). Sample collection and analysis can be collected at different time points for comparing adenoma or colorectal cancer disease states over time for a subject. For example, the negative diagnosis may include a healthy state. An example of the positive diagnosis includes the subject suffering from colorectal cancer or advanced adenoma disease state. A diagnosis can also assess a malignancy status of a previously identified colorectal tumor (or mass).

Accordingly, the embodiments described herein provide various methods and systems for analyzing proteins in subjects and, in particular, glycoproteins. In one or more embodiments, one or more machine learning models are trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing an advanced adenoma or colorectal cancer disease state. An adenoma or colorectal cancer disease state may include any condition that can be diagnosed as an advanced adenoma or cancer that occurs in the colon or rectum. Certain peptide structures that are associated with an advanced adenoma or colorectal cancer disease state may be more relevant to that disease state than other peptide structures that are also associated with that disease state.

Analyzing the abundance of peptide sequences and glycosylated peptide sequences in a biological sample may provide a more accurate way in which to distinguish a positive colorectal cancer disease state (e.g., a state including the presence of colorectal cancer) from a negative colorectal cancer disease state (e.g., healthy state, an absence of colorectal cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

Further, the methods, systems, and compositions provided by the embodiments described herein may enable an earlier, more accurate and/or less invasive diagnosis of colorectal cancer in a subject as compared to currently available diagnostic modalities (e.g., colonoscopy, biopsies, imaging, biochemical tests) used for determining whether surgical intervention is indicated.

The description below provides exemplary implementations of the methods and systems described herein for the research, diagnosis, and/or treatment of a colorectal cancer disease state. Various examples implement the methods and systems described herein as a screening tool. Descriptions and examples of various terms, as used herein, are provided in Section II below.

### II. Exemplary Descriptions of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

“Treating” or treatment of a disease or condition refers to executing a protocol, which may include administering one or more drugs to an individual, such as a patient (or subject), in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including adenomas or colorectal cancer.

The term “colorectal cancer” as used herein refers to cancer that starts in the colon or the rectum.

The term “colorectal cancer (CRC) disease state” as used herein refers to the presence in an individual of colorectal cancer of any type and of any stage.

The term “early stage” as used herein refers to stage 0, stage 1, or stage 2 colorectal cancer, such as defined by the American Joint Committee on Cancer (AJCC) TNM system and based on the size of the tumor, whether or not it has spread to nearby lymph nodes, and whether or not it has spread to distant sites.

The term “late stage” as used herein refers to stage 3 or stage 4 colorectal cancer, such as defined by the American Joint Committee on Cancer (AJCC) TNM system and based on the size of the tumor, whether or not it has spread to nearby lymph nodes, and whether or not it has spread to distant sites.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refers to a biological process that employs enzymes to break specific amino acid peptide bonds. For example, digesting a peptide includes contacting the peptide with a digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refers to a peptide or polypeptide comprising at least one glycan residue. In various embodiments, glycopeptides comprise carbohydrate moieties (e.g., one or more glycans) covalently attached to a side chain (i.e., R group) of an amino acid residue.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein. A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio,” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” As used herein, the phrase “peptide,” is meant to include glycopeptides unless stated otherwise.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ(H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses). A subject may be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition. A subject may also be referred to as an individual or patient.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of linear or nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

As used herein, “Conventional Adenoma” refers to a tubular, tubulovillous or villous adenomas.

As used herein, an “Advanced Adenoma” refers to a subject containing one of (i) a polyp measuring ≥1 cm in the greatest dimension, (ii) a polyp of any size with high-grade dysplasia, (iii) a tubulovillous/villous polyp of any size, and (iv) a combination thereof.

As used herein, an “AA (without HGDs)” refers to a subject containing an Advanced Adenoma without high-grade dysplasia. For example, an “AA (without HGDs)” refers to a subject containing at least one of (i) a polyp measuring ≥1 cm in the greatest dimension and (iii) a tubulovillous/villous polyp of any size, but does not contain (ii) a polyp of any size with high-grade dysplasia.

As used herein, an “AA (HGDs)” refers to a subject containing an Advanced Adenoma with high-grade dysplasia. For example, an “AA (HGDs)” refers to a subject containing a polyp of any size with high-grade dysplasia.

As used herein, a “High-Risk Advanced Adenoma” refers to (i) a tubular adenomas or serrated lesions (except HP) with low-grade dysplasia measuring ≥1.5 cm, (ii) a conventional adenomas or serrated lesions with high-grade dysplasia of any size, (iii) a tubulovillous/villous polyps of any size, and (iv) a combination thereof. The term HP is an abbreviation for hyperplastic polyps.

As used herein, a “High-Risk Advanced Adenoma (high grade dysplasia only)” refers to a conventional adenomas or serrated lesions with high-grade dysplasia of any size.

As used herein “Low-Risk Advanced Adenoma” refers to a subject containing one of a (i) tubular adenomas, (ii) SSA, and (iii) TSA measuring ≥1 cm-1.4 cm, and (iv) HP of any size. The term SSA is an abbreviation for sessile serrated adenoma and TSA is an abbreviation for traditional serrated adenomas.

As used herein, a “Non-Advanced Adenoma” refers to a subject containing one of inflammatory polyps of any size, polypoid colon mucosa of any size, tubular adenoma <10 mm, sessile serrated adenoma <10 mm, traditional serrated adenoma <10 mm, and hyperplastic polyps <10 mm. Similarly, “Non-Advanced Adenoma” can refer to a subject having an adenoma and that the subject does not have the advanced adenoma disease state

As used herein, “Colonoscopy Negative Control” refers to a subject who had a colonoscopy where no polyp, lesion, or abnormal tissue was found in the colon. In various embodiments, the colonoscopy can be a recent procedure where the blood sample is taken from the subject within 1 day, 1 week, 4 weeks, 10 weeks, or 6 months before or after the colonoscopy procedure.

### III. Overview of Exemplary Workflow

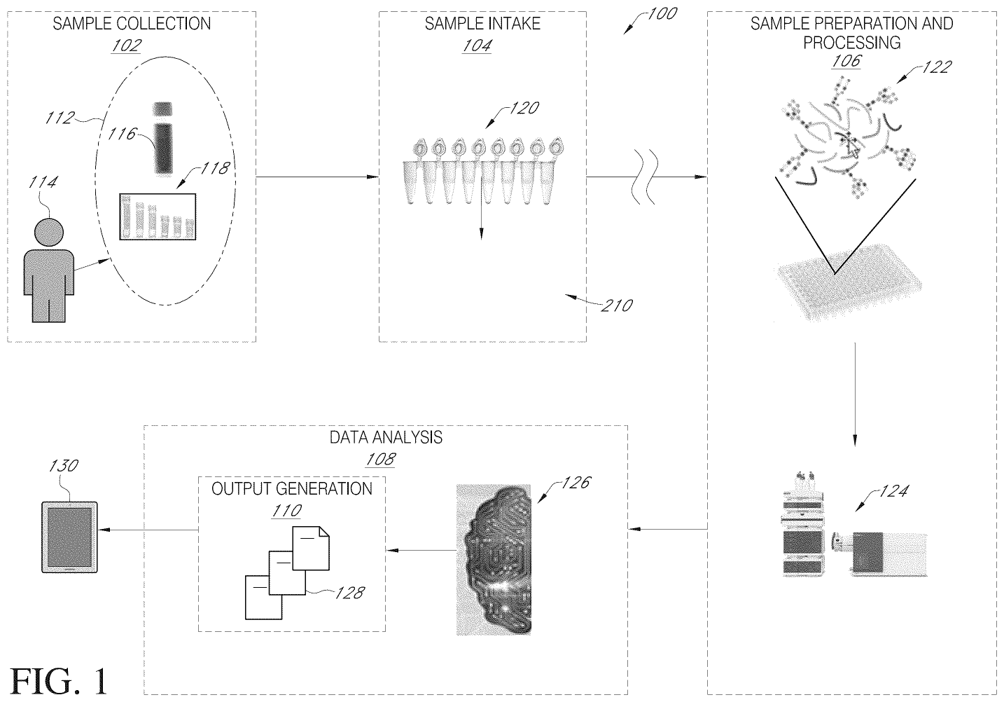

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw into a tube. In some situations, a phlebotomist inserts a hollow needle into an arm of a subject such that the needle pierces a vein. The hollow needle is attached to one end of a flexible conduit and the other end of the flexible conduit can subsequently be coupled to the tube. The tube may be at a lower pressure than the ambient pressure outside of the tube causing a blood sample to flow into the tube. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, the tube can be a Streck tube (La Vista, Nebraska, USA) or a Becton Dickinson (BD) Vacutainer SST tube (serum sample tubes, Franklin Lakes, New Jersey, USA). The Streck tube can be a RNA Complete BCT, Cell-Free DNA BCT, Cyto-Chex BCT, or ESR-Vacuum tube. In various embodiments of a method for collecting blood, the tubes described herein can be used for collecting a blood sample that is used for determining whether a subject has CRC/AA or is likely to develop CRC.

In various embodiments, the tube for collecting blood can include an anticoagulant and a preserving agent. The anticoagulant can prevent the formation of a clot with the biological sample. The anticoagulant may be one of citrate salt, EDTA salt, and a combination thereof. The salt of the anticoagulant can be one of lithium, potassium, and sodium, and combinations thereof. The preserving agent can be one that is configured to release a formaldehyde or other chemical species that includes an aldehyde moiety. The formaldehyde or aldehyde moiety can form a Schiff base with reactive amine groups on proteins or glycoproteins that in turn reduces metabolic activity in the blood sample and/or stabilizes the structural integrity of the cell membrane of the various cells in the blood sample. Under certain circumstances, the formaldehyde or aldehyde moiety may crosslink or partially crosslink a cell membrane and proteins and glycoproteins in the blood sample. An example of a preserving agent configured to release a formaldehyde or other chemical species that includes an aldehyde moiety is imidazolidinyl urea (IDU). For situations where the released amounts of formaldehyde or aldehyde moiety groups need to be limited, the preserving agent can also include a quenching agent such as, for example, glycine. Quenching agents such as glycine have amine groups that can react with any generated formaldehyde or other aldehyde moieties. In an embodiment, a combination that includes IDU and glycine may be referred to as an aldehyde-free preserving agent.

An embodiment of a DNA Complete BCT tube (or other non-Streck tube) can include about 50 μl to about 400 μl of a protective agent in a tube and be used as a container for collecting blood. The protective agent can include imidazolidinyl urea (IDU), ethylenediamine tetraacetic acid (EDTA), and glycine. A blood sample having a first concentration of a protein, a glycoprotein, a peptide, or a glycopeptide can be drawn into a tube, whereby it contacts the protective agent. A plasma fraction can be isolated from the contacted blood sample after the blood draw. The isolating of the plasma sample can be performed after the contacting of the blood with the protective agent for at least about 3 minutes, 5 minutes, 10 minutes, 1 hour, 24 hours, 5 days, 7 days, and 14 days. In another embodiment, a time in between the isolating of the plasma sample and the contacting of the blood with the protective agent ranges from about 3 minutes to 14 days, 30 minutes to 7 days, 12 hours to 7 days, 24 hours to 7 days, and 24 hours to 3 days. The concentration of the imidazolidinyl urea after the contacting step can be about or greater than 5 mg/ml. The concentration of the glycine after the contacting step can be about or below about 0.03 g/ml. The protective agent can be present in an amount that can be about or less than about 5% of an overall mixture volume of the protective agent and the drawn blood sample. In various embodiments, this method of collecting blood can be free of any step of cooling or refrigerating the contacted blood sample to a temperature below room temperature after it has been contacted with the protective agent composition. In various embodiments, this method of collecting blood can be performed at ambient room temperature (e.g., 20 to 25° C.). Optionally, after the isolating of the plasma fraction, the plasma fraction can then be stored at a reduced temperature than ambient (e.g., 15 to 3.3° C.) or frozen (e.g., <0° C.). The isolating of the plasma fraction can be performed by centrifuging the tube to cause the cells to aggregate at the bottom of the tube and leaving the plasma fraction at the top portion of the tube. In an embodiment, as a result of metabolic inhibition of the blood cells in the treated blood sample by one or all of the components of the protective agent, apoptotic and necrotic pathways are inhibited and the blood cells (e.g., red or white blood cells), proteins, glycoproteins, peptides, and/or glycopeptides are protected from degradation. In various embodiments, after at least 24 hours, the contacted blood sample has a second concentration of the protein, the glycoprotein, the peptide, or the glycopeptide where the second concentration is not lower or higher than the first concentration by any statistically significant value. For example, the p value can be >0.05 indicating that there is no statistical difference between the first and second concentrations. In another example, the first and second concentration can have a % difference change of less than a 10%, 20%, 30%, 40%, or 50% (absolute value).

In various embodiments, the tube can contain a concentration of the IDU prior to the contacting step that can be between about 0.1 g/mL and about 3 g/mL. A concentration of the protective agent after the contacting step can be less than about 0.8 g/mL. A concentration of the glycine after the contacting step can be below about 0.03 g/mL

The protective agent stabilizes blood cells in the blood sample to reduce or eliminate the rupture and/or degradation of the blood cells (e.g., white or red) so as to reduce or prevent the release of cellular components. In various embodiments, IDU releases an amount of a formaldehyde releaser preservative agent (e.g., formaldehyde) and the glycine is configured to quench any formaldehyde releaser preservative agent. In combination, IDU and glycine can form an aldehyde-free preservative agent. Under certain circumstances when an assay is designed to only measure circulating glycoproteins, proteins, peptides, and/or glycopeptides outside of the cells for classifying whether a subject has CRC/AA, it can be desirable to substantially reduce or eliminate the rupture and/or degradation of the blood cells. In addition, the rupture of red blood cells can release a relatively large concentration of the hemoglobin, which is a glycoprotein, and can compete or interfere with the measurement of circulating proteins, glycoproteins, peptides and/or glycopeptides. For example, a relatively high hemoglobin concentration can interfere with the efficiency of the proteolytic digestion process especially for the situation where the hemoglobin concentration is much greater than or similar to a concentration of a targeted glycoprotein, glycopeptide, protein, and/or peptide for measurement.

In various embodiments, EDTA will bind divalent ions such as Mg²⁺ and Ca²⁺ that can slow, stop, or prevent a coagulation process inside of a tube used for blood collection. The EDTA can be in the form of an ETDA salt having 1, 2, or 3 sodium or potassium ions such as for example K₃EDTA or K₂EDTA.

In another embodiment of a DNA Complete BCT tube (or other non-Streck tube) can include at least, or about, 200 grams per liter of a composition formulated for stabilizing proteins, glycoproteins, peptides, and/or glycopeptides within a blood sample. The composition can include a) about 50 to about 500 grams per liter of at least one formaldehyde releaser preservative agent; b) ethylenediaminetetraacetic acid (EDTA); and c) one or more solvent. The presence of the at least one formaldehyde releaser preservative agent results in release of at least some formaldehyde and up to, or about, 1% formaldehyde into the composition. The blood collection tube and composition located therein can be sent to a remote location for collection of a blood sample that contains proteins, glycoproteins, peptides, and/or glycopeptides that are stabilized by the composition. In an embodiment, stabilized can refer to a situation where the concentration does not change statistically significantly for a period of time from the contact of the blood with the composition to the time of the test measurement for the proteins, glycoproteins, peptides, and/or glycopeptides.

In various embodiments, the at least one formaldehyde releaser preservative agent may crosslink proteins or glycoproteins in the tube and then cause an interference with a subsequent measurement of targeted proteins or glycoproteins. For this reason, the at least one formaldehyde releaser preservative agent can be configured to release a targeted amount of formaldehyde such as at least 0.001%, 0.01%, 0.01%, 0.2%, 0.5%, 0.75%, or 1% formaldehyde into the composition.

In various embodiments, a method can include providing an evacuated blood collection tube including at least, or about, 200 grams per liter of a composition formulated for stabilizing proteins or glycoproteins within a blood sample. The composition can include about 50 to about 500 grams per liter of at least one formaldehyde releaser preservative agent, wherein the at least one formaldehyde releaser preservative agent includes imidazolidinyl urea (IDU); ethylenediaminetetraacetic acid (EDTA); one or more solvents; and at least some formaldehyde and up to about 1% formaldehyde as a result of the at least one formaldehyde releaser preservative agent. The blood can be drawn into the evacuated blood collection tube including the composition. The inside portion of an evacuated collection tube has a reduced pressure compared to a pressure outside the tube that facilitates a withdrawal of blood from a subject. After filling a portion of the blood collection tube with blood, the blood collection tube can be sent to a remote location for the isolation of the proteins and glycoproteins in a plasma portion from the stabilized blood sample. Once the blood collection tube with blood is received at the remote location, the plasma portion containing proteins and glycoproteins can be isolated from the stabilized blood sample. The isolated proteins and glycoproteins from the plasma portion of the stabilized blood sample can be tested to identify the presence, absence or severity of a CRC/AA disease state by performing one or more of the following: gel electrophoresis, capillary electrophoresis, western blot, mass spectrometry, liquid chromatography, fluorescence detection, ultraviolet spectrometry, immunoassay, or any combination thereof. The collected blood sample is storable for at least, or about 7 days without cell lysis and without glycoprotein or protein degradation of the blood sample due to metabolism after blood collection.

In various embodiments, solvents suitable for use in the tubes described herein include water, saline, dimethylsulfoxide, alcohol, and any mixture thereof.

In various embodiments, a method for identifying a characteristic of a glycoprotein or protein in a whole blood sample from a subject is described that uses a centrifuge. This method can include positioning a composition including whole blood and a protective agent. The protective agent including at least one preservative agent within a centrifuge. In various embodiments the preservative agent includes one of diazolidinyl urea, imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-aza-3,7-dioxabicyclo[3.3.0]octane, 5-hydroxymethyl-1-aza-3,7-dioxabicyclo[3.3.0]octane, 5-hydroxypoly[methyleneoxy]methyl-1-aza-3,7dioxabicyclo[3.3.0]octane, quaternary adamantine, and any combination thereof. The composition can be centrifuged at a speed of at least about 1000 g and below about 4500 g for at least about 5 minutes and less than about 20 minutes to isolate a plasma fraction that includes the proteins and glycoproteins for further analysis. The isolated proteins and glycoproteins obtained from the plasma fraction can be tested to identify whether the subject has a CRC/AA disease state. In another embodiment, the composition can be centrifuged at a speed of about 1600 g for about 15 minutes to isolate a plasma fraction that includes the proteins and glycoproteins for further analysis.

An embodiment of a Cyto-Chex BCT tube (or other non-Streck tube) can include preloaded compounds consisting of or including ethylene diamine tetra acetic acid (EDTA) and diazolidinyl urea. The tube has an open end and a closed end that receives cells collected directly from a blood draw and wherein a majority of an interior portion of the tube is substantially free of contact with the preloaded components. A blood sample containing a plurality of blood cells can be drawn into the tube whereby it contacts the preloaded compounds to yield a final composition. A ratio of a volume of the preloaded compounds to a combined volume of the blood sample and the preloaded compounds can be from about 1:100 to about 2:100. The plurality of blood cells of the blood sample can be stabilized directly and immediately upon the blood draw. The blood sample can be transported, wherein the blood sample is drawn and transported in the same tube with no processing steps between the blood draw and transporting.

In another embodiment of a Cyto-Chex BCT tube (or other non-Streck tube), it can include a closed collection container having an internal pressure less than atmospheric pressure outside the container. The collection container contains preloaded compounds consisting of or including (i) ethylene diamine tetra acetic acid (EDTA); and (ii) diazolidinyl urea. A majority of an interior portion of the collection container is substantially free of contact with the preloaded component. A blood sample containing the blood cells can be drawn into the collection container whereby the blood sample contacts the preloaded compounds to yield a final composition. After collection of the blood cells in the container, a ratio of a volume of the preloaded compounds to a volume of the final composition can be from about 1:100 to about 2:100.

In yet another embodiment of a Cyto-Chex BCT tube (or other non-Streck tube), it can include a collection container for receiving a whole blood sample. Preloaded compounds can be introduced into the collection container. The preloaded compounds consist of or include (i) ethylene diamine tetra acetic acid (EDTA); and (ii) diazolidinyl urea. The collection container can be evacuated to an internal pressure that is less than atmospheric pressure outside the collection container. A volume of the whole blood sample can be drawn into the collection container, wherein a majority of an interior portion of the collection container is substantially free of contact with the preloaded compounds. The whole blood sample can contact the preloaded compounds to yield a final composition. A ratio of a volume of the preloaded compounds to a volume of the final composition can be from about 1:100 to about 2:100.

In one of the embodiments of the Cyto-Chex BCT tube (or other non-Streck tube), the ratio of the volume of the preloaded compounds to a combined volume of the blood sample and the preloaded compounds can be from about 1:1000 to about 1:10, about 5:1000 to about 5:100, about 1:100 to about 5:100, about 1:100 to about 5:100, and about 1:100 to about 2:100.

An embodiment of a BD Vacutainer® SST tube (or other non-BD tube) can include spray-coated silica and a polymer gel (e.g., polyester based) for serum separation. This type of tube can be used for isolating a serum sample. The spray-coated silica includes silica particles coating an inner surface of the tube. The silica particles are configured to initiate a clot activation in a blood samples. A blood sample itself typically has various components that can create a clot, but requires an activation trigger to start the clotting cascade. However, under certain circumstances, a triggering event can be caused by the contact of the blood with the silica particles coated on an inner wall of the tube. The tube may be inverted at least 5 times and the clotting process can occur, which can take about 30 minutes. After the clotting process has occurred, the tube can be centrifuged to create a serum fraction at a top portion of the tube separate from the blood cells at the bottom of the tube. The centrifugation process may be performed for about 10 minutes at about 1000-1300 RCG (g). The polymer gel forms a physical barrier between the serum fraction and the blood cells during centrifugation that can facilitate the aspiration of the serum fraction.

It is worthwhile to note that although the above description describes the use of a Streck tube, a tube, other than one from Streck, can be used containing one or more of the reagents as described above. Similarly, although the above description describes the use of a BD SST tube, a tube, other than one from BD, can be used containing one or more of the reagents as described above.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### IV. Detection and Quantification of Peptide Structures

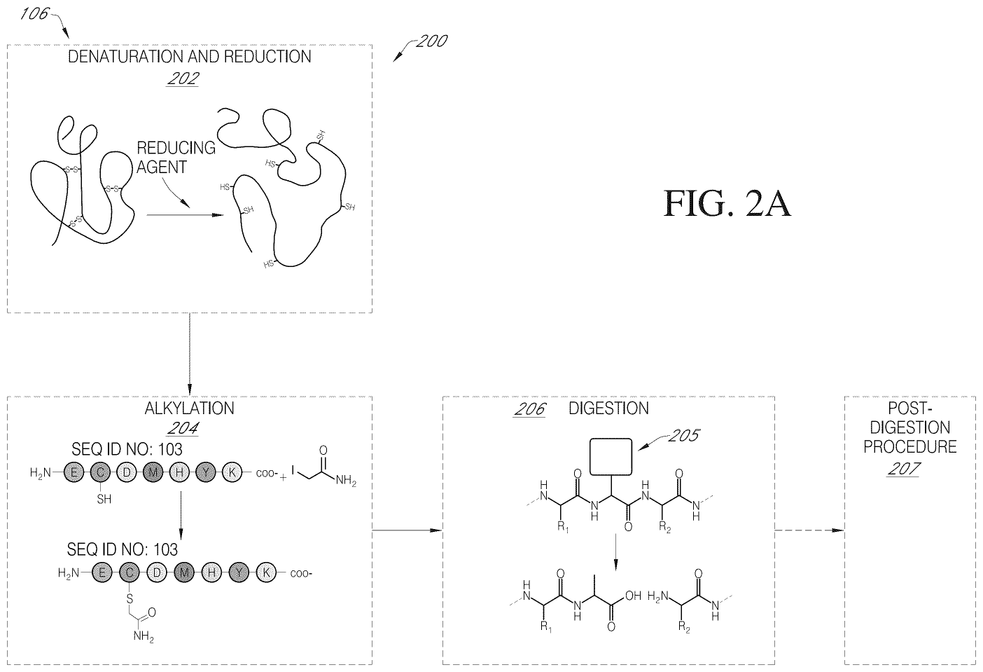

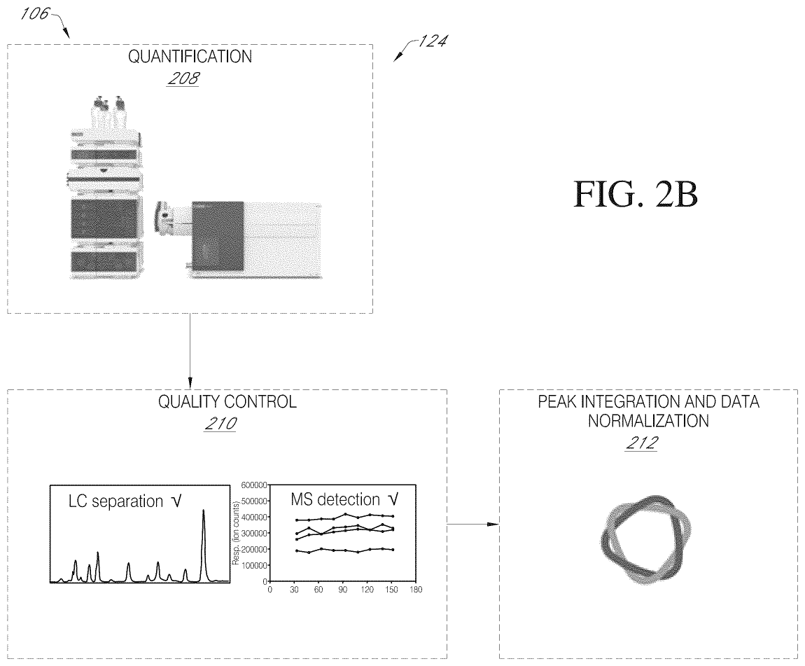

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl) phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant production. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

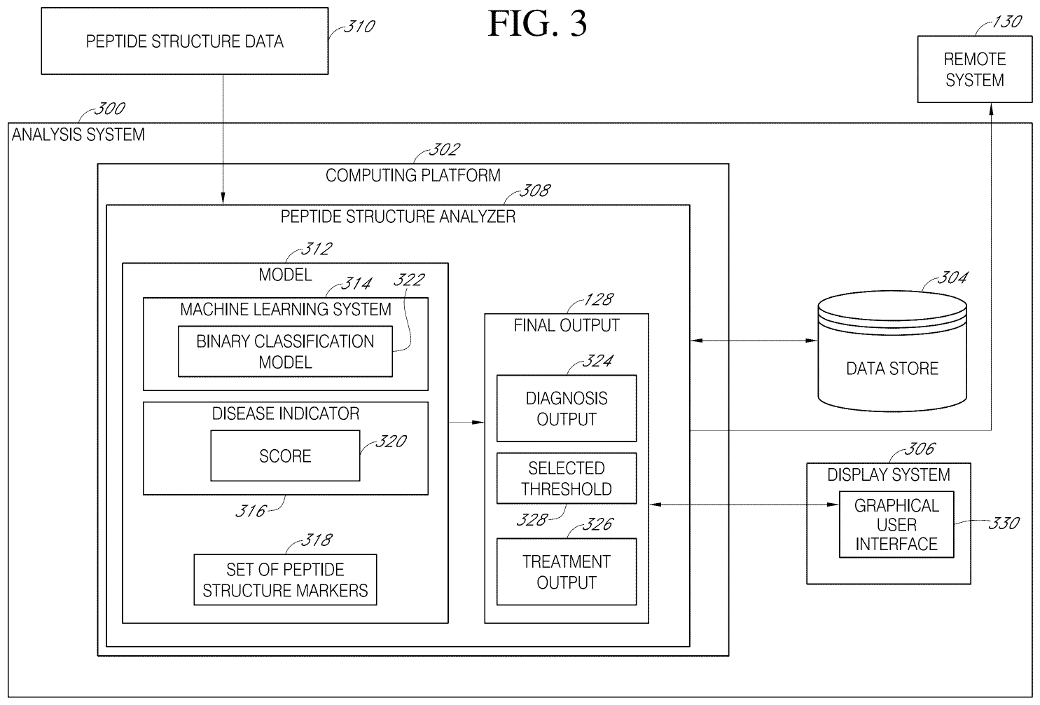

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for a colorectal cancer disease state based on set of peptide structures 318 identified as being associated with the colorectal cancer disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the colorectal cancer disease state. In various embodiments, disease indicator 316 can include a score 320. Score 320 indicates whether the colorectal cancer disease state is present or not. For example, score 320 may be, a probability score that indicates how likely it is that the biological sample 112 evidences the presence of the colorectal cancer disease state.

In one or more embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the colorectal cancer disease state based on training of model 312. In one or more embodiments, set of peptide structures 318 includes at least one, at least two, or at least three peptide structures from a first group of peptide structures (peptide structures PS-1 through PS-31) identified in Table 1. For example, in one or more embodiments, set of peptide structures 318 includes at least 1, at least 2, at least 3, at least 4, at least 5, or all 6 of the peptide structures identified in Table 1. In some cases, the number of peptide structures selected from Table 1 for inclusion in set of peptide structures 318 may be based on, for example, a desired level of accuracy.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In one or more embodiments, final output 128 includes diagnosis output 324, treatment output 326, or both. Diagnosis output 324 may include, for example, a diagnosis for the colorectal cancer disease state. The diagnosis can include a positive diagnosis or a negative diagnosis for the advanced adenoma or colorectal cancer disease state.

In one or more embodiments, when disease indicator 316 and/or diagnosis output 324 indicate a positive diagnosis for the advanced adenoma colorectal cancer disease state, a colonoscopy and/or biopsy may be recommended. For example, a colonoscopy and/or biopsy of the subject may be performed in response to disease indicator 316 and/or diagnosis output 324 indicating a positive diagnosis for the advanced adenoma or colorectal cancer disease state. In some embodiments, peptide structure analyzer 308 (or another system implemented on computing platform 302) may generate a report recommending that a colonoscopy and/or biopsy is to be performed for the subject in response to disease indicator 316 and/or diagnosis output 324 indicating a positive diagnosis for the advanced adenoma or colorectal cancer disease state. In other embodiments, peptide structure analyzer 308 may send diagnosis final output 128 to remote system 130 over one or more wireless, wired, and/or optical communications links and remote system 130 may generate a report recommending that a colonoscopy and/or biopsy is to be performed for the subject in response to disease indicator 316 and/or diagnosis output 324 indicating a positive diagnosis for the advanced adenoma or colorectal cancer disease state. The biopsy may be used to confirm the diagnosis to determine whether or not to administer treatment and/or how quickly to administer treatment. When disease indicator 316 and/or diagnosis output 324 indicate a negative diagnosis for the colorectal cancer disease state (e.g., advanced colon adenoma), the report that is generated by peptide structure analyzer 308, remote system 130, or some other system implemented on computing platform 142 may recommend a period of monitoring for the subject. For example, a negative diagnosis indication by disease indicator 316 and/or diagnosis output 324 may thus help prevent unnecessary treatment or overtreatment of the subject.

Treatment output 326 may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 330 in display system 306 for viewing by a human operator.

### V.A.2. Computer Implemented System

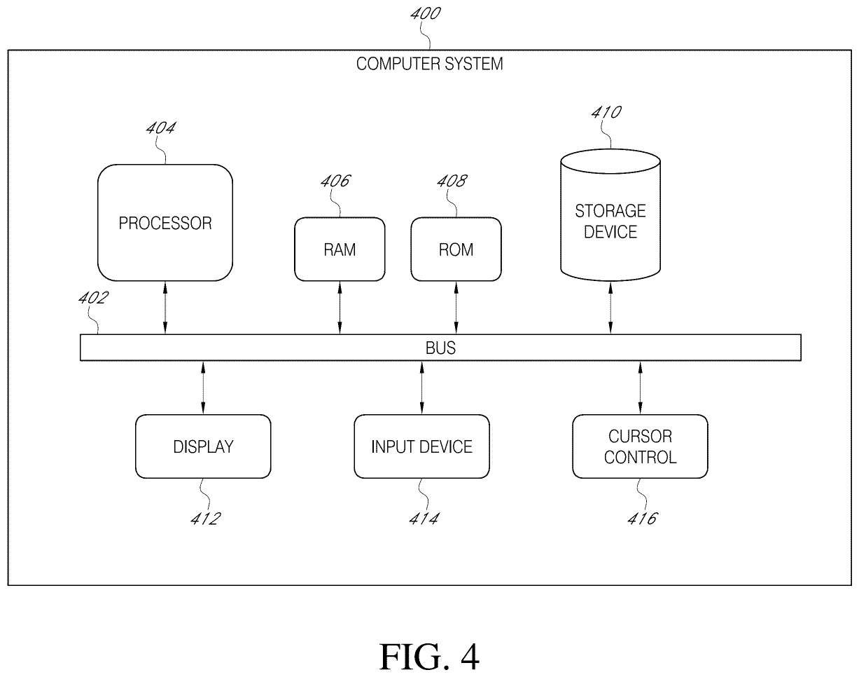

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein.

Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis Based on Peptide Structure Data Analysis

### VI.A.1 Exemplary Methodology-Based on Table 1

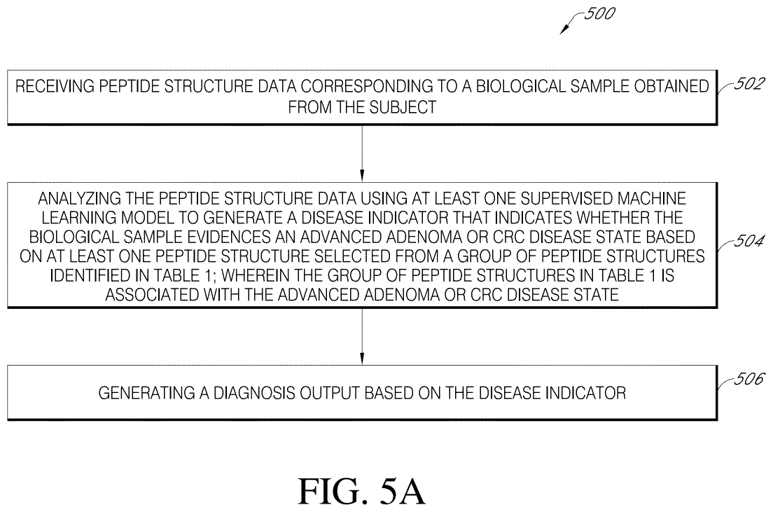

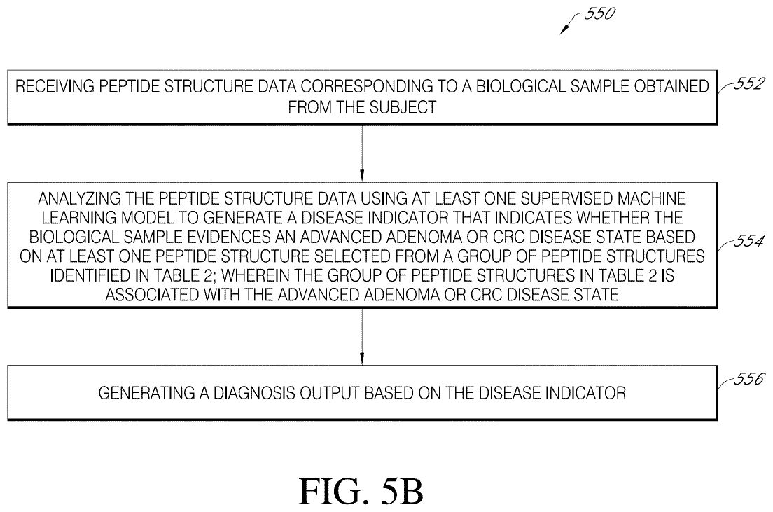

FIG. 5A is a flowchart of a process for diagnosing a subject with respect to advanced adenoma or colorectal cancer (CRC) disease state, in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1, with the peptide sequence being one of SEQ ID NOS: 1-31 in Table 1 below.

Step 504 includes analyzing the peptide structure data using at least one supervised machine learning model to generate a disease indicator that indicates whether the biological sample evidences an advanced adenoma or CRC disease state based on at least one peptide structure selected from a group of peptide structures identified in Table 1. In step 504, in accordance with various embodiments, the group of peptide structures can be associated with the colorectal cancer disease state. In step 504, in accordance with various embodiments, the group of peptide structures can be associated with the advanced adenoma or CRC disease state. In step 504, in accordance with various embodiments, the group of peptide structures can be listed in Table 1 with respect to relative significance to the disease indicator.

The group of peptide structures in Table 1 includes peptide structures that have been determined relevant to distinguishing at least between colorectal cancer (and/or advanced adenoma) and a healthy state. For example, the group of peptide structures may be used to predict the probability of colorectal cancer (and/or advanced adenoma) for use in clinically screening patients. In one or more embodiments, the first group of peptide structures in Table 1 may also be peptide structures that have been determined relevant to distinguishing between colorectal cancer (and/or advanced adenoma) and a healthy state.

In one or more embodiments, the at least 1 peptide structures includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or all 31 of the peptide structures PS-1 to PS-31 in Table 1.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

In some embodiments, step 504 may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the advanced adenoma or colorectal cancer disease state and the supervised machine learning model is configured to generate an output that identifies the biological sample as either evidencing (“positive for”) the advanced adenoma or colorectal cancer disease state when the disease indicator is greater than a selected threshold or not evidencing (“negative for”) the advanced adenoma colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the advanced adenoma or colorectal cancer disease state if the biological sample evidences the advanced adenoma or colorectal cancer disease state based on the disease indicator. The diagnosis may be, for example, “negative” if the biological sample does not evidence the advanced adenoma or colorectal cancer disease state based on the disease indicator. A negative diagnosis may mean that the biological sample has a non-colorectal cancer state. The negative diagnosis for the advanced adenoma or colorectal cancer disease state can include at least one of a healthy state, or some other non-malignant state.

Generating the diagnosis output in step 506 may include determining that the score falls above (or at or above) a selected threshold and generating a positive diagnosis for the colorectal cancer disease state. Alternatively, step 506 can include determining that the score falls below (or at or below) a selected threshold and generating a negative diagnosis for the advanced adenoma or colorectal cancer disease state. In some scoring systems, the score can include a probability score and the selected threshold can be 0.5. In other scoring systems, the selected threshold can fall within a range between 0.30 and 0.65.

In one or more embodiments, the final output in step 506 may include a treatment output if the diagnosis output indicates a positive diagnosis for the colorectal cancer disease state or advanced adenoma disease state. The treatment output may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 1 below lists a first group of peptide structures associated with malignant colorectal cancer (and/or advanced adenoma disease state). One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 1
31 Peptide Structures Associated with AA and/or Colorectal Cancer (CRC)

| | | | Prot | Pep | Glycos site | Glyco site | Glycan | Mono- |
|---|---|---|---|---|---|---|---|---|
| PS-ID | | | SEQ | SEQ | within | within | Struct GL | isotopic |
| No. | PS-NAME | Protein Name | ID NO | ID NO | Prot SEQ | Pept SEQ | No. | mass |
| 1 | A1AT_271_5401 | Alpha-1- | 36 | 1 | 271 | 4 | 5401 | 3668.565 |
| | | antitrypsin | | | | | | |
| 2 | A1AT_271_5412 | Alpha-1- | 36 | 2 | 271 | 4 | 5412 | 4105.718 |
| | | antitrypsin | | | | | | |
| 3 | AFAM_33_5402 | Afamin | 37 | 3 | 33 | 6 | 5402 | 3399.324 |
| 4 | AGP1_93_7613 | Alpha-1-acid | 38 | 4 | 93 | 7 | 7613 | 5287.079 |
| | | glycoprotein 1 | | | | | | |
| 5 | AGP12_56_6503 | Alpha-1-acid | 38 or 39 | 5 | 56 | 5 | 6503 | 3656.34 |
| | | glycoprotein 1or2 | | | | | | |
| 6 | ANGT_47_5401 | Angiotensinogen | 40 | 6 | 47 | 12 | 5401 | 4447.951 |
| 7 | ANT_128_5402 | Antithrombin-III | 41 | 7 | 128 | 5 | 5402 | 4070.674 |
| 8 | ANT_187_5412 | Antithrombin-III | 41 | 8 | 187 | 5 | 5412 | 4527.883 |
| 9 | APOE_212_NONGLYCOSYLATED | Apolipoprotein E | 42 | 9 | N/A | N/A | N/A | 1496.795 |
| 10 | APOH_253_NONGLYCOSYLATED | Beta-2- | 43 | 10 | N/A | N/A | N/A | 1249.558 |
| | | glycoprotein1 | | | | | | |
| 11 | CERU_358_5402 | Ceruloplasmin | 44 | 11 | 358 | 13 | 5402 | 3843.555 |
| 12 | CERU_358_NONGLYCOSYLATED | Ceruloplasmin | 44 | 12 | N/A | N/A | N/A | 1638.782 |
| 13 | CFAI_70_5401 | Complement | 45 | 13 | 70 | 1 | 5401 | 2976.165 |
| | | Factor I | | | | | | |
| 14 | CO3_85_6200 | ComplementC3 | 46 | 14 | 85 | 12 | 6200 | 3632.628 |
| 15 | FETUA_156_6513 | Alpha-2-HS- | 47 | 15 | 156 | 12 | 6513 | 4777.897 |
| | | glycoprotein | | | | | | |
| 16 | FINC_542_6502 | Fibronectin | 48 | 16 | 542 | 8 | 6502 | 4501.687 |
| 17 | HEMO_453_5402 | Hemopexin | 49 | 17 | 453 | 7 | 5402 | 3939.645 |
| 18 | HEMO_64_5402 | Hemopexin | 49 | 18 | 64 | 15 | 5402 | 4731.84 |
| 19 | HPT_241_5402 | Haptoglobin | 50 | 19 | 241 | 6 | 5402 | 3998.776 |
| 20 | HPT_241_6512 | Haptoglobin | 50 | 20 | 241 | 6 | 6512 | 4509.966 |
| 21 | HPT_184_7602 | Haptoglobin | 50 | 21 | 184 | 6 | 7602 | 5613.422 |
| 22 | IC1_238_5402 | Plasma protease | 51 | 22 | 238 | 5 | 5402 | 3113.208 |
| | | C1 inhibitor | | | | | | |
| 23 | IC1_238_5411 | Plasma protease | 51 | 23 | 238 | 6 | 5411 | 2968.17 |
| | | C1 inhibitor | | | | | | |
| 24 | IC1_253_5412 | Plasma protease | 51 | 24 | 253 | 4 | 5412 | 4450.915 |
| | | C1 inhibitor | | | | | | |
| 25 | IGG1_297_3310 | Immunoglobulin | 52 | 25 | 180 | 5 | 3310 | 2429.959 |
| | | heavy constant | | | | | | |
| | | gamma 1 | | | | | | |
| 26 | IGG1_297_3410 | Immunoglobulin | 52 | 26 | 180 | 5 | 3410 | 2633.039 |
| | | heavy constant | | | | | | |
| | | gamma 1 | | | | | | |
| 27 | KLKB1_396_5401 | Plasma Kallikrein | 53 | 27 | 396 | 6 | 5401 | 4270.857 |
| 28 | THRB_416_5402 | Prothrombin | 54 | 28 | 416 | 1 | 5402 | 3424.392 |
| 29 | TRFE_630_5402 | Serotransferrin | 55 | 29 | 630 | 9 | 5402 | 4718.889 |
| 30 | TRFE_630_5402_NH3loss | Serotransferrin | 55 | 30 | 630 | 9 | 5402 | 4701.863 |
| 31 | VTNC_169_5402 | Vitronectin | 56 | 31 | 169 | 1 | 5402 | 3115.238 |

### VI.A.2 Exemplary Methodology-Based on Table 2

In another embodiment, a method for diagnosing a subject that has a likelihood of having advanced adenomas (AA) or a colorectal cancer (CRC) disease state can be implemented using one or more of the biomarkers listed in Table 2 (see FIG. 5B). Once it is established that there is a likelihood of having advanced adenomas or colorectal cancer (CRC) disease state, a recommendation to perform a colonoscopy can be provided to a subject. If it is not established that there is a likelihood having advanced adenomas or colorectal cancer (CRC) disease state, a recommendation to not perform a colonoscopy can be provided to a subject. By using a screening test based on a blood sample that assesses the likelihood of having a condition and can potentially recommend no need to perform a colonoscopy, the subject can avoid an unnecessary colonoscopy that is unpleasant and expensive. Under certain conditions, the term likelihood may be referred to as a probability. It is worthwhile to note that a test using samples such as serum or plasma (blood based) are much more convenient than a colonoscopy procedure that will likely improve compliance in monitoring for CRC/AA.

A process 550 for diagnosing a subject that has a likelihood of having AA or a colorectal cancer (CRC) disease state can include a step 552 to receive peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 2, with the peptide sequence being one of SEQ ID NOS: 3-9, 12, 14-16, 18, 25-28, and 31-35 in Table 2 below.

The method for diagnosing a subject that has a likelihood of having AA or a colorectal cancer (CRC) disease state can also include a step 554 to analyze the peptide structure data using at least one supervised machine learning model to generate a disease indicator that indicates whether the biological sample evidences a likelihood of an AA or CRC disease state based on at least one peptide structure selected from a group of peptide structures identified in Table 2. In accordance with various embodiments, the group of peptide structures can be associated with the colorectal cancer disease state. The group of peptide structures can be associated with the advanced adenoma or CRC disease state.

The group of peptide structures in Table 2 includes peptide structures that have been determined relevant to distinguishing at least between colorectal cancer/AA and a healthy state. For example, the group of peptide structures may be used to predict the probability of colorectal cancer/AA for use in clinically screening patients. In one or more embodiments, the first group of peptide structures in Table 2 may also be peptide structures that have been determined relevant to distinguishing between colorectal cancer/AA and a healthy state.

In one or more embodiments, the at least 1 peptide structures include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 21 of the peptide structures PS-ID NO: 3-9, 12, 14-16, 18, 25-28, and 31-35 in Table 2.

The method for diagnosing a subject that has a likelihood of having AA or a colorectal cancer (CRC) disease state may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

The method for diagnosing a subject that has a likelihood of having AA or a colorectal cancer (CRC) disease state may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for either AA or colorectal cancer disease state and the supervised machine learning model is configured to generate an output that identifies the biological sample as either evidencing (“positive for”) the AA or colorectal cancer disease state when the disease indicator is greater than a selected threshold or not evidencing (“negative for”) the advanced adenoma or colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

The method for diagnosing a subject that has a likelihood of having AA or a colorectal cancer (CRC) disease state can include a step 556 to generate a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the AA or colorectal cancer disease state if the biological sample evidences the AA or colorectal cancer disease state based on the disease indicator. The diagnosis may be, for example, “negative” if the biological sample does not evidence the AA or colorectal cancer disease state based on the disease indicator. A negative diagnosis may mean that the biological sample has a non-colorectal cancer state. The negative diagnosis for the AA or colorectal cancer disease state can include at least one of a healthy state, non-AA, or some other non-malignant state.

Generating the diagnosis output may include determining that the score falls above (or at or above) a selected threshold and generating a positive diagnosis for the colorectal cancer disease/AA state. Alternatively, the diagnosis output can include determining that the score falls below (or at or below) a selected threshold and generating a negative diagnosis for the AA/colorectal cancer disease state. In some scoring systems, the score can include a probability score and the selected threshold can be 0.5. In other scoring systems, the selected threshold can fall within a range between 0.30 and 0.65.

In one or more embodiments, the final output of the method may include a treatment output if the diagnosis output indicates a positive diagnosis for the AA/colorectal cancer disease state. The treatment output may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 2 below lists a first group of peptide structures associated with malignant colorectal cancer and AA. One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 2
Alternative Set of 21 Peptide Structures Associated with CRC/AA.

| | | | Prot | Pep | Glycos site | Glyco site | Glycan | Mono- |
|---|---|---|---|---|---|---|---|---|
| PS-ID | | | SEQ ID | SEQ ID | within | within | Struct | isotopic |
| No. | PS-NAME | Protein Name | NO | NO | Prot SEQ | Pept SEQ | GL No. | mass |
| 32 | AACT_106_7624 | Alpha-1- | 57 | 32 | 106 | 2 | 7624 | 6208.530316 |
| | | antichymotrypsin | | | | | | |
| 3 | AFAM_33_5402 | Afamin | 37 | 3 | 33 | 6 | 5402 | 3399.324044 |
| 4 | AGP1_93_7613 | Alpha-1-acid | 38 | 4 | 93 | 7 | 7613 | 5287.079448 |
| | | glycoprotein 1 | | | | | | |
| 5 | AGP12_56_6503 | Alpha-1-acid | 38 or 39 | 5 | 56 | 5 | 6503 | 3656.339868 |
| | | glycoprotein 1 or 2 | | | | | | |
| 6 | ANGT_47_5401 | Angiotensinogen | 40 | 6 | 47 | 12 | 5401 | 4447.950938 |
| 7 | ANT_128_5402 | Antithrombin-III | 41 | 7 | 128 | 5 | 5402 | 4070.673904 |
| 8 | ANT_187_5412 | Antithrombin-III | 41 | 8 | 187 | 5 | 5412 | 4527.88308 |
| 9 | APOE_212_NONGLYCOSYLATED | Apolipoprotein E | 42 | 9 | N/A | N/A | N/A | 1496.794668 |
| 12 | CERU_358_NONGLYCOSYLATED | Ceruloplasmin | 44 | 12 | N/A | N/A | N/A | 1638.782388 |
| 33 | CERU_397_5402 | Ceruloplasmin | 44 | 33 | 397 | 2 | 5402 | 4330.763968 |
| 14 | CO3_85_6200 | Complement C3 | 46 | 14 | 85 | 12 | 6200 | 3632.628438 |
| 15 | FETUA_156_6513 | Alpha-2-HS- | 47 | 15 | 156 | 12 | 6513 | 4777.897142 |
| | | glycoprotein | | | | | | |
| 16 | FINC_542_6502 | Fibronectin | 48 | 16 | 542 | 8 | 6502 | 4501.68705 |
| 18 | HEMO_64_5402 | Hemopexin | 49 | 18 | 64 | 15 | 5402 | 4731.839512 |
| 34 | HPT_241_6513 | Haptoglobin | 50 | 34 | 241 | 6 | 6513 | 4801.061826 |
| 25 | IGG1_297_3310 | Immunoglobulin | 52 | 25 | 180 | 5 | 3310 | 2429.959172 |
| | | heavy constant | | | | | | |
| | | gamma 1 | | | | | | |
| 26 | IGG1_297_3410 | Immunoglobulin | 52 | 26 | 180 | 5 | 3410 | 2633.03854 |
| | | heavy constant | | | | | | |
| | | gamma 1 | | | | | | |
| 35 | IGG1_297_3510 | Immunoglobulin | 52 | 35 | 180 | 5 | 3510 | 2836.117908 |
| | | heavy constant | | | | | | |
| | | gamma 1 | | | | | | |
| 27 | KLKB1_396_5401 | Plasma | 53 | 27 | 396 | 6 | 5401 | 4270.85736 |
| | | Kallikrein | | | | | | |
| 28 | THRB_416_5402 | Prothrombin | 54 | 28 | 416 | 1 | 5402 | 3424.392064 |
| 31 | VTNC_169_5402 | Vitronectin | 56 | 31 | 169 | 1 | 5402 | 3115.238472 |

VI.B.1 Training the Model to Diagnose with Respect to the CRC Disease State—Based on Table 1

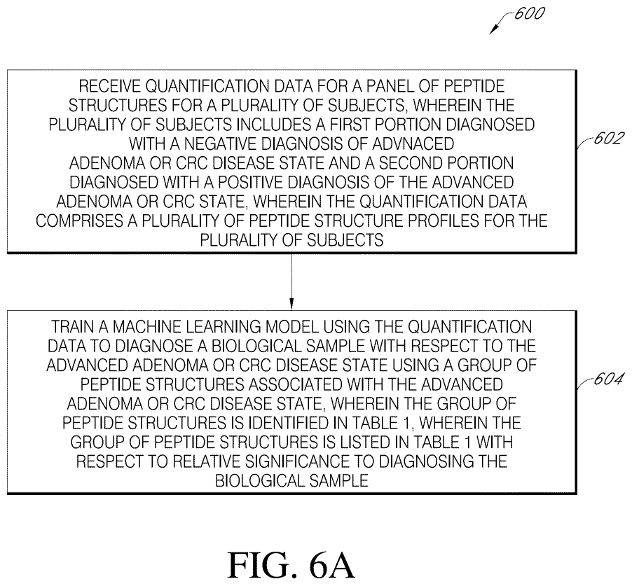

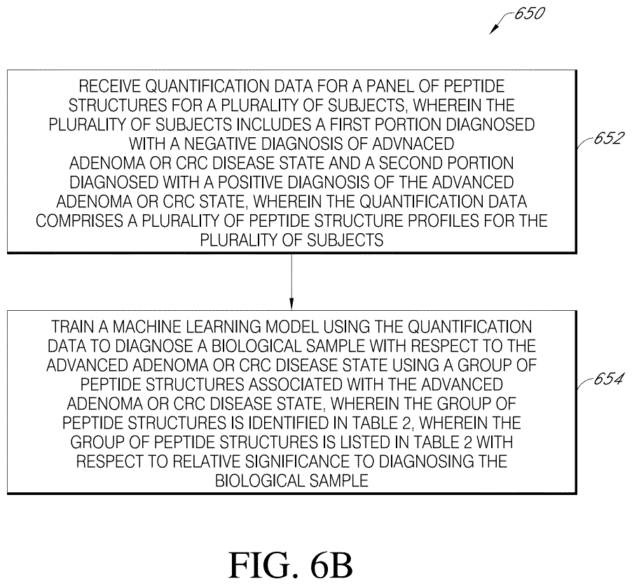

FIG. 6A is a flowchart of a process for training a model to diagnose a subject with respect to an advanced adenoma or CRC disease state in accordance with one or more embodiments. Process 600 may be implemented using, for example, at least a portion of workflow 100 as described in FIGS. 1, 2, and/or analysis system 300 as described in FIG. 3. In some embodiments, process 600 may be one example of an implementation for training the model used in process 500 of FIG. 5A.

Step 602 includes receiving quantification data for a panel of peptide structures for a plurality of subjects. The plurality of subjects includes a first portion diagnosed with a negative diagnosis of an advanced adenoma or CRC disease state and a second portion diagnosed with a positive diagnosis of the advanced adenoma or CRC disease state. The quantification data comprises a plurality of peptide structure profiles for the plurality of subjects.

Step 604 includes training a machine learning model using the quantification data to diagnose a biological sample with respect to the advanced adenoma or CRC disease state using a group of peptide structures associated with the advanced adenoma or CRC disease state (e.g., the group of peptide structures is identified in Table 1). The group of peptide structures is listed in Table 1 with respect to relative significance to diagnosing the biological sample. Step 604 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the advanced adenoma or CRC disease state and a negative diagnosis for any subject of the plurality of subjects determined not to have the advanced adenoma or CRC disease state.

The machine learning model can include a binary classification model. Some binary classification models can include logistical regression models. Some logistical regression models can include LASSO regression models.

An alternative or additional step in process 600 can include performing a differential expression analysis using initial training data to compare a first portion of the plurality of subjects diagnosed with the positive diagnosis for the advanced adenoma or CRC disease state versus a second portion of the plurality of subjects diagnosed with the negative diagnosis for the advanced adenoma or CRC disease state.

An alternative or additional step in process 600 can include identifying a training group of peptide structures based on the differential expression analysis for use as prognostic markers for the advanced adenoma or CRC disease state.

An alternative or additional step in process 600 can include forming the training data based on the training group of peptide structures identified.

An alternative or additional step in process 600 can include identifying a training group of peptide structures based on the differential expression analysis, wherein the training group of peptide structures is a subset of the plurality of peptide structures relevant to diagnosing the advanced adenoma or CRC disease state. The subset may be identified based on at least one of fold-changes, false discovery rates, or p-values computed as part of the differential expression analysis.

An alternative or additional step in process 600 can include training a machine learning model, using the quantification data for the training group of peptide structures, to diagnose a subject of a biological sample with respect to the advanced adenoma or CRC disease state using a group of peptide structures associated with the advanced adenoma or CRC disease state. The group of peptide structures may be a subset of the training group of peptide structures and is identified in Table 1. The group of peptide structures is listed in Table 1 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 1. The markers used for training of the LASSO regression model may, in one or more embodiments, additionally include one or more other peptide structure markers.

VI.B.2 Training the Model to Diagnose with Respect to the CRC Disease State—Based on Table 2

FIG. 6B is a flowchart of a process for training a model to diagnose a subject with respect to an advanced adenoma or CRC disease state in accordance with one or more embodiments. Process 650 may be implemented using, for example, at least a portion of workflow 100 as described in FIGS. 1, 2, and/or analysis system 300 as described in FIG. 3. In some embodiments, process 650 may be one example of an implementation for training the model used in the process 550 of FIG. 5B.

Step 652 includes receiving quantification data for a panel of peptide structures for a plurality of subjects. The plurality of subjects includes a first portion diagnosed with a negative diagnosis of an advanced adenoma or CRC disease state and a second portion diagnosed with a positive diagnosis of the advanced adenoma or CRC disease state. The quantification data comprises a plurality of peptide structure profiles for the plurality of subjects.

Step 654 includes training a machine learning model using the quantification data to diagnose a biological sample with respect to the advanced adenoma or CRC disease state using a group of peptide structures associated with the advanced adenoma or CRC disease state (e.g., the group of peptide structures is identified in Table 2). The group of peptide structures is listed in Table 2 with respect to relative significance to diagnosing the biological sample. Step 654 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the advanced adenoma or CRC disease state and a negative diagnosis for any subject of the plurality of subjects determined not to have the advanced adenoma or CRC disease state.

The machine learning model can include a binary classification model. Some binary classification models can include logistical regression models. Some logistical regression models can include LASSO regression models.

An alternative or additional step in process 650 can include performing a differential expression analysis using initial training data to compare a first portion of the plurality of subjects diagnosed with the positive diagnosis for the advanced adenoma or CRC disease state versus a second portion of the plurality of subjects diagnosed with the negative diagnosis for the advanced adenoma or CRC disease state.

An alternative or additional step in process 650 can include identifying a training group of peptide structures based on the differential expression analysis for use as prognostic markers for the advanced adenoma or CRC disease state.

An alternative or additional step in process 650 can include forming the training data based on the training group of peptide structures identified.

An alternative or additional step in process 650 can include identifying a training group of peptide structures based on the differential expression analysis, wherein the training group of peptide structures is a subset of the plurality of peptide structures relevant to diagnosing the advanced adenoma or CRC disease state. The subset may be identified based on at least one of fold-changes, false discovery rates, or p-values computed as part of the differential expression analysis.

An alternative or additional step in process 650 can include training a machine learning model, using the quantification data for the training group of peptide structures, to diagnose a subject of a biological sample with respect to the advanced adenoma or CRC disease state using a group of peptide structures associated with the advanced adenoma or CRC disease state. The group of peptide structures may be a subset of the training group of peptide structures and is identified in Table 2. The group of peptide structures is listed in Table 2 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 2. The markers used for training of the LASSO regression model may, in one or more embodiments, additionally include one or more other peptide structure markers.

### VI.C.1 Monitoring a Subject for an Advanced Adenoma or Colorectal Cancer Disease State—Based on Table 1

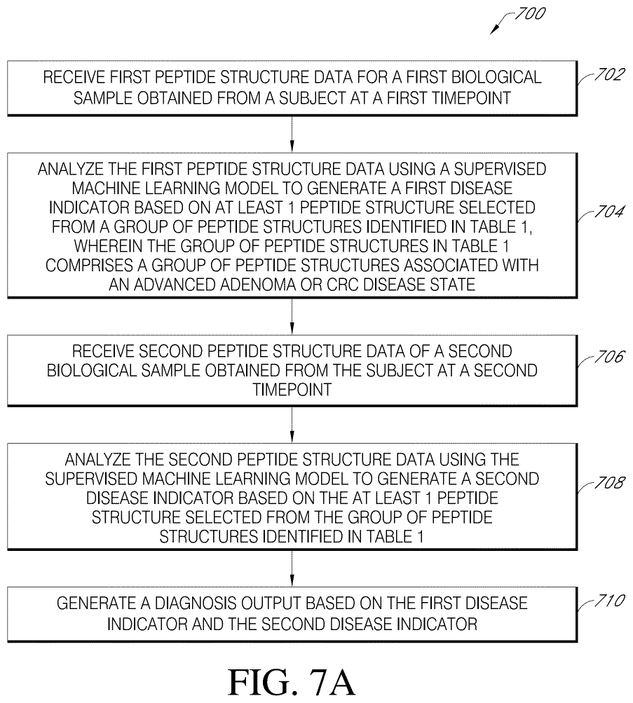

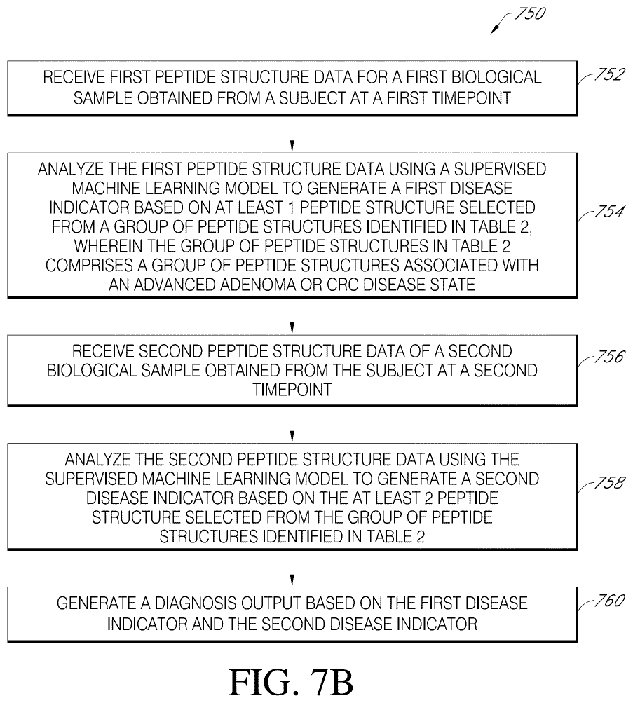

FIG. 7A is a flowchart of a process for monitoring a subject for an advanced adenoma or Colorectal Cancer (CRC) disease state in accordance with one or more embodiments. Process 700 may be implemented using, for example, at least a portion of workflow 100 as described in FIGS. 1, 2, and/or analysis system 300 as described in FIG. 3.

Step 702 includes receiving first peptide structure data for a first biological sample obtained from a subject at a first timepoint.

Step 704 includes analyzing the first peptide structure data using a supervised machine learning model to generate a first disease indicator based on at least 1 peptide structure selected from a group of peptide structures identified in Table 1. The group of peptide structures in Table 1 includes a group of peptide structures associated with an advanced adenoma or CRC disease state in accordance with various embodiments. The supervised machine can be a binary classification model. In some embodiments, the binary classification model can be a logistical regression model.

Step 706 includes receiving second peptide structure data of a second biological sample obtained from the subject at a second timepoint.

Step 708 includes analyzing the second peptide structure data using the supervised machine learning model to generate a second disease indicator based on the at least 1 peptide structure selected from the group of peptide structures identified in Table 1.

Step 710 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the advanced adenoma or CRC disease state and the second biological sample evidences the positive diagnosis for the advanced adenoma or CRC disease. In other embodiments, the diagnosis output identifies whether a non-adenoma or non-CRC disease state has progressed to the advanced adenoma or CRC disease state, respectively, wherein the non-adenoma or non-CRC disease state includes either a healthy state, or a control state.

In accordance with various embodiments, a method is provided for identifying and managing a subject at risk of an advanced adenoma or CRC disease state. The method can comprise receiving a biological sample from the subject, determining a quantity of at least 1 peptide structure identified in Table 1 in the biological sample, analyzing the quantity of each peptide structure using at least one machine learning model to generate a disease indicator, generating a diagnosis output based on the disease indicator that classifies the biological sample as evidencing that the subject has a risk for advanced adenoma or CRC, and identifying a need for a colonoscopy of the subject based on the classified risk of advanced adenoma or CRC.

In various embodiments of the method is provided for identifying and managing a subject at risk of an advanced adenoma or CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the advanced adenoma or CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the advanced adenoma or CRC disease state.

In various embodiments, the method further comprises identifying a need for a colonoscopy of the subject based on the classified risk of advanced adenoma or CRC when the disease indicator falls above a risk threshold.

In various embodiments, the method further comprises identifying a need for a colonoscopy of the subject based on the classified risk of advanced adenoma or CRC when the disease indicator falls above the selected threshold.

In various embodiments, the disease indicator comprises a risk score, and the method further comprises identifying a need for a colonoscopy of the subject based on the classified risk of advanced adenoma or CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

### VI.C.2 Monitoring a Subject for an Advanced Adenoma or Colorectal Cancer Disease State-Based on Table 2

FIG. 7B is a flowchart of a process for monitoring a subject for an advanced adenoma or Colorectal Cancer (CRC) disease state in accordance with one or more embodiments. Process 750 may be implemented using, for example, at least a portion of workflow 100 as described in FIGS. 1, 2, and/or analysis system 300 as described in FIG. 3.

Step 752 includes receiving first peptide structure data for a first biological sample obtained from a subject at a first timepoint.

Step 754 includes analyzing the first peptide structure data using a supervised machine learning model to generate a first disease indicator based on at least 1 peptide structure selected from a group of peptide structures identified in Table 2. The group of peptide structures in Table 2 includes a group of peptide structures associated with an advanced adenoma or CRC disease state in accordance with various embodiments. The supervised machine can be a binary classification model. In some embodiments, the binary classification model can be a logistical regression model.

Step 756 includes receiving second peptide structure data of a second biological sample obtained from the subject at a second timepoint.

Step 758 includes analyzing the second peptide structure data using the supervised machine learning model to generate a second disease indicator based on the at least 1 peptide structure selected from the group of peptide structures identified in Table 2.

Step 760 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the advanced adenoma or CRC disease state and the second biological sample evidences the positive diagnosis for the advanced adenoma or CRC disease. In other embodiments, the diagnosis output identifies whether a non-adenoma or non-CRC disease state has progressed to the advanced adenoma or CRC disease state, respectively, wherein the non-adenoma or non-CRC disease state includes either a healthy state, or a control state.

In accordance with various embodiments, a method is provided for identifying and managing a subject at risk of an advanced adenoma or CRC disease state. The method can comprise receiving a biological sample from the subject, determining a quantity of at least 1 peptide structure identified in Table 2 in the biological sample, analyzing the quantity of each peptide structure using at least one machine learning model to generate a disease indicator, generating a diagnosis output based on the disease indicator that classifies the biological sample as evidencing that the subject has a risk for advanced adenoma or CRC, and identifying a need for a colonoscopy of the subject based on the classified risk of advanced adenoma or CRC.

In various embodiments of the method is provided for identifying and managing a subject at risk of an advanced adenoma or CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the advanced adenoma or CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the advanced adenoma or CRC disease state.

In various embodiments, the method further comprises identifying a need for a colonoscopy of the subject based on the classified risk of advanced adenoma or CRC when the disease indicator falls above a risk threshold.

In various embodiments, the method further comprises identifying a need for a colonoscopy of the subject based on the classified risk of advanced adenoma or CRC when the disease indicator falls above the selected threshold.

In various embodiments, the disease indicator comprises a risk score, and the method further comprises identifying a need for a colonoscopy of the subject based on the classified risk of advanced adenoma or CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

### VII.A Peptide Structure and Product Ion Compositions, Kits and Reagents Based on Table 1

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 1-31, listed in Table 1.

Aspects of the disclosure include compositions comprising one or more precursor ions having a defined charge and/or defined mass-to-charge (m/z) ratio, as listed in Table 3A. Aspects of the disclosure include compositions comprising one or more product ions having a defined mass-to-charge (m/z) ratio, which product ions are produced by converting a peptide structure described herein (e.g., a peptide structure listed in Table 1 and 4A) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 3A, having an m/z ratio selected from the list provided for each peptide structure in Table 1 and/or Table 4A.

In some embodiments, a composition comprises at least one of peptide structures of PS-ID No's. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 identified in Table 1. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or all 31 of the peptide structures of PS-ID No's. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 identified in Table 1.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or all 31 of the peptide structures of PS-ID No's. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 identified in Table 3A.

In some embodiments, a composition comprises a peptide structure or a product ion. The peptide structure or product ion comprises an amino acid sequence having at least 90% sequence identity to any one of SEQ ID NOS: 1-31, as identified in Tables 4A and/or 4B, corresponding to peptide structures PS-ID No's 1-31 in Table 1.

In some embodiments, the product ion is selected as one from a group consisting of product ions identified in Table 3A, including product ions falling within an identified m/z range of the m/z ratio identified in Table 3A and characterized as having a precursor ion having an m/z ratio within an identified m/z range of the m/z ratio identified in Table 3A. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 3A, and characterized as having a precursor ion having an m/z ratio that falls within at least one of first range (±0.5), a second range (±1.0), or a third range (±1.0) of the precursor ion m/z ratio identified in Table 3A.

TABLE 3A
Mass Spectrometry-Related Characteristics for the Peptide Structures
associated with AA or CRC in accordance with Table 1

| | 1st | 1st | 2nd | 2nd | 1^{st} | 2nd | | Collision |
|---|---|---|---|---|---|---|---|---|
| PS-ID | Precursor | Precursor | Precursor | Precursor | Product | Product | RT - | Energy - |
| No. | m/z | charge | m/z | charge | m/z | m/z | min | V |
| 1 | 1224.5 | 3 | N/A | N/A | 366.1 | 980 | 37.5 | 30 |
| 2 | 1027.7 | 4 | N/A | N/A | 366.1 | 980 | 38 | 25 |
| 3 | 851.1 | 4 | 1134.5 | 3 | 366.1 | 1398.6 | 11.4 | 20 |
| 4 | 1323.1 | 4 | N/A | N/A | 366.1 | N/A | 23.1 | 33 |
| 5 | 1220.1 | 3 | N/A | N/A | 274.1 | 999.4 | 5.4 | 35 |
| 6 | 891 | 5 | N/A | N/A | 366.1 | 913.5 | 23.9 | 15 |
| 7 | 1019.2 | 4 | N/A | N/A | 366.1 | N/A | 40.8 | 20 |
| 8 | 1133.5 | 4 | N/A | N/A | 366.1 | N/A | 40.9 | 23 |
| 9 | 749.8 | 2 | N/A | N/A | 827.4 | 642.4 | 19.1 | 22 |
| 10 | 626.2 | 2 | N/A | N/A | 491.2 | 780.3 | 19.9 | 20 |
| 11 | 962.4 | 4 | N/A | N/A | 366.1 | N/A | 30 | 20 |
| 12 | 820.9 | 2 | N/A | N/A | 1052.5 | 678.3 | 31.5 | 25 |
| 13 | 993.1 | 3 | N/A | N/A | 366.1 | N/A | 6.1 | 25 |
| 14 | 1212.2 | 3 | 1211.9 | 3 | 1230.1 | 366.1 | 27.2 | 35 |
| 15 | 1196 | 4 | N/A | N/A | 366.1 | 274.1 | 27.6 | 30 |
| 16 | 1127.2 | 4 | N/A | N/A | 366.1 | N/A | 14.2 | 30 |
| 17 | 1314.9 | 3 | N/A | N/A | 366.1 | N/A | 30.6 | 30 |
| 18 | 1184.5 | 4 | N/A | N/A | 204.1 | N/A | 40.5 | 35 |
| 19 | 1001.2 | 4 | N/A | N/A | 366.1 | N/A | 30.4 | 24 |
| 20 | 1128.8 | 4 | N/A | N/A | 366.1 | N/A | 30 | 28 |
| 21 | 1124 | 5 | N/A | N/A | 366.1 | N/A | 33.2 | 20 |
| 22 | 1039.1 | 3 | N/A | N/A | 366.1 | 1112.5 | 10.2 | 25 |
| 23 | 990.7 | 3 | N/A | N/A | 366.1 | 1112.5 | 9.8 | 25 |
| 24 | 1114.2 | 4 | N/A | N/A | 204.1 | 1152.6 | 35.4 | 30 |
| 25 | 1216.5 | 2 | N/A | N/A | 204.1 | N/A | 7.9 | 35 |
| 26 | 879 | 3 | N/A | N/A | 204.1 | 1392.6 | 7.9 | 21 |
| 27 | 1069.2 | 4 | N/A | N/A | 204.1 | N/A | 39.8 | 25 |
| 28 | 1143.1 | 3 | N/A | N/A | 274.1 | 712.4 | 23.71 | 25 |
| 29 | 1181.1 | 4 | N/A | N/A | 366.1 | N/A | 32.7 | 29 |
| 30 | 1177 | 4 | N/A | N/A | 366.1 | N/A | 35.2 | 29 |
| 31 | 1039.4 | 3 | N/A | N/A | 366.1 | 1114.6 | 24.8 | 32 |

Table 4A defines the peptide sequences or list of amino acids for SEQ ID NOS: 1-31 from Table 1.

TABLE 4A
Peptide Sequences in accordance with Table 1

| PS-ID | | Pept. |
|---|---|---|
| No. | Peptide Sequence | SEQ ID NO |
| 1 | YLGNATAIFFLPDEGK | 1 |
| 2 | YLGNATAIFFLPDEGK | 2 |
| 3 | DIENFNSTQK | 3 |
| 4 | QDQCIYNTTYLNVQR | 4 |
| 5 | NEEYNK | 5 |
| 6 | VYIHPFHLVIHNESTCEQLAK | 6 |
| 7 | LGACNDTLQQLMEVFK | 7 |
| 8 | SLTFNETYQDISELVYGAK | 8 |
| 9 | AATVGSLAGQPLQER | 9 |
| 10 | LGNWSAMPSCK | 10 |
| 11 | AGLQAFFQVQECNK | 11 |
| 12 | AGLQAFFQVQECNK | 12 |
| 13 | NGTAVCATNR | 13 |
| 14 | TVLTPATNHMGNVTFTIPANR | 14 |
| 15 | VCQDCPLLAPLNDTR | 15 |
| 16 | HEEGHMLNCTCFGQGR | 16 |
| 17 | ALPQPQNVTSLLGCTH | 17 |
| 18 | CSDGWSFDATTLDDNGTMLFFK | 18 |
| 19 | VVLHPNYSQVDIGLIK | 19 |
| 20 | VVLHPNYSQVDIGLIK | 20 |
| 21 | MVSHHNLTTGATLINEQWLLTTAK | 21 |
| 22 | DTFVNASR | 22 |
| 23 | DTFVNASR | 23 |
| 24 | VLSNNSDANLELINTWVAK | 24 |
| 25 | EEQYNSTYR | 25 |
| 26 | EEQYNSTYR | 26 |
| 27 | IVGGTNSSWGEWPWQVSLQVK | 27 |
| 28 | NFTENDLLVR | 28 |
| 29 | QQQHLFGSNVTDCSGNFCLFR | 29 |
| 30 | QQQHLFGSNVTDCSGNFCLFR | 30 |
| 31 | NGSLFAFR | 31 |

Table 4B provides the start position of the peptide sequence within the protein sequence and the end position of the peptide sequence within the protein sequence.

TABLE 4B
Markers and Protein Positions in accordance with Table 1

| PS- | | | Start | End |
|---|---|---|---|---|
| ID No. | PS-NAME | Peptide Sequence | Position | Position |
| 1 | A1AT_271_5401 | YLGNATAIFFLPDEGK | 268 | 283 |
| | | (SEQ ID NO: 1) | | |
| 2 | A1AT_271_5412 | YLGNATAIFFLPDEGK | 268 | 283 |
| | | (SEQ ID NO: 2) | | |
| 3 | AFAM_33_5402 | DIENFNSTQK | 28 | 37 |
| | | (SEQ ID NO: 3) | | |
| 4 | AGP1_93_7613 | QDQCIYNTTYLNVQR | 87 | 101 |
| | | (SEQ ID NO: 4) | | |
| 5 | AGP12_56_6503 | NEEYNK | 52 | 57 |
| | | (SEQ ID NO: 5) | | |
| 6 | ANGT_47_5401 | VYIHPFHLVIHNESTCEQLAK | 36 | 56 |
| | | (SEQ ID NO: 6) | | |
| 7 | ANT_128_5402 | LGACNDTLQQLMEVFK | 124 | 139 |
| | | (SEQ ID NO: 7) | | |
| 8 | ANT_187_5412 | SLTFNETYQDISELVYGAK | 183 | 201 |
| | | (SEQ ID NO: 8) | | |
| 9 | APOE_212_NONGLY- | AATVGSLAGQPLQER | 210 | 224 |
| | COSYLATED | (SEQ ID NO: 9) | | |
| 10 | APOH_253_NONGLY- | LGNWSAMPSCK | 251 | 261 |
| | COSYLATED | (SEQ ID NO: 10) | | |
| 11 | CERU_358_5402 | AGLQAFFQVQECNK | 346 | 359 |
| | | (SEQ ID NO: 11) | | |
| 12 | CERU_358_NONGLY- | AGLQAFFQVQECNK | 346 | 359 |
| | COSYLATED | (SEQ ID NO: 12) | | |
| 13 | CFAI_70_5401 | NGTAVCATNR | 70 | 79 |
| | | (SEQ ID NO: 13) | | |
| 14 | CO3_85_6200 | TVLTPATNHMGNVTFTIPANR | 74 | 94 |
| | | (SEQ ID NO: 14) | | |
| 15 | FETUA_156_6513 | VCQDCPLLAPLNDTR | 145 | 159 |
| | | (SEQ ID NO: 15) | | |
| 16 | FINC_542_6502 | HEEGHMLNCTCFGQGR | 535 | 550 |
| | | (SEQ ID NO: 16) | | |
| 17 | HEMO_453_5402 | ALPQPQNVTSLLGCTH | 447 | 462 |
| | | (SEQ ID NO: 17) | | |
| 18 | HEMO_64_5402 | CSDGWSFDATTLDDNGTMLFFK | 50 | 71 |
| | | (SEQ ID NO: 18) | | |
| 19 | HPT_241_5402 | VVLHPNYSQVDIGLIK | 236 | 251 |
| | | (SEQ ID NO: 19) | | |
| 20 | HPT_241_6512 | VVLHPNYSQVDIGLIK | 236 | 251 |
| | | (SEQ ID NO: 20) | | |
| 21 | HPT_184_7602 | MVSHHNLTTGATLINEQWLLTTAK | 179 | 202 |
| | | (SEQ ID NO: 21) | | |
| 22 | IC1_238_5402 | DTFVNASR | 234 | 241 |
| | | (SEQ ID NO: 22) | | |
| 23 | IC1_238_5411 | DTFVNASR | 234 | 241 |
| | | (SEQ ID NO: 23) | | |
| 24 | IC1_253_5412 | VLSNNSDANLELINTWVAK | 250 | 268 |
| | | (SEQ ID NO: 24) | | |
| 25 | IGG1_297_3310 | EEQYNSTYR | 176 | 184 |
| | | (SEQ ID NO: 25) | | |
| 26 | IGG1_297_3410 | EEQYNSTYR | 176 | 184 |
| | | (SEQ ID NO: 26) | | |
| 27 | KLKB1_396_5401 | IVGGTNSSWGEWPWQVSLQVK | 391 | 411 |
| | | (SEQ ID NO: 27) | | |
| 28 | THRB_416_5402 | NFTENDLLVR | 416 | 425 |
| | | (SEQ ID NO: 28) | | |
| 29 | TRFE_630_5402 | QQQHLFGSNVTDCSGNFCLFR | 622 | 642 |
| | | (SEQ ID NO: 29) | | |
| 30 | TRFE_630_5402_NH3loss | QQQHLFGSNVTDCSGNFCLFR | 622 | 642 |
| | | (SEQ ID NO: 30) | | |
| 31 | VTNC_169_5402 | NGSLFAFR | 169 | 176 |
| | | (SEQ ID NO: 31) | | |

### VII.B Peptide Structure and Product Ion Compositions, Kits and Reagents Based on Table 2

Aspects of the disclosure include compositions comprising one or more of the peptide structures listed in Table 2. In some embodiments, a composition comprises a plurality of the peptide structures listed in Table 2. In some embodiments, a composition comprises 1, 2, 3, 4, 5, 6, 7, 8, 9, 10, 11, 12, 13, 14, 15, 16, 17, 18, 19, 20, or all of the peptide structures listed in Table 2. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 3-9, 12, 14-16, 18, 25-28, and 31-35, listed in Table 2.

Aspects of the disclosure include compositions comprising one or more precursor ions having a defined charge and/or defined mass-to-charge (m/z) ratio, as listed in Table 3B. Aspects of the disclosure include compositions comprising one or more product ions having a defined mass-to-charge (m/z) ratio, which product ions are produced by converting a peptide structure described herein (e.g., a peptide structure listed in Table 2 and 4C) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 2). In some embodiments, a composition comprises a set of the product ions listed in Table 3B, having an m/z ratio selected from the list provided for each peptide structure in Table 2 and/or Table 4C.

In some embodiments, a composition comprises at least one of peptide structures of PS-ID NO: 3-9, 12, 14-16, 18, 25-28, and 31-35 identified in Table 2. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 21 of the peptide structures of PS-ID NO: 3-9, 12, 14-16, 18, 25-28, and 31-35 identified in Table 2.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 21 of the peptide structures of PS-ID NO: 3-9, 12, 14-16, 18, 25-28, and 31-35 identified in Table 3B.

In some embodiments, a composition comprises a peptide structure or a product ion. The peptide structure or product ion comprises an amino acid sequence having at least 90% sequence identity to any one of SEQ ID NOS: 3-9, 12, 14-16, 18, 25-28, and 31-35, as identified in Tables 4C and/or 4D, corresponding to peptide structures PS-ID NO: 3-9, 12, 14-16, 18, 25-28, and 31-35 in Table 2.

In some embodiments, the product ion is selected as one from a group consisting of product ions identified in Table 3B, including product ions falling within an identified m/z range of the m/z ratio identified in Table 3B and characterized as having a precursor ion having an m/z ratio within an identified m/z range of the m/z ratio identified in Table 3B. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 3B, and characterized as having a precursor ion having an m/z ratio that falls within at least one of first range (±0.5), a second range (±1.0), or a third range (±1.0) of the precursor ion m/z ratio identified in Table 3B.

TABLE 3B
Mass Spectrometry-Related Characteristics for the 21 Peptide Structures
associated with AA or CRC in accordance with Table 2

| | 1st | 1st | 2nd | 2nd | 1^{st} | 2nd | | Collision |
|---|---|---|---|---|---|---|---|---|
| PS-ID | Precursor | Precursor | Precursor | Precursor | Product | Product | RT - | Energy - |
| No. | m/z | charge | m/z | charge | m/z | m/z | min | V |
| 32 | 1243.3 | 5 | N/A | N/A | 274.1 | N/A | 38.3 | 35 |
| 3 | 851.1 | 4 | 1134.5 | 3 | 366.1 | 1398.6 | 11.4 | 20 |
| 4 | 1323.1 | 4 | N/A | N/A | 366.1 | N/A | 23.1 | 33 |
| 5 | 1220.1 | 3 | N/A | N/A | 274.1 | 999.4 | 5.4 | 35 |
| 6 | 891 | 5 | N/A | N/A | 366.1 | 913.5 | 23.9 | 15 |
| 7 | 1019.2 | 4 | N/A | N/A | 366.1 | N/A | 40.8 | 20 |
| 8 | 1133.5 | 4 | N/A | N/A | 366.1 | N/A | 40.9 | 23 |
| 9 | 749.8 | 2 | N/A | N/A | 827.4 | 642.4 | 19.1 | 22 |
| 12 | 820.9 | 2 | N/A | N/A | 1052.5 | 678.3 | 31.5 | 25 |
| 33 | 1084.2 | 4 | N/A | N/A | 204.1 | N/A | 27.4 | 35 |
| 14 | 1212.2 | 3 | 1211.9 | 3 | 1230.1 | 366.1 | 27.2 | 35 |
| 15 | 1196 | 4 | N/A | N/A | 366.1 | 274.1 | 27.6 | 30 |
| 16 | 1127.2 | 4 | N/A | N/A | 366.1 | N/A | 14.2 | 30 |
| 18 | 1184.5 | 4 | N/A | N/A | 204.1 | N/A | 40.5 | 35 |
| 34 | 1201.5 | 4 | N/A | N/A | 366.1 | N/A | 30.9 | 30 |
| 25 | 1216.5 | 2 | N/A | N/A | 204.1 | N/A | 7.9 | 35 |
| 26 | 879 | 3 | N/A | N/A | 204.1 | 1392.6 | 7.9 | 21 |
| 35 | 946.5 | 3 | N/A | N/A | 204.1 | 1392.6 | 8.1 | 15 |
| 27 | 1069.2 | 4 | N/A | N/A | 204.1 | N/A | 39.8 | 25 |
| 28 | 1143.1 | 3 | N/A | N/A | 274.1 | 712.4 | 23.71 | 25 |
| 31 | 1039.4 | 3 | N/A | N/A | 366.1 | 1114.6 | 24.8 | 32 |

Table 4C defines the peptide sequences or list of amino acids for PS-ID NO: 3-9, 12, 14-16, 18, 25-28, and 31-35 from Table 2.

TABLE 4C
Peptide Sequences in accordance with Table 2

| | | Prot. | Pept. |
|---|---|---|---|
| PS-ID | | SEQ | SEQ |
| No. | Peptide Sequence | ID NO | ID NO |
| 32 | FNLTETSEAEIHQSFQHLLR | 57 | 32 |
| | (SEQ ID NO: 32) | | |
| 3 | DIENFNSTQK | 37 | 3 |
| | (SEQ ID NO: 3) | | |
| 4 | QDQCIYNTTYLNVQR | 38 | 4 |
| | (SEQ ID NO: 4) | | |
| 5 | NEEYNK | 38 or 39 | 5 |
| | (SEQ ID NO: 5) | | |
| 6 | VYIHPFHLVIHNESTCEQLAK | 40 | 6 |
| | (SEQ ID NO: 6) | | |
| 7 | LGACNDTLQQLMEVFK | 41 | 7 |
| | (SEQ ID NO: 7) | | |
| 8 | SLTFNETYQDISELVYGAK | 41 | 8 |
| | (SEQ ID NO: 8) | | |
| 9 | AATVGSLAGQPLQER | 42 | 9 |
| | (SEQ ID NO: 9) | | |
| 12 | AGLQAFFQVQECNK | 44 | 12 |
| | (SEQ ID NO: 12) | | |
| 33 | ENLTAPGSDSAVFFEQGTTR | 44 | 33 |
| | (SEQ ID NO: 33) | | |
| 14 | TVLTPATNHMGNVTFTIPANR | 46 | 14 |
| | (SEQ ID NO: 14) | | |
| 15 | VCQDCPLLAPLNDTR | 47 | 15 |
| | (SEQ ID NO: 15) | | |
| 16 | HEEGHMLNCTCFGQGR | 48 | 16 |
| | (SEQ ID NO: 16) | | |
| 18 | CSDGWSFDATTLDDNGTMLFFK | 49 | 18 |
| | (SEQ ID NO: 18) | | |
| 34 | VVLHPNYSQVDIGLIK | 50 | 34 |
| | (SEQ ID NO: 34) | | |
| 25 | EEQYNSTYR | 52 | 25 |
| | (SEQ ID NO: 25) | | |
| 26 | EEQYNSTYR | 52 | 26 |
| | (SEQ ID NO: 26) | | |
| 35 | EEQYNSTYR | 52 | 35 |
| | (SEQ ID NO: 35) | | |
| 27 | IVGGTNSSWGEWPWQVSLQVK | 53 | 27 |
| | (SEQ ID NO: 27) | | |
| 28 | NFTENDLLVR | 54 | 28 |
| | (SEQ ID NO: 28) | | |
| 31 | NGSLFAFR | 56 | 31 |
| | (SEQ ID NO: 31) | | |

TABLE 4D
Markers and Protein Positions in accordance with Table 2

| PS- | | | Start | End |
|---|---|---|---|---|
| ID No. | PS-NAME | Peptide Sequence | Position | Position |
| 32 | AACT_106_7624 | FNLTETSEAEIHQSFQHLLR | 105 | 124 |
| | | (SEQ ID NO: 32) | | |
| 3 | AFAM_33_5402 | DIENFNSTQK | 28 | 37 |
| | | (SEQ ID NO: 3) | | |
| 4 | AGP1_93_7613 | QDQCIYNTTYLNVQR | 87 | 101 |
| | | (SEQ ID NO: 4) | | |
| 5 | AGP12_56_6503 | NEEYNK | 52 | 57 |
| | | (SEQ ID NO: 5) | | |
| 6 | ANGT_47_5401 | VYIHPFHLVIHNESTCEQLAK | 36 | 56 |
| | | (SEQ ID NO: 6) | | |
| 7 | ANT_128_5402 | LGACNDTLQQLMEVFK | 124 | 139 |
| | | (SEQ ID NO: 7) | | |
| 8 | ANT_187_5412 | SLTFNETYQDISELVYGAK | 183 | 201 |
| | | (SEQ ID NO: 8) | | |
| 9 | APOE_212_NONGLY- | AATVGSLAGQPLQER | 210 | 224 |
| | COSYLATED | (SEQ ID NO: 9) | | |
| 12 | CERU_358_NONGLY- | AGLQAFFQVQECNK | 346 | 359 |
| | COSYLATED | (SEQ ID NO: 12) | | |
| 33 | CERU_397_5402 | ENLTAPGSDSAVFFEQGTTR | 396 | 415 |
| | | (SEQ ID NO: 33) | | |
| 14 | CO3_85_6200 | TVLTPATNHMGNVTFTIPANR | 74 | 94 |
| | | (SEQ ID NO: 14) | | |
| 15 | FETUA_156_6513 | VCQDCPLLAPLNDTR | 145 | 159 |
| | | (SEQ ID NO: 15) | | |
| 16 | FINC_542_6502 | HEEGHMLNCTCFGQGR | 535 | 550 |
| | | (SEQ ID NO: 16) | | |
| 18 | HEMO_64_5402 | CSDGWSFDATTLDDNGTMLFFK | 50 | 71 |
| | | (SEQ ID NO: 18) | | |
| 34 | HPT_241_6513 | VVLHPNYSQVDIGLIK | 236 | 251 |
| | | (SEQ ID NO: 34) | | |
| 25 | IGG1_297_3310 | EEQYNSTYR | 176 | 184 |
| | | (SEQ ID NO: 25) | | |
| 26 | IGG1_297_3410 | EEQYNSTYR | 176 | 184 |
| | | (SEQ ID NO: 26) | | |
| 35 | IGG1_297_3510 | EEQYNSTYR | 176 | 184 |
| | | (SEQ ID NO: 35) | | |
| 27 | KLKB1_396_5401 | IVGGTNSSWGEWPWQVSLQVK | 391 | 411 |
| | | (SEQ ID NO: 27) | | |
| 28 | THRB_416_5402 | NFTENDLLVR | 416 | 425 |
| | | (SEQ ID NO: 28) | | |
| 31 | VTNC_169_5402 | NGSLFAFR | 169 | 176 |
| | | (SEQ ID NO: 31) | | |

Table 5 identifies the proteins and the associated amino acid sequences for Table 1 and Table 2, and Table 8B. Table 5 identifies a corresponding protein abbreviation and protein name for each of protein SEQ ID NOS: 36-57, 101, and 102. Further, Table 5 identifies a corresponding Uniprot ID for each of protein SEQ ID NOS: 36-57, 101, and 102.

TABLE 5
Sequence of Amino Acids for Proteins Corresponding to Table 1, Table 2, and Table 8B

| SEQ | Protein | | Uniprot | |
|---|---|---|---|---|
| ID NO | Abbreviation | Protein Name | ID | Protein Sequence |
| 36 | A1AT | Alpha-1- | P01009 | MPSSVSWGILLLAGLCCLVPVSLAEDPQGDAAQKTDTSHHDQDHPT |
| | | antitrypsin | | FNKITPNLAEFAFSLYRQLAHQSNSTNIFFSPVSIATAFAMLSLGTKAD |
| | | | | THDEILEGLNFNLTEIPEAQIHEGFQELLRTLNQPDSQLQLTTGNGLFL |
| | | | | SEGLKLVDKFLEDVKKLYHSEAFTVNFGDTEEAKKQINDYVEKGTQGK |
| | | | | IVDLVKELDRDTVFALVNYIFFKGKWERPFEVKDTEEEDFHVDQVTTV |
| | | | | KVPMMKRLGMFNIQHCKKLSSWVLLMKYLGNATAIFFLPDEGKLQH |
| | | | | LENELTHDIITKFLENEDRRSASLHLPKLSITGTYDLKSVLGQLGITKVFS |
| | | | | NGADLSGVTEEAPLKLSKAVHKAVLTIDEKGTEAAGAMFLEAIPMSIP |
| | | | | PEVKFNKPFVFLMIEQNTKSPLFMGKVVNPTQK |
| 37 | AFAM | Afamin | P43652 | MKLLKLTGFIFFLFFLTESLTLPTQPRDIENFNSTQKFIEDNIEYITIIAFA |
| | | | | QYVQEATFEEMEKLVKDMVEYKDRCMADKTLPECSKLPNNVLQEKI |
| | | | | CAMEGLPQKHNFSHCCSKVDAQRRLCFFYNKKSDVGFLPPFPTLDPE |
| | | | | EKCQAYESNRESLLNHFLYEVARRNPFVFAPTLLTVAVHFEEVAKSCC |
| | | | | EEQNKVNCLQTRAIPVTQYLKAFSSYQKHVCGALLKFGTKVVHFIYIAI |
| | | | | LSQKFPKIEFKELISLVEDVSSNYDGCCEGDVVQCIRDTSKVMNHICSK |
| | | | | QDSISSKIKECCEKKIPERGQCIINSNKDDRPKDLSLREGKFTDSENVC |
| | | | | QERDADPDTFFAKFTFEYSRRHPDLSIPELLRIVQIYKDLLRNCQNTEN |
| | | | | PPGCYRYAEDKFNETTEKSLKMVQQECKHFQNLGKDGLKYHYLIRLT |
| | | | | KIAPQLSTEELVSLGEKMVTAFTTCCTLSEEFACVDNLADLVFGELCG |
| | | | | VNENRTINPAVDHCCKTNFAFRRPCFESLKADKTYVPPPFSQDLFTFH |
| | | | | ADMCQSQNEELQRKTDRFLVNLVKLKHELTDEELQSLFTNFANVVD |
| | | | | KCCKAESPEVCFNEESPKIGN |
| 38 | AGP1 | Alpha-1-acid | P02763 | MALSWVLTVLSLLPLLEAQIPLCANLVPVPITNATLDRITGKWFYIASA |
| | | glycoprotein | | FRNEEYNKSVQEIQATFFYFTPNKTEDTIFLREYQTRQDQCIYNTTYLN |
| | | 1 | | VQRENGTISRYVGGQEHFAHLLILRDTKTYMLAFDVNDEKNWGLSV |
| | | | | YADKPETTKEQLGEFYEALDCLRIPKSDVVYTDWKKDKCEPLEKQHEK |
| | | | | ERKQEEGES |
| 39 | AGP2 | Alpha-1-acid | P19652 | MALSWVLTVLSLLPLLEAQIPLCANLVPVPITNATLDRITGKWFYIASA |
| | | glycoprotein | | FRNEEYNKSVQEIQATFFYFTPNKTEDTIFLREYQTRQNQCFYNSSYL |
| | | 2 | | NVQRENGTVSRYEGGREHVAHLLFLRDTKTLMFGSYLDDEKNWGLS |
| | | | | FYADKPETTKEQLGEFYEALDCLCIPRSDVMYTDWKKDKCEPLEKQH |
| | | | | EKERKQEEGES |
| 40 | ANGT | Angiotensino | P01019 | MRKRAPQSEMAPAGVSLRATILCLLAWAGLAAGDRVYIHPFHLVIH |
| | | gen | | NESTCEQLAKANAGKPKDPTFIPAPIQAKTSPVDEKALQDQLVLVAA |
| | | | | KLDTEDKLRAAMVGMLANFLGFRIYGMHSELWGVVHGATVLSPTA |
| | | | | VFGTLASLYLGALDHTADRLQAILGVPWKDKNCTSRLDAHKVLSALQ |
| | | | | AVQGLLVAQGRADSQAQLLLSTVVGVFTAPGLHLKQPFVQGLALYT |
| | | | | PVVLPRSLDFTELDVAAEKIDRFMQAVTGWKTGCSLMGASVDSTLA |
| | | | | FNTYVHFQGKMKGFSLLAEPQEFWVDNSTSVSVPMLSGMGTFQH |
| | | | | WSDIQDNFSVTQVPFTESACLLLIQPHYASDLDKVEGLTFQQNSLNW |
| | | | | MKKLSPRTIHLTMPQLVLQGSYDLQDLLAQAELPAILHTELNLQKLSN |
| | | | | DRIRVGEVLNSIFFELEADEREPTESTQQLNKPEVLEVTLNRPFLFAVY |
| | | | | DQSATALHFLGRVANPLSTA |
| 41 | ANT | Antithrombin | P01008 | MYSNVIGTVTSGKRKVYLLSLLLIGFWDCVTCHGSPVDICTAKPRDIP |
| | | | | MNPMCIYRSPEKKATEDEGSEQKIPEATNRRVWELSKANSRFATTFY |
| | | | | QHLADSKNDNDNIFLSPLSISTAFAMTKLGACNDTLQQLMEVFKFDT |
| | | | | ISEKTSDQIHFFFAKLNCRLYRKANKSSKLVSANRLFGDKSLTFNETYQ |
| | | | | DISELVYGAKLQPLDFKENAEQSRAAINKWVSNKTEGRITDVIPSEAIN |
| | | | | ELTVLVLVNTIYFKGLWKSKFSPENTRKELFYKADGESCSASMMYQE |
| | | | | GKFRYRRVAEGTQVLELPFKGDDITMVLILPKPEKSLAKVEKELTPEVL |
| | | | | QEWLDELEEMMLVVHMPRFRIEDGFSLKEQLQDMGLVDLFSPEKSK |
| | | | | LPGIVAEGRDDLYVSDAFHKAFLEVNEEGSEAAASTAVVIAGRSLNPN |
| | | | | RVTFKANRPFLVFIREVPLNTIIFMGRVANPCVK |
| 42 | APOE | Apolipoprote | P02649 | MKVLWAALLVTFLAGCQAKVEQAVETEPEPELRQQTEWQSGQRW |
| | | in E | | ELALGRFWDYLRWVQTLSEQVQEELLSSQVTQELRALMDETMKELK |
| | | | | AYKSELEEQLTPVAEETRARLSKELQAAQARLGADMEDVCGRLVQYR |
| | | | | GEVQAMLGQSTEELRVRLASHLRKLRKRLLRDADDLQKRLAVYQAG |
| | | | | AREGAERGLSAIRERLGPLVEQGRVRAATVGSLAGQPLQERAQAWG |
| | | | | ERLRARMEEMGSRTRDRLDEVKEQVAEVRAKLEEQAQQIRLQAEAF |
| | | | | QARLKSWFEPLVEDMQRQWAGLVEKVQAAVGTSAAPVPSDNH |
| 43 | APOH | Beta-2- | P02749 | MISPVLILFSSFLCHVAIAGRTCPKPDDLPFSTVVPLKTFYEPGEEITYSC |
| | | glycoprotein | | KPGYVSRGGMRKFICPLTGLWPINTLKCTPRVCPFAGILENGAVRYTT |
| | | 1 | | FEYPNTISFSCNTGFYLNGADSAKCTEEGKWSPELPVCAPIICPPPSIPT |
| | | | | FATLRVYKPSAGNNSLYRDTAVFECLPQHAMFGNDTITCTTHGNWT |
| | | | | KLPECREVKCPFPSRPDNGFVNYPAKPTLYYKDKATFGCHDGYSLDG |
| | | | | PEEIECTKLGNWSAMPSCKASCKVPVKKATVVYQGERVKIQEKFKNG |
| | | | | MLHGDKVSFFCKNKEKKCSYTEDAQCIDGTIEVPKCFKEHSSLAFWKT |
| | | | | DASDVKPC |
| 44 | CERU | Ceruloplasmi | P00450 | MKILILGIFLFLCSTPAWAKEKHYYIGIIETTWDYASDHGEKKLISVDTE |
| | | n | | HSNIYLQNGPDRIGRLYKKALYLQYTDETFRTTIEKPVWLGFLGPIIKAE |
| | | | | TGDKVYVHLKNLASRPYTFHSHGITYYKEHEGAIYPDNTTDFQRADD |
| | | | | KVYPGEQYTYMLLATEEQSPGEGDGNCVTRIYHSHIDAPKDIASGLIG |
| | | | | PLIICKKDSLDKEKEKHIDREFVVMFSVVDENFSWYLEDNIKTYCSEPE |
| | | | | KVDKDNEDFQESNRMYSVNGYTFGSLPGLSMCAEDRVKWYLFGM |
| | | | | GNEVDVHAAFFHGQALTNKNYRIDTINLFPATLFDAYMVAQNPGE |
| | | | | WMLSCQNLNHLKAGLQAFFQVQECNKSSSKDNIRGKHVRHYYIAAE |
| | | | | EIIWNYAPSGIDIFTKENLTAPGSDSAVFFEQGTTRIGGSYKKLVYREYT |
| | | | | DASFTNRKERGPEEEHLGILGPVIWAEVGDTIRVTFHNKGAYPLSIEPI |
| | | | | GVRFNKNNEGTYYSPNYNPQSRSVPPSASHVAPTETFTYEWTVPKE |
| | | | | VGPTNADPVCLAKMYYSAVDPTKDIFTGLIGPMKICKKGSLHANGRQ |
| | | | | KDVDKEFYLFPTVFDENESLLLEDNIRMFTTAPDQVDKEDEDFQESN |
| | | | | KMHSMNGFMYGNQPGLTMCKGDSVVWYLFSAGNEADVHGIYFS |
| | | | | GNTYLWRGERRDTANLFPQTSLTLHMWPDTEGTFNVECLTTDHYT |
| | | | | GGMKQKYTVNQCRRQSEDSTFYLGERTYYIAAVEVEWDYSPQREW |
| | | | | EKELHHLQEQNVSNAFLDKGEFYIGSKYKKVVYRQYTDSTFRVPVERK |
| | | | | AEEEHLGILGPQLHADVGDKVKIIFKNMATRPYSIHAHGVQTESSTVT |
| | | | | PTLPGETLTYVWKIPERSGAGTEDSACIPWAYYSTVDQVKDLYSGLIG |
| | | | | PLIVCRRPYLKVFNPRRKLEFALLFLVFDENESWYLDDNIKTYSDHPEK |
| | | | | VNKDDEEFIESNKMHAINGRMFGNLQGLTMHVGDEVNWYLMGM |
| | | | | GNEIDLHTVHFHGHSFQYKHRGVYSSDVFDIFPGTYQTLEMFPRTPG |
| | | | | IWLLHCHVTDHIHAGMETTYTVLQNEDTKSG |
| 45 | CFAI | Complement | P05156 | MKLLHVFLLFLCFHLRFCKVTYTSQEDLVEKKCLAKKYTHLSCDKVFCQ |
| | | Factor I | | PWQRCIEGTCVCKLPYQCPKNGTAVCATNRRSFPTYCQQKSLECLHP |
| | | | | GTKFLNNGTCTAEGKFSVSLKHGNTDSEGIVEVKLVDQDKTMFICKS |
| | | | | SWSMREANVACLDLGFQQGADTQRRFKLSDLSINSTECLHVHCRGL |
| | | | | ETSLAECTFTKRRTMGYQDFADVVCYTQKADSPMDDFFQCVNGKYI |
| | | | | SQMKACDGINDCGDQSDELCCKACQGKGFHCKSGVCIPSQYQCNG |
| | | | | EVDCITGEDEVGCAGFASVTQEETEILTADMDAERRRIKSLLPKLSCG |
| | | | | VKNRMHIRRKRIVGGKRAQLGDLPWQVAIKDASGITCGGIYIGGCWI |
| | | | | LTAAHCLRASKTHRYQIWTTVVDWIHPDLKRIVIEYVDRIIFHENYNA |
| | | | | GTYQNDIALIEMKKDGNKKDCELPRSIPACVPWSPYLFQPNDTCIVS |
| | | | | GWGREKDNERVFSLQWGEVKLISNCSKFYGNRFYEKEMECAGTYD |
| | | | | GSIDACKGDSGGPLVCMDANNVTYVWGVVSWGENCGKPEFPGVY |
| | | | | TKVANYFDWISYHVGRPFISQYNV |
| 46 | CO3 | Complement | P01024 | MGPTSGPSLLLLLLTHLPLALGSPMYSIITPNILRLESEETMVLEAHDA |
| | | C3 | | QGDVPVTVTVHDFPGKKLVLSSEKTVLTPATNHMGNVTFTIPANREF |
| | | | | KSEKGRNKFVTVQATFGTQVVEKVVLVSLQSGYLFIQTDKTIYTPGST |
| | | | | VLYRIFTVNHKLLPVGRTVMVNIENPEGIPVKQDSLSSQNQLGVLPLS |
| | | | | WDIPELVNMGQWKIRAYYENSPQQVFSTEFEVKEYVLPSFEVIVEPT |
| | | | | EKFYYIYNEKGLEVTITARFLYGKKVEGTAFVIFGIQDGEQRISLPESLKR |
| | | | | IPIEDGSGEVVLSRKVLLDGVQNPRAEDLVGKSLYVSATVILHSGSDM |
| | | | | VQAERSGIPIVTSPYQIHFTKTPKYFKPGMPFDLMVFVTNPDGSPAYR |
| | | | | VPVAVQGEDTVQSLTQGDGVAKLSINTHPSQKPLSITVRTKKQELSE |
| | | | | AEQATRTMQALPYSTVGNSNNYLHLSVLRTELRPGETLNVNFLLRM |
| | | | | DRAHEAKIRYYTYLIMNKGRLLKAGRQVREPGQDLVVLPLSITTDFIPS |
| | | | | FRLVAYYTLIGASGQREVVADSVWVDVKDSCVGSLVVKSGQSEDRQ |
| | | | | PVPGQQMTLKIEGDHGARVVLVAVDKGVFVLNKKNKLTQSKIWDV |
| | | | | VEKADIGCTPGSGKDYAGVFSDAGLTFTSSSGQQTAQRAELQCPQP |
| | | | | AARRRRSVQLTEKRMDKVGKYPKELRKCCEDGMRENPMRFSCQRR |
| | | | | TRFISLGEACKKVELDCCNYITELRRQHARASHLGLARSNLDEDIIAEE |
| | | | | NIVSRSEFPESWLWNVEDLKEPPKNGISTKLMNIFLKDSITTWEILAVS |
| | | | | MSDKKGICVADPFEVTVMQDFFIDLRLPYSVVRNEQVEIRAVLYNYR |
| | | | | QNQELKVRVELLHNPAFCSLATTKRRHQQTVTIPPKSSLSVPYVIVPLK |
| | | | | TGLQEVEVKAAVYHHFISDGVRKSLKVVPEGIRMNKTVAVRTLDPER |
| | | | | LGREGVQKEDIPPADLSDQVPDTESETRILLQGTPVAQMTEDAVDAE |
| | | | | RLKHLIVTPSGCGEQNMIGMTPTVIAVHYLDETEQWEKFGLEKRQG |
| | | | | ALELIKKGYTQQLAFRQPSSAFAAFVKRAPSTWLTAYVVKVFSLAVNL |
| | | | | IAIDSQVLCGAVKWLILEKQKPDGVFQEDAPVIHQEMIGGLRNNNEK |
| | | | | DMALTAFVLISLQEAKDICEEQVNSLPGSITKAGDFLEANYMNLQRSY |
| | | | | TVAIAGYALAQMGRLKGPLLNKFLTTAKDKNRWEDPGKQLYNVEAT |
| | | | | SYALLALLQLKDFDFVPPVVRWLNEQRYYGGGYGSTQATFMVFQAL |
| | | | | AQYQKDAPDHQELNLDVSLQLPSRSSKITHRIHWESASLLRSEETKEN |
| | | | | EGFTVTAEGKGQGTLSVVTMYHAKAKDQLTCNKFDLKVTIKPAPETE |
| | | | | KRPQDAKNTMILEICTRYRGDQDATMSILDISMMTGFAPDTDDLKQ |
| | | | | LANGVDRYISKYELDKAFSDRNTLIIYLDKVSHSEDDCLAFKVHQYFNV |
| | | | | ELIQPGAVKVYAYYNLEESCTRFYHPEKEDGKLNKLCRDELCRCAEEN |
| | | | | CFIQKSDDKVTLEERLDKACEPGVDYVYKTRLVKVQLSNDFDEYIMAI |
| | | | | EQTIKSGSDEVQVGQQRTFISPIKCREALKLEEKKHYLMWGLSSDFW |
| | | | | GEKPNLSYIIGKDTWVEHWPEEDECQDEENQKQCQDLGAFTESMV |
| | | | | VFGCPN |
| 47 | FETUA | Alpha-2-HS- | P02765 | MKSLVLLLCLAQLWGCHSAPHGPGLIYRQPNCDDPETEEAALVAIDYI |
| | | glycoprotein | | NQNLPWGYKHTLNQIDEVKVWPQQPSGELFEIEIDTLETTCHVLDPT |
| | | | | PVARCSVRQLKEHAVEGDCDFQLLKLDGKFSVVYAKCDSSPDSAEDV |
| | | | | RKVCQDCPLLAPLNDTRVVHAAKAALAAFNAQNNGSNFQLEEISRA |
| | | | | QLVPLPPSTYVEFTVSGTDCVAKEATEAAKCNLLAEKQYGFCKATLSE |
| | | | | KLGGAEVAVTCMVFQTQPVSSQPQPEGANEAVPTPVVDPDAPPSP |
| | | | | PLGAPGLPPAGSPPDSHVLLAAPPGHQLHRAHYDLRHTFMGVVSLG |
| | | | | SPSGEVSHPRKTRTVVQPSVGAAAGPVVPPCPGRIRHFKV |
| 48 | FINC | Fibronectin | P02751 | MLRGPGPGLLLLAVQCLGTAVPSTGASKSKRQAQQMVQPQSPVAV |
| | | | | SQSKPGCYDNGKHYQINQQWERTYLGNALVCTCYGGSRGFNCESKP |
| | | | | EAEETCFDKYTGNTYRVGDTYERPKDSMIWDCTCIGAGRGRISCTIA |
| | | | | NRCHEGGQSYKIGDTWRRPHETGGYMLECVCLGNGKGEWTCKPIA |
| | | | | EKCFDHAAGTSYVVGETWEKPYQGWMMVDCTCLGEGSGRITCTSR |
| | | | | NRCNDQDTRTSYRIGDTWSKKDNRGNLLQCICTGNGRGEWKCERH |
| | | | | TSVQTTSSGSGPFTDVRAAVYQPQPHPQPPPYGHCVTDSGVVYSVG |
| | | | | MQWLKTQGNKQMLCTCLGNGVSCQETAVTQTYGGNSNGEPCVLP |
| | | | | FTYNGRTFYSCTTEGRQDGHLWCSTTSNYEQDQKYSFCTDHTVLVQ |
| | | | | TRGGNSNGALCHFPFLYNNHNYTDCTSEGRRDNMKWCGTTQNYD |
| | | | | ADQKFGFCPMAAHEEICTTNEGVMYRIGDQWDKQHDMGHMMR |
| | | | | CTCVGNGRGEWTCIAYSQLRDQCIVDDITYNVNDTFHKRHEEGHML |
| | | | | NCTCFGQGRGRWKCDPVDQCQDSETGTFYQIGDSWEKYVHGVRY |
| | | | | QCYCYGRGIGEWHCQPLQTYPSSSGPVEVFITETPSQPNSHPIQWN |
| | | | | APQPSHISKYILRWRPKNSVGRWKEATIPGHLNSYTIKGLKPGVVYEG |
| | | | | QLISIQQYGHQEVTRFDFTTTSTSTPVTSNTVTGETTPFSPLVATSESV |
| | | | | TEITASSFVVSWVSASDTVSGFRVEYELSEEGDEPQYLDLPSTATSVNI |
| | | | | PDLLPGRKYIVNVYQISEDGEQSLILSTSQTTAPDAPPDTTVDQVDDT |
| | | | | SIVVRWSRPQAPITGYRIVYSPSVEGSSTELNLPETANSVTLSDLQPGV |
| | | | | QYNITIYAVEENQESTPVVIQQETTGTPRSDTVPSPRDLQFVEVTDVK |
| | | | | VTIMWTPPESAVTGYRVDVIPVNLPGEHGQRLPISRNTFAEVTGLSP |
| | | | | GVTYYFKVFAVSHGRESKPLTAQQTTKLDAPTNLQFVNETDSTVLVR |
| | | | | WTPPRAQITGYRLTVGLTRRGQPRQYNVGPSVSKYPLRNLQPASEYT |
| | | | | VSLVAIKGNQESPKATGVFTTLQPGSSIPPYNTEVTETTIVITWTPAPRI |
| | | | | GFKLGVRPSQGGEAPREVTSDSGSIVVSGLTPGVEYVYTIQVLRDGQ |
| | | | | ERDAPIVNKVVTPLSPPTNLHLEANPDTGVLTVSWERSTTPDITGYRI |
| | | | | TTTPTNGQQGNSLEEVVHADQSSCTFDNLSPGLEYNVSVYTVKDDK |
| | | | | ESVPISDTIIPEVPQLTDLSFVDITDSSIGLRWTPLNSSTIIGYRITVVAA |
| | | | | GEGIPIFEDFVDSSVGYYTVTGLEPGIDYDISVITLINGGESAPTTLTQQ |
| | | | | TAVPPPTDLRFTNIGPDTMRVTWAPPPSIDLTNFLVRYSPVKNEEDV |
| | | | | AELSISPSDNAVVLTNLLPGTEYVVSVSSVYEQHESTPLRGRQKTGLDS |
| | | | | PTGIDFSDITANSFTVHWIAPRATITGYRIRHHPEHFSGRPREDRVPH |
| | | | | SRNSITLTNLTPGTEYVVSIVALNGREESPLLIGQQSTVSDVPRDLEVV |
| | | | | AATPTSLLISWDAPAVTVRYYRITYGETGGNSPVQEFTVPGSKSTATIS |
| | | | | GLKPGVDYTITVYAVTGRGDSPASSKPISINYRTEIDKPSQMQVTDVQ |
| | | | | DNSISVKWLPSSSPVTGYRVTTTPKNGPGPTKTKTAGPDQTEMTIEG |
| | | | | LQPTVEYVVSVYAQNPSGESQPLVQTAVTNIDRPKGLAFTDVDVDSI |
| | | | | KIAWESPQGQVSRYRVTYSSPEDGIHELFPAPDGEEDTAELQGLRPG |
| | | | | SEYTVSVVALHDDMESQPLIGTQSTAIPAPTDLKFTQVTPTSLSAQW |
| | | | | TPPNVQLTGYRVRVTPKEKTGPMKEINLAPDSSSVVVSGLMVATKYE |
| | | | | VSVYALKDTLTSRPAQGVVTTLENVSPPRRARVTDATETTITISWRTK |
| | | | | TETITGFQVDAVPANGQTPIQRTIKPDVRSYTITGLQPGTDYKIYLYTL |
| | | | | NDNARSSPVVIDASTAIDAPSNLRFLATTPNSLLVSWQPPRARITGYII |
| | | | | KYEKPGSPPREVVPRPRPGVTEATITGLEPGTEYTIYVIALKNNQKSEP |
| | | | | LIGRKKTDELPQLVTLPHPNLHGPEILDVPSTVQKTPFVTHPGYDTGN |
| | | | | GIQLPGTSGQQPSVGQQMIFEEHGFRRTTPPTTATPIRHRPRPYPPN |
| | | | | VGEEIQIGHIPREDVDYHLYPHGPGLNPNASTGQEALSQTTISWAPF |
| | | | | QDTSEYIISCHPVGTDEEPLQFRVPGTSTSATLTGLTRGATYNVIVEAL |
| | | | | KDQQRHKVREEVVTVGNSVNEGLNQPTDDSCFDPYTVSHYAVGDE |
| | | | | WERMSESGFKLLCQCLGFGSGHFRCDSSRWCHDNGVNYKIGEKWD |
| | | | | RQGENGQMMSCTCLGNGKGEFKCDPHEATCYDDGKTYHVGEQW |
| | | | | QKEYLGAICSCTCFGGQRGWRCDNCRRPGGEPSPEGTTGQSYNQYS |
| | | | | QRYHQRTNTNVNCPIECFMPLDVQADREDSRE |
| 49 | HEMO | Hemopexin | P02790 | MARVLGAPVALGLWSLCWSLAIATPLPPTSAHGNVAEGETKPDPDV |
| | | | | TERCSDGWSFDATTLDDNGTMLFFKGEFVWKSHKWDRELISERWK |
| | | | | NFPSPVDAAFRQGHNSVFLIKGDKVWVYPPEKKEKGYPKLLQDEFPG |
| | | | | IPSPLDAAVECHRGECQAEGVLFFQGDREWFWDLATGTMKERSWP |
| | | | | AVGNCSSALRWLGRYYCFQGNQFLRFDPVRGEVPPRYPRDVRDYF |
| | | | | MPCPGRGHGHRNGTGHGNSTHHGPEYMRCSPHLVLSALTSDNHG |
| | | | | ATYAFSGTHYWRLDTSRDGWHSWPIAHQWPQGPSAVDAAFSWEE |
| | | | | KLYLVQGTQVYVFLTKGGYTLVSGYPKRLEKEVGTPHGIILDSVDAAFI |
| | | | | CPGSSRLHIMAGRRLWWLDLKSGAQATWTELPWPHEKVDGALCM |
| | | | | EKSLGPNSCSANGPGLYLIHGPNLYCYSDVEKLNAAKALPQPQNVTSL |
| | | | | LGCTH |
| 50 | HPT | Haptoglobin | P00738 | MSALGAVIALLLWGQLFAVDSGNDVTDIADDGCPKPPEIAHGYVEH |
| | | | | SVRYQCKNYYKLRTEGDGVYTLNDKKQWINKAVGDKLPECEADDGC |
| | | | | PKPPEIAHGYVEHSVRYQCKNYYKLRTEGDGVYTLNNEKQWINKAV |
| | | | | GDKLPECEAVCGKPKNPANPVQRILGGHLDAKGSFPWQAKMVSHH |
| | | | | NLTTGATLINEQWLLTTAKNLFLNHSENATAKDIAPTLTLYVGKKQLV |
| | | | | EIEKVVLHPNYSQVDIGLIKLKQKVSVNERVMPICLPSKDYAEVGRVG |
| | | | | YVSGWGRNANFKFTDHLKYVMLPVADQDQCIRHYEGSTVPEKKTPK |
| | | | | SPVGVQPILNEHTFCAGMSKYQEDTCYGDAGSAFAVHDLEEDTWY |
| | | | | ATGILSFDKSCAVAEYGVYVKVTSIQDWVQKTIAEN |
| 51 | IC1 | Plasma | P05155 | MASRLTLLTLLLLLLAGDRASSNPNATSSSSQDPESLQDRGEGKVATT |
| | | protease C1 | | VISKMLFVEPILEVSSLPTTNSTTNSATKITANTTDEPTTQPTTEPTTQP |
| | | inhibitor | | TIQPTQPTTQLPTDSPTQPTTGSFCPGPVTLCSDLESHSTEAVLGDAL |
| | | | | VDFSLKLYHAFSAMKKVETNMAFSPFSIASLLTQVLLGAGENTKTNLE |
| | | | | SILSYPKDFTCVHQALKGFTTKGVTSVSQIFHSPDLAIRDTFVNASRTL |
| | | | | YSSSPRVLSNNSDANLELINTWVAKNTNNKISRLLDSLPSDTRLVLLNA |
| | | | | IYLSAKWKTTFDPKKTRMEPFHFKNSVIKVPMMNSKKYPVAHFIDQT |
| | | | | LKAKVGQLQLSHNLSLVILVPQNLKHRLEDMEQALSPSVFKAIMEKLE |
| | | | | MSKFQPTLLTLPRIKVTTSQDMLSIMEKLEFFDFSYDLNLCGLTEDPDL |
| | | | | QVSAMQHQTVLELTETGVEAAAASAISVARTLLVFEVQQPFLFVLW |
| | | | | DQQHKFPVFMGRVYDPRA |
| 52 | IGG1 | Immunoglob | P01857 | ASTKGPSVFPLAPSSKSTSGGTAALGCLVKDYFPEPVTVSWNSGALTS |
| | | ulin heavy | | GVHTFPAVLQSSGLYSLSSVVTVPSSSLGTQTYICNVNHKPSNTKVDK |
| | | constant | | KVEPKSCDKTHTCPPCPAPELLGGPSVFLFPPKPKDTLMISRTPEVTCV |
| | | gamma 1 | | VVDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPPSR |
| | | | | DELTKNQVSLTCLVKGFYPSDIAVEWESNGQPENNYKTTPPVLDSDG |
| | | | | SFFLYSKLTVDKSRWQQGNVFSCSVMHEALHNHYTQKSLSLSPELQL |
| | | | | EESCAEAQDGELDGLWTTITIFITLFLLSVCYSATVTFFKVKWIFSSVVD |
| | | | | LKQTIIPDYRNMIGQGA |
| 53 | KLKB1 | Plasma | P03952 | MILFKQATYFISLFATVSCGCLTQLYENAFFRGGDVASMYTPNAQYC |
| | | Kallikrein | | QMRCTFHPRCLLFSFLPASSINDMEKRFGCFLKDSVTGTLPKVHRTG |
| | | | | AVSGHSLKQCGHQISACHRDIYKGVDMRGVNFNVSKVSSVEECQKR |
| | | | | CTNNIRCQFFSYATQTFHKAEYRNNCLLKYSPGGTPTAIKVLSNVESG |
| | | | | FSLKPCALSEIGCHMNIFQHLAFSDVDVARVLTPDAFVCRTICTYHPN |
| | | | | CLFFTFYTNVWKIESQRNVCLLKTSESGTPSSSTPQENTISGYSLLTCKR |
| | | | | TLPEPCHSKIYPGVDFGGEELNVTFVKGVNVCQETCTKMIRCQFFTYS |
| | | | | LLPEDCKEEKCKCFLRLSMDGSPTRIAYGTQGSSGYSLRLCNTGDNSV |
| | | | | CTTKTSTRIVGGTNSSWGEWPWQVSLQVKLTAQRHLCGGSLIGHQ |
| | | | | WVLTAAHCFDGLPLQDVWRIYSGILNLSDITKDTPFSQIKEIIIHQNYK |
| | | | | VSEGNHDIALIKLQAPLNYTEFQKPICLPSKGDTSTIYTNCWVTGWGF |
| | | | | SKEKGEIQNILQKVNIPLVTNEECQKRYQDYKITQRMVCAGYKEGGK |
| | | | | DACKGDSGGPLVCKHNGMWRLVGITSWGEGCARREQPGVYTKVA |
| | | | | EYMDWILEKTQSSDGKAQMQSPA |
| 54 | THRB | Prothrombin | P00734 | MAHVRGLQLPGCLALAALCSLVHSQHVFLAPQQARSLLQRVRRANT |
| | | | | FLEEVRKGNLERECVEETCSYEEAFEALESSTATDVFWAKYTACETAR |
| | | | | TPRDKLAACLEGNCAEGLGTNYRGHVNITRSGIECQLWRSRYPHKPE |
| | | | | INSTTHPGADLQENFCRNPDSSTTGPWCYTTDPTVRRQECSIPVCGQ |
| | | | | DQVTVAMTPRSEGSSVNLSPPLEQCVPDRGQQYQGRLAVTTHGLP |
| | | | | CLAWASAQAKALSKHQDFNSAVQLVENFCRNPDGDEEGVWCYVA |
| | | | | GKPGDFGYCDLNYCEEAVEEETGDGLDEDSDRAIEGRTATSEYQTFF |
| | | | | NPRTFGSGEADCGLRPLFEKKSLEDKTERELLESYIDGRIVEGSDAEIG |
| | | | | MSPWQVMLFRKSPQELLCGASLISDRWVLTAAHCLLYPPWDKNFTE |
| | | | | NDLLVRIGKHSRTRYERNIEKISMLEKIYIHPRYNWRENLDRDIALMKL |
| | | | | KKPVAFSDYIHPVCLPDRETAASLLQAGYKGRVTGWGNLKETWTAN |
| | | | | VGKGQPSVLQVVNLPIVERPVCKDSTRIRITDNMFCAGYKPDEGKRG |
| | | | | DACEGDSGGPFVMKSPFNNRWYQMGIVSWGEGCDRDGKYGFYT |
| | | | | HVFRLKKWIQKVIDQFGE |
| 55 | TRFE | Serotransferr | P02787 | MRLAVGALLVCAVLGLCLAVPDKTVRWCAVSEHEATKCQSFRDHM |
| | | in | | KSVIPSDGPSVACVKKASYLDCIRAIAANEADAVTLDAGLVYDAYLAP |
| | | | | NNLKPVVAEFYGSKEDPQTFYYAVAVVKKDSGFQMNQLRGKKSCHT |
| | | | | GLGRSAGWNIPIGLLYCDLPEPRKPLEKAVANFFSGSCAPCADGTDFP |
| | | | | QLCQLCPGCGCSTLNQYFGYSGAFKCLKDGAGDVAFVKHSTIFENLA |
| | | | | NKADRDQYELLCLDNTRKPVDEYKDCHLAQVPSHTVVARSMGGKE |
| | | | | DLIWELLNQAQEHFGKDKSKEFQLFSSPHGKDLLFKDSAHGFLKVPP |
| | | | | RMDAKMYLGYEYVTAIRNLREGTCPEAPTDECKPVKWCALSHHERL |
| | | | | KCDEWSVNSVGKIECVSAETTEDCIAKIMNGEADAMSLDGGFVYIA |
| | | | | GKCGLVPVLAENYNKSDNCEDTPEAGYFAVAVVKKSASDLTWDNLK |
| | | | | GKKSCHTAVGRTAGWNIPMGLLYNKINHCRFDEFFSEGCAPGSKKD |
| | | | | SSLCKLCMGSGLNLCEPNNKEGYYGYTGAFRCLVEKGDVAFVKHQT |
| | | | | VPQNTGGKNPDPWAKNLNEKDYELLCLDGTRKPVEEYANCHLARAP |
| | | | | NHAVVTRKDKEACVHKILRQQQHLFGSNVTDCSGNFCLFRSETKDLL |
| | | | | FRDDTVCLAKLHDRNTYEKYLGEEYVKAVGNLRKCSTSSLLEACTFRRP |
| 56 | VTNC | Vitronectin | P04004 | MAPLRPLLILALLAWVALADQESCKGRCTEGFNVDKKCQCDELCSYY |
| | | | | QSCCTDYTAECKPQVTRGDVFTMPEDEYTVYDDGEEKNNATVHEQ |
| | | | | VGGPSLTSDLQAQSKGNPEQTPVLKPEEEAPAPEVGASKPEGIDSRP |
| | | | | ETLHPGRPQPPAEEELCSGKPFDAFTDLKNGSLFAFRGQYCYELDEKA |
| | | | | VRPGYPKLIRDVWGIEGPIDAAFTRINCQGKTYLFKGSQYWRFEDGV |
| | | | | LDPDYPRNISDGFDGIPDNVDAALALPAHSYSGRERVYFFKGKQYWE |
| | | | | YQFQHQPSQEECEGSSLSAVFEHFAMMQRDSWEDIFELLFWGRTS |
| | | | | AGTRQPQFISRDWHGVPGQVDAAMAGRIYISGMAPRPSLAKKQRF |
| | | | | RHRNRKGYRSQRGHSRGRNQNSRRPSRATWLSLFSSEESNLGANNY |
| | | | | DDYRMDWLVPATCEPIQSVFFFSGDKYYRVNLRTRRVDTVDPPYPRS |
| | | | | IAQYWLGCPAPGHL |
| 57 | AACT | Alpha-1- | P01011 | MERMLPLLALGLLAAGFCPAVLCHPNSPLDEENLTQENQDRGTHVD |
| | | antichymotry | | LGLASANVDFAFSLYKQLVLKAPDKNVIFSPLSISTALAFLSLGAHNTTL |
| | | psin | | TEILKGLKFNLTETSEAEIHQSFQHLLRTLNQSSDELQLSMGNAMFVK |
| | | | | EQLSLLDRFTEDAKRLYGSEAFATDFQDSAAAKKLINDYVKNGTRGKI |
| | | | | TDLIKDLDSQTMMVLVNYIFFKAKWEMPFDPQDTHQSRFYLSKKKW |
| | | | | VMVPMMSLHHLTIPYFRDEELSCTVVELKYTGNASALFILPDQDKME |
| | | | | EVEAMLLPETLKRWRDSLEFREIGELYLPKFSISRDYNLNDILLQLGIEE |
| | | | | AFTSKADLSGITGARNLAVSQVVHKAVLDVFEEGTEASAATAVKITLL |
| | | | | SALVETRTIVRFNRPFLMIIVPTDTQNIFFMSKVTNPKQA |
| 101 | KNG1 | Kininogen-1 | P01042 | MKLITILFLCSRLLLSLTQESQSEEIDCNDKDLFKAVDAALKKYNSQNQ |
| | | | | SNNQFVLYRITEATKTVGSDTFYSFKYEIKEGDCPVQSGKTWQDCEYK |
| | | | | DAAKAATGECTATVGKRSSTKFSVATQTCQITPAEGPVVTAQYDCLG |
| | | | | CVHPISTQSPDLEPILRHGIQYFNNNTQHSSLFMLNEVKRAQRQVVA |
| | | | | GLNFRITYSIVQTNCSKENFLFLTPDCKSLWNGDTGECTDNAYIDIQL |
| | | | | RIASFSQNCDIYPGKDFVQPPTKICVGCPRDIPTNSPELEETLTHTITKL |
| | | | | NAENNATFYFKIDNVKKARVQVVAGKKYFIDFVARETTCSKESNEELT |
| | | | | ESCETKKLGQSLDCNAEVYVVPWEKKIYPTVNCQPLGMISLMKRPPG |
| | | | | FSPFRSSRIGEIKEETTVSPPHTSMAPAQDEERDSGKEQGHTRRHDW |
| | | | | GHEKQRKHNLGHGHKHERDQGHGHQRGHGLGHGHEQQHGLGH |
| | | | | GHKFKLDDDLEHQGGHVLDHGHKHKHGHGHGKHKNKGKKNGKH |
| | | | | NGWKTEHLASSSEDSTTPSAQTQEKTEGPTPIPSLAKPGVTVTFSDFQ |
| | | | | DSDLIATMMPPISPAPIQSDDDWIPDIQIDPNGLSFNPISDFPDTTSP |
| | | | | KCPGRPWKSVSEINPTTQMKESYYFDLTDGLS |
| 102 | C4BPA | C4b-binding | P04003 | MHPPKTPSGALHRKRKMAAWPFSRLWKVSDPILFQMTLIAALLPAV |
| | | protein alpha | | LGNCGPPPTLSFAAPMDITLTETRFKTGTTLKYTCLPGYVRSHSTQTLT |
| | | chain | | CNSDGEWVYNTFCIYKRCRHPGELRNGQVEIKTDLSFGSQIEFSCSEG |
| | | | | FFLIGSTTSRCEVQDRGVGWSHPLPQCEIVKCKPPPDIRNGRHSGEE |
| | | | | NFYAYGFSVTYSCDPRFSLLGHASISCTVENETIGVWRPSPPTCEKITC |
| | | | | RKPDVSHGEMVSGFGPIYNYKDTIVFKCQKGFVLRGSSVIHCDADSK |
| | | | | WNPSPPACEPNSCINLPDIPHASWETYPRPTKEDVYVVGTVLRYRCH |
| | | | | PGYKPTTDEPTTVICQKNLRWTPYQGCEALCCPEPKLNNGEITQHRK |
| | | | | SRPANHCVYFYGDEISFSCHETSRFSAICQGDGTWSPRTPSCGDICNF |
| | | | | PPKIAHGHYKQSSSYSFFKEEIIYECDKGYILVGQAKLSCSYSHWSAPA |
| | | | | PQCKALCRKPELVNGRLSVDKDQYVEPENVTIQCDSGYGVVGPQSIT |
| | | | | CSGNRTWYPEVPKCEWETPEGCEQVLTGKRLMQCLPNPEDVKMAL |
| | | | | EVYKLSLEIEQLELQRDSARQSTLDKEL |

Table 6A and Table 6B identify and define the N-glycan and O-glycan structures, respectively, that are included in either Table 1, Table 2, or Table 8B as Glycan Structure GL No's. Both Tables 6A and 6B identify a coded representation of the composition for each glycan structure included in either Table 1, Table 2, or Table 8B. As used herein, the 4-digit GL NO. is a designation that represents the number of hexoses, the number of HexNAcs, the number of Fucoses, and the number of Neuraminic Acids.

TABLE 6A
N-Glycan Symbol Structure GL NOS: Composition and Symbol Structure
in accordance with Table 1, Table 2, and Table 8B

| | | Glycan Symbol | Glycan |
|---|---|---|---|
| Glycan | Glycan Composition | Structure | Mass |
| 3410 | Hex(3)HexNAc(4)Fuc(1)NeuAc(0) | 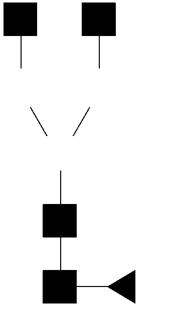 | 1444.533838 |
| 3510 | Hex(3)HexNAc(5)Fuc(1)NeuAc(0) | 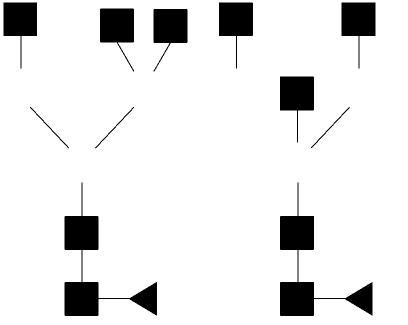 | |
| 5410 | Hex(5)HexNAc(4)Fuc(0)NeuAc(1) | 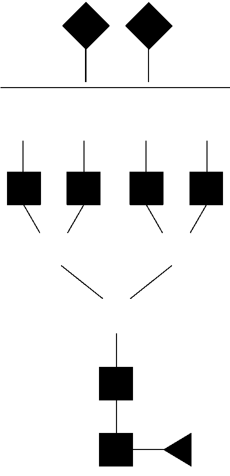 | 1913.676982 |
| 5402 | Hex(5)HexNAc(4)Fuc(0)NeuAc(2) | 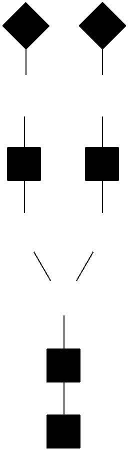 | 2204.77239 |
| 5411 | Hex(5)HexNAc(4)Fuc(1)NeuAc(1) | 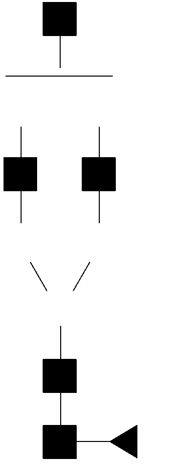 | 2059.73489 |
| 5412 | Hex(5)HexNAc(4)Fuc(1)NeuAc(2) | 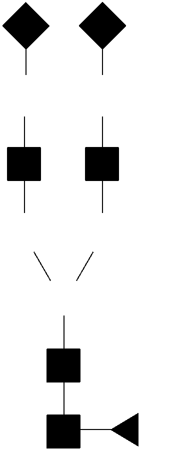 | 2350.830298 |
| 6200 | Hex(6)HexNAc(2)Fuc(0)NeuAc(0) | 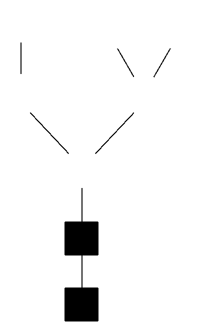 | 1378.475656 |
| 6502 | Hex(6)HexNAc(5)Fuc(0)NeuAc(2) | 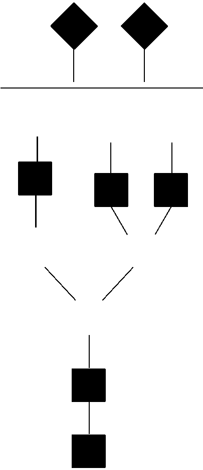 | 2569.90458 |
| 6503 | Hex(6)HexNAc(5)Fuc(0)NeuAc(3) | 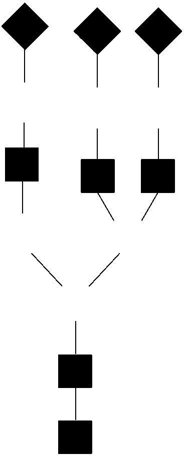 | 2860.99999 |
| 6512 | Hex(6)HexNAc(5)Fuc(1)NeuAc(2) | 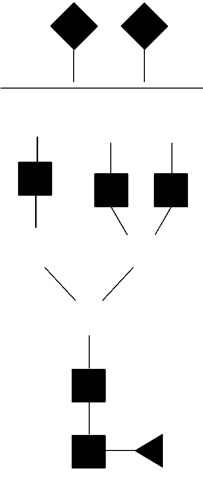 | |
| 6513 | Hex(6)HexNAc(5)Fuc(1)NeuAc(3) | 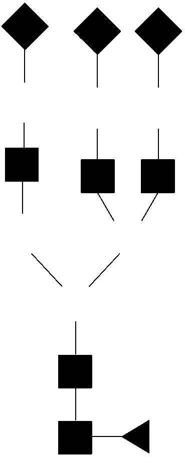 | 3007.057896 |
| 7602 | Hex(7)HexNAc(6)Fuc(0)NeuAc(2) | 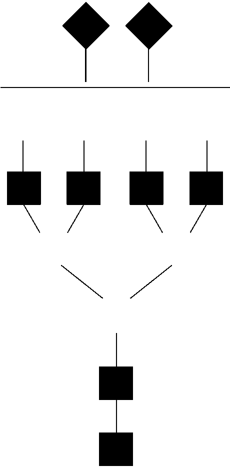 | 2935.03677 |
| 7612 | Hex(7)HexNAc(6)Fuc(1)NeuAc(2) | 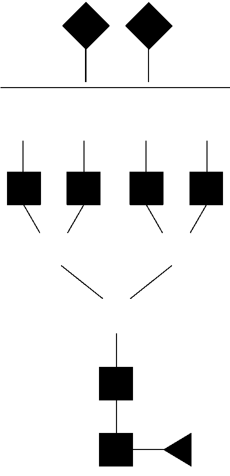 | |
| 7613 | Hex(7)HexNAc(6)Fuc(1)NeuAc(3) | 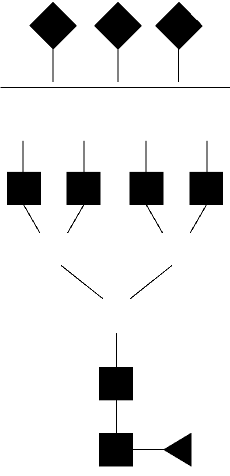 | 3390.20058 |
| 7614 | Hex(7)HexNAc(6)Fuc(1)NeuAc(4) | 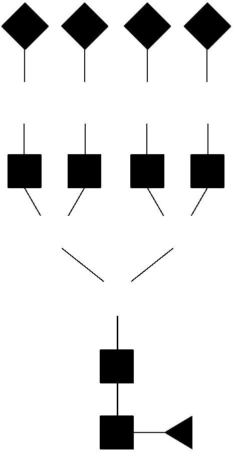 | |
| 7624 | Hex(7)HexNAc(6)Fuc(2)NeuAc(4) | 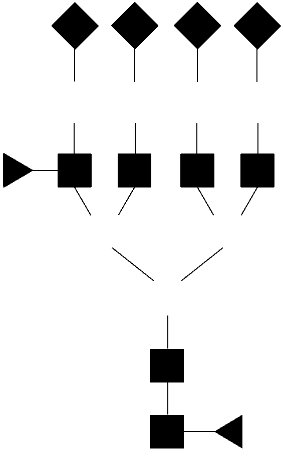 | |

TABLE 6B
O-Glycan GL NOS: Composition and Symbol Structure in accordance
with Table 1, Table 2, and Table 8B.

| Glycan | | Glycan Symbol | |
|---|---|---|---|
| GL No. | Glycan Composition | Structure | Glycan Mass |
| 3310 | Hex(3)HexNAc(3)Fuc(1)NeuAc(0) | 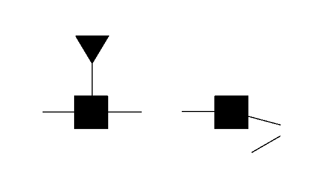 | |

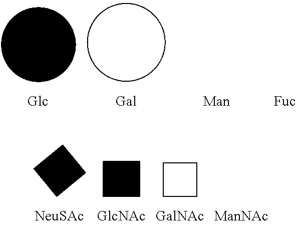

Table 6A and Table 6B illustrate the symbol structure and composition of detected glycan moieties that correspond to the glycopeptides of Table 1, Table 2, or Table 8B based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. It should be noted that the Glycan Structure GL NO. 3310 is an O-linked glycan that is in Table 6B and that N-linked glycans are in Table 6A. For reference, N-linked glycans have a glycan attached to the amino acid asparagine and O-linked glycans have a glycan attached to either a serine or a threonine.

The identity of the various monosaccharides is illustrated by the Legend section located at the end of Table 6B. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these clasess are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Table 6A and Table 6B, for some entries, there are two symbol structures provided for one Glycan Structure GL NO such as, for example, Glycan Structure GL NO 4500. Thus, the identify of a peptide that references a Glycan Structure GL NO that has two symbol structures could be either of the two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an advanced adenoma or CRC disease state. A transition includes a precursor ion and at least one product ion grouping. As reviewed herein, the peptide structures in Table 1, Table 2, or Table 8B as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, advanced adenoma or CRC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 3A or an m/z ratio within an identified m/z ratio as provided in Table 3A. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

VII.A.1 Representative Experimental Results—Subject Sample Model & Corresponding Training of Said Model based on Table 1

Colorectal cancer (CRC) is the second most common cause of cancer related mortality in the US and nearly 150,000 new cases of CRC are diagnosed annually with 50,000 deaths. Approximately 80% of CRCs arise from adenomatous polyps, also called adenomas, which grow slowly and progress to dysplasia and then cancer over a period of approximately ten years. Screening for advanced adenomas (AAs) and CRC is important given the ability to identify premalignant lesions and early-stage malignancy nearly 2-3 years before cases with symptoms. In the US, 1 in 3, or 23 million eligible people are not screened for colon cancer. As a result, 60% of colon cancers are diagnosed at a late stage when the cancer has already metastasized consequently making the treatment process more challenging. Early identification and intervention can lead to better prognosis for a patient and reduce CRC associated morbidity and mortality.

The main screening methods currently for CRC include: colonoscopy, fecal occult blood test (FOBT), fecal immunochemical test (FIT), computed tomography (CT) colonography, flexible sigmoidoscopy, or a multitarget stool DNA test. Carcinoembryonic antigen (CEA) functions as a nonspecific marker of recurrence for CRC but there is an unmet need for the development of blood-based diagnostic tests for advanced adenomas and CRC which provides a quick, cost-effective, non-invasive method for screening and serial monitoring. Current non-invasive tests that are commercially available to patients have lower sensitivities for detecting advanced adenomas.

Glycosylation is the most common post-translational modification in proteins and nearly 50-70% of serum proteins are glycosylated. Protein and lipid linked glycans play pivotal roles in cell differentiation, cell-cell interactions, cell growth and adhesion, and immune response. Aberrant glycosylation is a universal feature in various steps of malignant transformation and tumor progression. However, the glycoproteome has never been interrogated at scale due to the structural diversity and complexity of glycans and the massive amount of information that needs to be processed. A proprietary platform to interrogate the glycoproteome was developed which involves using LC-MS (liquid chromatography and mass spectrometry) coupled with artificial intelligence to determine relative glycopeptide (GP) abundances. Preliminary results using glycoproteins markers in blood samples are promising, demonstrating the ability to differentiate normal tissue from advanced adenoma from colorectal cancer.

In this study, the utility of glycoproteomics as a CRC screening mechanism was evaluated. 3,002 patients were enrolled and data was prospectively collected who underwent colonoscopy. The performance characteristics of a glycoproteomics test in the detection of advanced adenomas and CRC was evaluated.

Eligible participants aged 45-85 years were identified from patients scheduled for a colonoscopy as part of their standard of care. Individuals with any active malignancy were excluded from the study. Subjects consented to provide a blood specimen within a 90 day range prior to their scheduled colonoscopy. A range of clinical data was collected which included: demographics, medical history, lifestyle and behavioral factors, colonoscopy report, and pathology report when applicable.

Additionally, serum specimens from AA and CRC patients were sourced from Indivumed (Hamburg, Germany) and iSpecimen (Lexington, MA). AA and CRC specimens were collected either before or after colonoscopy but before surgery. All specimens had been obtained prior to therapeutic intervention. Histopathological data was available in AA and CRC patients, and clinical stage data in CRC patients. Histopathological analysis of tissue samples yielded information about the benign or malignant nature of tissue samples collected. Conventional adenomas are defined as tubular, tubulovillous or villous adenomas. Serrated lesions are defined as sessile serrated adenomas (SSA), traditional serrated adenomas (TSA), or hyperplastic polyps (HP). AA was defined as a polyp measuring ≥1 cm in the greatest dimension, a polyp of any size with high-grade dysplasia, or a tubulovillous/villous polyp of any size. Tubular adenomas, SSA, and TSA measuring ≥1 cm-1.4 cm, and HP of any size were categorized as low-risk AA. Tubular adenomas or serrated lesions (except HP) with low-grade dysplasia measuring ≥1.5 cm, conventional adenomas or serrated lesions with high-grade dysplasia of any size, and tubulovillous/villous polyps of any size were categorized as high-risk AA.

The primary objective was to utilize glycoproteomic markers in blood samples in subjects undergoing colonoscopy to detect advanced adenomas and CRC.

Blood samples were collected by venipuncture into 8.5 mL serum separator tubes (SST) (Becton-Dickinson, Franklin Lakes, NJ). The tubes were centrifuged at 1,000-1,300 g for 10 minutes after being left to clot. Subsequently, the biospecimen was transferred to Nalgene cryotubes (Thermo Scientific, Waltham, MA) with preprinted unique barcodes and frozen at −80° C. (+/−) 10°. The entire procedure was completed within 2 hours.

A central biorepository received all blood specimens. All specimens were inspected, separated into aliquots, and frozen at −80° C. upon receipt.

Pooled human serum (MilliporeSigma, St. Louis, MO) was used for quality control, assay normalization, and calibration purposes. Dithiothreitol (DTT) and iodoacetamide (IAA) were purchased from MilliporeSigma (St. Louise, MO). LC-MS grade trypsin, acetonitrile, and formic acid were sourced from Thermo Scientific (Waltham, MA), and stable isotope-labeled peptides from Vivitide (Gardner, MA). Serum samples were treated with DTT and IAA to reduce disulfide bonds and alkylate sulfhydryl groups, respectively, followed by digestion with trypsin at 37° C. for 18 hours. The digestion was quenched by adding formic acid to each sample to a final concentration of 1% (v/v) followed by addition of a cocktail of stable isotope-labeled peptide internal standards at known concentrations.

Trypsin-digested serum samples were separated using a Waters (Milford, MA) ACQUITY Peptide HSS T3 column (2.1 mm internal diameter×150 mm length, 1.8 μm particle size) and an Agilent 1290 Infinity UHPLC system. The mobile phase A consisted of 0.1 formic acid in water (v/v), and the mobile phase B of 0.1 formic acid in acetonitrile (v/v), with the flow rate set at 0.5 mL/minute. After UHPLC separation, the peptides and GPs in the serum samples were introduced into an Agilent 6495C triple quadrupole MS through electrospray ionization operated in positive ion mode and quantified in a targeted manner by dynamic multiple reaction monitoring (dMRM). Samples were injected in a randomized fashion as to underlying phenotype, and reference pooled serum digests were injected interspersed with study samples. Laboratory testing was performed without knowledge of the clinical findings of each participant.

MRM analysis was performed sequentially in two separate experiments. The initial analysis measured 607 peptides and GPs derived from 75 high-abundance serum glycoproteins at concentrations of ≥10 μg/ml (the “Full” MRM panel). The MRM transitions comprised 532 GPs and 75 non-modified peptides, one each from the 75 proteins from which the monitored GPs were derived. PeakBoundaryNet, in-house software based on recurrent neural networks for spectrogram feature recognition and integration, was used to integrate chromatogram peaks and to obtain molecular abundance for each analyte.

Normalized abundances of peptides and GPs were assessed in samples from colonoscopy negative controls, and from patients diagnosed with non-AAs, AAs and CRC. Raw abundances were normalized in several ways. First, relative abundance was calculated for all glycopeptides as the ratio of abundance of any given glycopeptide to the abundance of a distinct, non-glycosylated peptide from the same protein. Second, heavy-isotope-labeled internal standards with known peptide concentrations were spiked-in to every sample, engendering an absolute quantification for each of the 75 endogenous proteins. Third, an approximate glycopeptide concentration was derived by multiplying the relative abundance by the absolute protein concentration. Finally, all features were log-normalized prior to univariate or multivariable analysis.

Separately, non-site-specific glycan features were calculated to approximate a purely glycomic approach. In this analysis, glycan fraction was calculated as the ratio of the sum of abundance of every glycopeptide with a given glycan, to the sum of abundances of all glycopeptides monitored in the Full MRM panel.

To compare phenotype groups, age- and sex-adjusted linear regression was employed separately for each biomarker, with clinical phenotype serving as the binary independent variable of interest and normalized biomarker expression as the dependent variable. Fold-changes for individual peptides and GPs were calculated, and p-values were corrected for multiple comparisons using the Benjamini-Hochberg method to calculate false discovery rate (FDR). Differences between phenotype groups were considered statistically significant for markers with FDR under 0.05. Hierarchical clustering was used to visualize the top differential expression results via heatmap.

The full MRM analysis was utilized primarily as a feature selection step, such that a reduced set of peptides and glycopeptides could be chosen for inclusion in an optimized LC-MS assay with reduced run time. Linear regression results from the training set, and markers retained in a preliminary multivariable analysis across training, validation, and test sets, resulted in an optimized assay with 21 peptides and glycopeptides (Table 2).

A total of 3,002 subjects were enrolled in the prospective observational study; [2,304 (76.7%)] subjects were evaluated following first data lock for initial training and validation purposes, the additional [698 (23.3%)] subjects subsequently added as an independent hold-out set. This resulted in a total study population of [2,734] subjects with a median age of [61.3] years and [57%] female: [64%] of subjects were non-Hispanic Caucasians and [53%] are of average risk.

The total number of subjects included in the training, validation and hold-out test sets for the full MRM analysis was 3427, consisting of 537 Subjects with CRC (16%) (231 cases with CRC Stage I (6.7%), 180 cases with CRC Stage II (5.3%), 74 cases with CRC Stage III (2.2%) and 50 cases with CRC Stage IV (1.5%), 448 subjects with AA (293 cases ≥1 cm without villous features or HGD (8.5%); 155 cases with HGD (4.5%), 1301 colonoscopy negative controls (38%) and 1141 subjects with non-AA (33%). Colonoscopy negative controls are samples from a subject who had a recent colonoscopy, and no polyp, lesions, or abnormal tissue (e.g., tumor or cancer cells) was found in the colon. Non-AA are samples from a subject who has an adenoma, but it is not advanced. In various embodiments, a recent colonoscopy can be a situation where the blood sample is taken from the subject within 1 day, 1 week, 4 weeks, 10 weeks, or 6 months of the colonoscopy procedure.

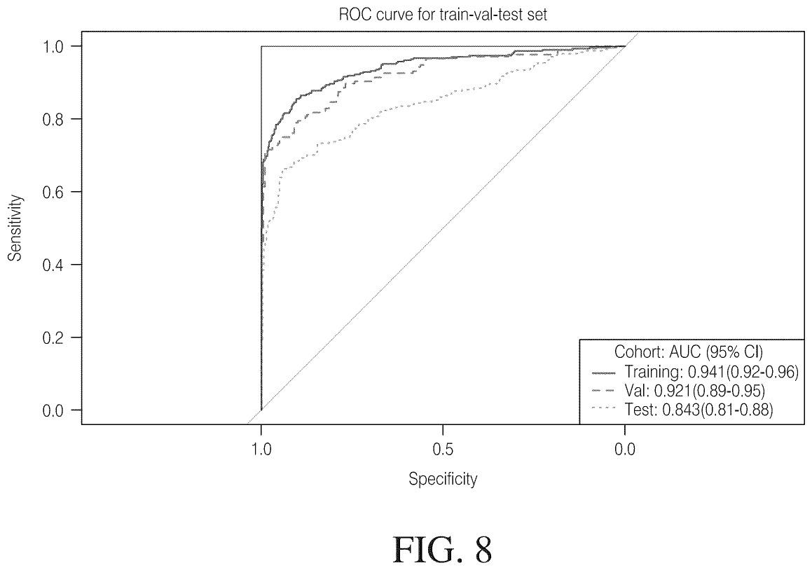

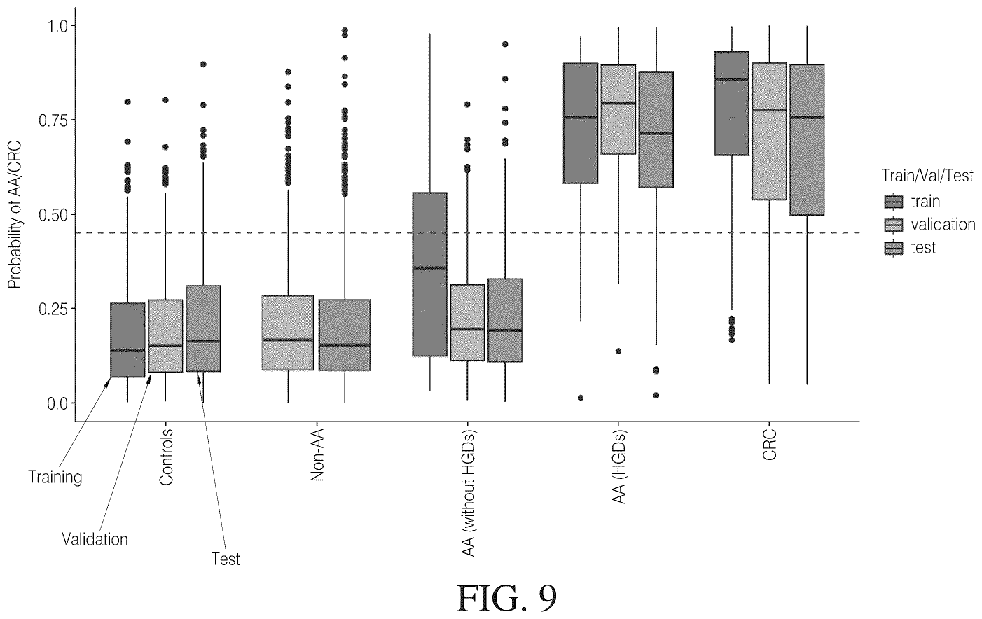

To assess the suitability of serum glycoproteomics as a tool to differentiate AA and CRC samples from negative colonoscopy controls and non-AA control samples, a multivariable elastic net logistic regression model was built with the model input of log-transformed normalized data. Repeated cross-validation in the training set established optimal hyperparameters to balance sensitivity and specificity (cross-validated F1=0.945, alpha=0.5, lambda=0.035) and retained 31 GP markers in this preliminary model (Table 1). Table 1B shows the corresponding coefficients and associated genes for each of the 31 biomarkers of Table 1. The model achieved similar performance in both the validation set (accuracy=0.81, sensitivity=0.60, specificity=0.89) and testing sets (accuracy=0.78, sensitivity=0.53, specificity=0.89). We observed an area under the receiver operating characteristic (AuROC) of 0.94, 0.92 and 0.84 for the training, validation and the test set respectively (FIG. 8). Sensitivity metrics in the hold-out test set across all CRC stages was 79.19% [95% confidence interval (CI): 72.37-84.98] (89.29% for stage I/II [95% CI: 82.03-94.34] and 60.66% for stage III/IV [95% CI: 47.31-72.93]), for high-grade dysplasia AA samples was 85.11% [95% CI: 71.69-93.8] and for AAs without high-grade dysplasias was 14.47% [95% CI: 9.3-21.09], based on choosing a cutoff score of 0.45. The model achieved specificity of 89.96% [95% CI: 85.82-93.23] for colonoscopy negative controls and 88.97% [95% CI: 85.98-91.52] for non-advanced adenoma controls in the hold-out test set. The probability distributions of this classifier were similar across training, validation and test sets (FIG. 9).

For each of the cohorts (control, Non-AA, AA (without HGDs), AA (HGDs), and CRC), the probability of AA/CRC was shown in FIG. 9 for the training, validation, and test data sets. For the control cohort of FIG. 9, the training, validation, and test boxplots are expressly labeled for clarity and are organized in the order from left to right respectively. For the other cohorts of Non-AA, AA (without HGDs), AA (HGDs), and CRC, this convention of illustrating the boxplot format order for the training, validation, and test data, respectively, from left to right was followed. FIG. 9 illustrated that CRC and AA (HGDs) samples can be identified from sample groups that contain controls, Non-AA, and AA (without HGDs).

TABLE 1B
Multivariable classifier model classifying CRC and AA from the healthy samples; consisting
of 31 markers with non-zero coefficients [retain coefficients and gene]

| | | | | | Linking Site | |
|---|---|---|---|---|---|---|
| Pep | | | | | Position in | Glycan |
| SEQ | | | | | Protein | Structure |
| ID | PS-NAME | Coefficients | gene | Uniprot ID | Sequence | GL NO. |
| 1 | A1AT_271_5401 | 0.188535343 | SERPINA1 | P01009 | 271 | 5401 |
| 2 | A1AT_271_5412 | 0.002264864 | SERPINA1 | P01009 | 271 | 5412 |
| 3 | AFAM_33_5402 | 0.141152932 | AFM | P43652 | 33 | 5402 |
| 4 | AGP1_93_7613 | 0.061461567 | ORM1 | P02763 | 93 | 7613 |
| 5 | AGP12_56_6503 | −0.160850869 | ORM1&ORM2 | P02763 | 56 | 6503 |
| | | | | &P19652 | | |
| 6 | ANGT_47_5401 | −0.086604811 | AGT | P01019 | 47 | 5401 |
| 7 | ANT_128_5402 | 0.516185171 | SERPINC1 | P01008 | 128 | 5402 |
| 8 | ANT_187_5412 | 0.17152446 | SERPINC1 | P01008 | 187 | 5412 |
| 9 | APOE_212_NONGLYCOSYLATED | −1.542534618 | APOE | P02649 | 212 | NONGLYCOSYLATED |
| 10 | APOH_253_NONGLYCOSYLATED | −0.275212338 | APOH | P02749 | 253 | NONGLYCOSYLATED |
| 11 | CERU_358_5402 | −2.596563439 | CP | P00450 | 358 | 5402 |
| 12 | CERU_358_NONGLYCOSYLATED | −0.471255011 | CP | P00450 | 358 | NONGLYCOSYLATED |
| 13 | CFAI_70_5401 | −0.303323208 | CFI | P05156 | 70 | 5401 |
| 14 | CO3_85_6200 | −2.389657074 | C3 | P01024 | 85 | 6200 |
| 15 | FETUA_156_6513 | 0.486580572 | AHSG | P02765 | 156 | 6513 |
| 16 | FINC_542_6502 | 0.292738872 | FN1 | P02751 | 542 | 6502 |
| 17 | HEMO_453_5402 | −4.069906362 | HPX | P02790 | 453 | 5402 |
| 18 | HEMO_64_5402 | 2.001197933 | HPX | P02790 | 64 | 5402 |
| 19 | HPT_241_5402 | 1.423383438 | HP | P00738 | 241 | 5402 |
| 20 | HPT_241_6512 | 0.073955443 | HP | P00738 | 241 | 6512 |
| 21 | HPT_184_7602 | −0.117058831 | HP | P00738 | 184 | 7602 |
| 22 | IC1_238_5402 | 2.101680272 | SERPING1 | P05155 | 238 | 5402 |
| 23 | IC1_238_5411 | −0.672632462 | SERPING1 | P05155 | 238 | 5411 |
| 24 | IC1_253_5412 | −0.424075252 | SERPING1 | P05155 | 253 | 5412 |
| 25 | IGG1_297_3310 | 0.156952954 | IGHG1 | P01857 | 297 | 3310 |
| 26 | IGG1_297_3410 | 0.581077912 | IGHG1 | P01857 | 297 | 3410 |
| 27 | KLKB1_396_5401 | 0.222235622 | KLKB1 | P03952 | 396 | 5401 |
| 28 | THRB_416_5402 | 0.312527477 | F2 | P00734 | 416 | 5402 |
| 29 | TRFE_630_5402 | −1.061076767 | TF | P02787 | 630 | 5402 |
| 30 | TRFE_630_5402_NH3LOSS | −0.952948251 | TF | P02787 | 630 | 5402 |
| 31 | VTNC_169_5402 | 2.633288545 | VTN | P04004 | 169 | 5402 |

### VII.A.2 Representative Experimental Results-Subject Sample Model & Corresponding Training of Said Model Based on Table 2

Colorectal cancer (CRC) remains a major cause of global morbidity and mortality despite current detection modalities. During malignant transformation, there is a substantial alteration in the immune response and corresponding protein glycosylation within the colonic crypt. Consequently, abnormal protein glycosylation has emerged as a promising and novel biomarker category. This study evaluates serum glycoproteomic markers for the early CRC detection.

Utilizing a glycoproteomic profiling platform that combines liquid-chromatography/mass-spectrometry and artificial-intelligence-powered data processing, we assessed glycopeptide and non-glycosylated peptide quantification transitions in serum of subjects at risk for CRC. The samples were split into training, validation and hold-out testing sets. Statistical analyses were performed on normalized data from the optimized assay to develop and validate a classifier to predict probability of CRC/AAs against controls.

We analyzed 1,356 prospectively collected samples and 681 biorepository samples [545 (27%) CRC, 383 (19%) AAs, 154 (8%) non-AAs, 955 colonoscopy negative controls (NEG) (47%)]. We identified 84, 89, and 16 GPs/peptides with statistically significant abundance differences (FDR<0.001), when comparing CRCs with NEGs, high-grade dysplastic (HGD) AAs with NEGs, and AAs to non-AAs respectively. A subset of 21 of these biomarkers were used to generate a multivariable classifier model using the training and validation data sets. The 21 biomarkers are shown in Table 2. When the classifier was applied to the test set, it yielded an area under the receiver-operating characteristic (AuROC) of 0.78. Using a defined cutoff, the sensitivity of the classifier for all stages of CRC was 80.9% (85.0% stage 1 and 2), for AAs was 43.8% and for HGDs was 89.6% with specificity of 90.4% for NEGs and 89.6% for non-AAs in the test set.

Once the 31 biomarkers were determined, a development process was performed to improve the measurement of the relevant peptides and glycopeptides. Given the reduced number of MS transitions (corresponding to the 607 to 31 biomarkers), the chromatography column length and the runtime can be reduced for performing the measurements. For example, the HSS T3 column was reduced from a 150 mm length to a 50 mm length. The reduced run time of the optimized assay allows for longer dwell time in the mass spectrometer, and thus improved sensitivity and reproducibility for each transition. The modified method, however, results in differential abundances, thus preliminary (“Full”) models were retrained with normalized abundance values from the optimized assay. To this end, a representative subset of all patient samples were run a second time on the reduced, optimized panel, with the intent of locking a multivariable classifier from the 31 biomarkers available. Abundances and concentrations of those biomarkers were normalized as described above for the Full MRM assay.

For supervised multivariable modeling, the patient pool was randomly split into training, validation, and hold-out test sets, stratifying on the following patient features: age, sex, colonoscopy findings, disease/non-disease indication, and sample source. Colonoscopy negative controls, high-risk AA, and early-stage CRC (stage 1 and 2) samples were split into training, validation and testing sets in a ratio of 50-20-20; low risk AA and non-AA samples were not utilized in training, and were split 50% in validation and 50% in testing set.

To perform binary classification and predict probability of AA/CRC, repeated ten-fold cross-validation loops were used to evaluate elastic net logistic regression models. Elastic net shrinkage parameters were selected to optimize performance in the validation set. The probability cutoff for the classifier was selected from possibilities on the ROC curves to promote balanced sensitivity and specificity metrics in the training and validation data, and the model was locked. Finally, this locked model was applied to the held-out test set to obtain unbiased performance metrics in patients not utilized for any of the aforementioned training steps. The libraries ‘stats’, ‘dplyr’, ‘caret’ and ‘ggplot2’ from the R programming language and the library ‘Scikit-learn’ (https://scikit-learn.org/stable/) for Python were used for all statistical analyses and machine learning models.

The total number of subjects included in the training, validation and hold-out test sets for the optimized assay analysis was 2037, consisting of 545 Subjects with CRC (27%) (230 cases with CRC Stage I (11%), 178 cases with CRC Stage II (8.7%), 82 cases with CRC Stage III (4.0%) and 54 cases with CRC Stage IV (2.7%)), 383 subjects with AA (241 cases ≥1 cm without villous features or HGD (12%); 142 cases with HGD (7.0%)), 955 colonoscopy negative controls (47%) and 154 subjects with non-AA (7.6%).

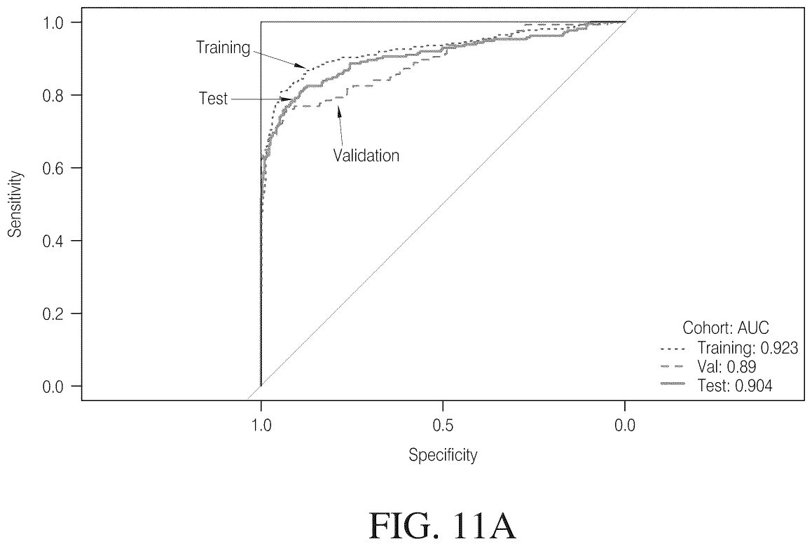

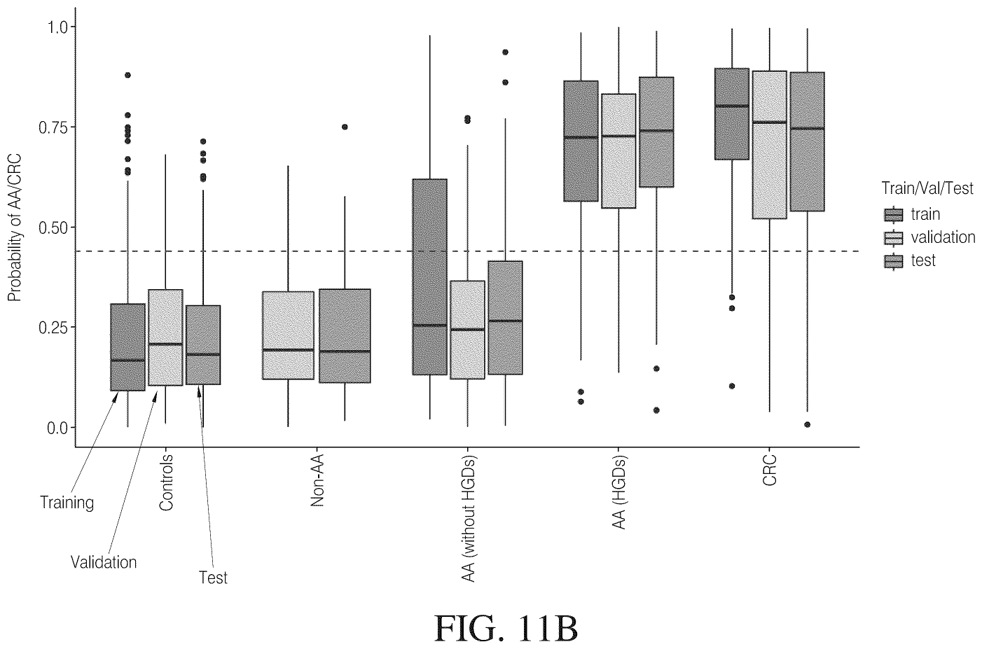

In an embodiment, the model was trained using one group with AA/CRC I/CRC II samples and the other group with Colonoscopy Negative Controls/Non-AA samples. In another embodiment, the model was trained using one group with high-risk AA/CRC I/CRC II samples and the other group with Colonoscopy Negative Controls/Non-AA samples. In another embodiment, the model was trained using one group with high-risk AA/CRC I/CRC II samples and the other group with Colonoscopy Negative Controls. In yet another embodiment, the model was trained using one group with AA/CRC I/CRC II samples and the other group with Colonoscopy Negative Controls. A multivariable elastic net logistic regression model was built using log-normalized data features using the optimized assay method to differentiate AA/CRC samples from the colonoscopy negative controls and non-AA control samples; that retained features related to 21 GP markers (Table 2). Repeated cross-validation in the training set achieved optimal hyperparameters (cross-validated F1=0.92, alpha=0.5, lambda=0.05) and observed an area under the receiver operating characteristic (AuROC) of 0.92, 0.89 and 0.90 for the training, validation and the test set respectively (FIG. 11A). Sensitivity metrics in the hold-out test set across all CRC stages was 80.9% [95% CI: 74.75-86.12] (84.96% for stage I/II [95% CI: 77.74-90.57] and 72.73% for stage III/IV [95% CI: 60.36-82.97]), for high-grade dysplasia AA samples was 89.58% [95% CI: 77.34-96.53] and for AAs without high-grade dysplasias was 22.86% [95% CI: 15.23-32.07], based on choosing a cutoff score of 0.44. The model achieved specificity of 90.35% [95% CI: 86.52-93.4] for colonoscopy negative controls and 89.61% [95% CI: 80.55-95.41] for non-advanced adenoma controls in the hold-out test set (Table 2B). The probability distributions of this classifier were similar across training, validation and hold-out test sets (FIG. 11B).

Referring back to FIG. 11B, each of the cohorts (Control, Non-AA, AA (without HGDs), AA (HGDs), and CRC), the probability of having the CRC/AA disease statewas shown in FIG. 11B for the training, validation, and test data sets. For each of the cohorts Control, Non-AA, AA (without HGDs), AA (HGDs), and CRC, the convention of illustrating the boxplot format order for the training, validation, and test data, respectively, is from left to right. For the Control cohort of FIG. 11B, the training, validation, and test boxplots are expressly labeled for clarity and are organized in the order from left to right respectively. FIG. 11B illustrates that CRC and AA (HGDs) samples can be identified from samples that are from the Controls, Non-AA, and AA (without HGDs). The control samples can be from healthy subjects who do not have CRC or an adenoma as confirmed with a colonoscopy. The non-AA samples contain inflammatory polyps of any size, polypoid colon mucosa of any size, tubular adenoma <10 mm, sessile serrated adenoma <10 mm, traditional serrated adenoma <10 mm, and hyperplastic polyps <10 mm.

One or more features (e.g., relative abundance, concentration, site occupancy) of these peptide structures may be used in the supervised machine learning model described above to generate a disease indicator that predicts the probability of CRC/AA in the validation or test set. In an embodiment, relative abundance can be used for SEQ ID NO: 3-9, 12, 14-16, 18, 25-28, and 31-35 and concentration can also be used for SEQ ID NO: 9, 25, and 26 in the supervised machine learning model to generate a disease indicator that predicts the probability of CRC I-IV/High-Risk AA (HGDs). For example, it should be noted that the same peptide structure (e.g., SEQ ID NO: 9) can be used with a relative abundance and a concentration value for use in the model (e.g., PS-ID 9 of Table 2 and Table 7).

TABLE 2B
Sensitivity and Specificity of the model for detecting AA/CRC
vs. colonoscopy negative controls in the hold-out test set.

| | | Model Performance | |
|---|---|---|---|
| | Colonoscopy | Positive | |
| | (N = 740) | Results | Sensitivity |
| Colonoscopy Finding | no. | no. | % (0.95% CI) |
| CRC (any stage) | 199 | 161 | 80.9 (74.75-86.12) |
| CRC I/II | 133 | 113 | 84.96 (77.74-90.57) |
| CRC and high-grade | 247 | 204 | 82.59 (77.28-87.11) |
| dysplasia adenomas | | | |
| Advanced adenomas, | 48 | 43 | 89.58 (77.34-96.53) |
| high-grade dysplasia | | | |
| Advanced adenomas, | 105 | 24 | 22.86 (15.23-32.07) |
| without high-grade | | | |
| dysplasia | | | |
| | | | Specificity |
| Negative results | 311 | 30 | 90.35 (86.52-93.4) |
| on colonoscopy | | | |
| All Non-advanced | 77 | 8 | 89.61 (80.55-95.41) |
| Adenomas | | | |

Using the biomarkers of Table 2, a model was developed that had biomarker coefficients as shown in Table 7 based on either the relative abundance values or concentration values measured for the biomarkers. The performance metrics of this model were shown in FIG. 11A and FIG. 11B.

TABLE 7
Coefficients for each marker used in a model for
classifying healthy control/non-AA vs CRC/AA

| PS-ID | Pep SEQ | | | |
|---|---|---|---|---|
| No. | ID NO | PS-NAME | Nomalization | Coefficients |
| 32 | 32 | AACT_106_7624 | relative abundance | 0.35852598 |
| 3 | 3 | AFAM_33_5402 | relative abundance | 0.7560825 |
| 4 | 4 | AGP1_93_7613 | relative abundance | 0.65471713 |
| 5 | 5 | AGP12_56_6503 | relative abundance | −0.1415651 |
| 6 | 6 | ANGT_47_5401 | concentration | −0.161707 |
| 7 | 7 | ANT_128_5402 | relative abundance | 2.64150942 |
| 8 | 8 | ANT_187_5412 | relative abundance | 0.35519995 |
| 9 | 9 | APOE_212_NONGLYCOSYLATED | relative abundance | 0.70732409 |
| 9 | 9 | APOE_212_NONGLYCOSYLATED | concentration | −0.1736152 |
| 12 | 12 | CERU_358_NONGLYCOSYLATED | relative abundance | −1.4188542 |
| 33 | 33 | CERU_397_5402 | concentration | −0.2545308 |
| 14 | 14 | CO3_85_6200 | relative abundance | −3.8546522 |
| 15 | 15 | FETUA_156_6513 | relative abundance | 1.8404767 |
| 16 | 16 | FINC_542_6502 | relative abundance | 5.61062556 |
| 18 | 18 | HEMO_64_5402 | relative abundance | 1.89901803 |
| 34 | 34 | HPT_241_6513 | relative abundance | 1.51541522 |
| 25 | 25 | IGG1_297_3310 | concentration | −0.2728438 |
| 25 | 25 | IGG1_297_3310 | relative abundance | 0.03583184 |
| 26 | 26 | IGG1_297_3410 | concentration | −0.1504665 |
| 26 | 26 | IGG1_297_3410 | relative abundance | 0.40379578 |
| 35 | 35 | IGG1_297_3510 | relative abundance | 0.5620174 |
| 27 | 27 | KLKB1_396_5401 | relative abundance | 1.69359812 |
| 28 | 28 | THRB_416_5402 | relative abundance | 0.69302872 |
| 31 | 31 | VTNC_169_5402 | relative abundance | 1.31767002 |

In various embodiments, using the values of Table 7, a probability can be determined by summing together the product of the concentration (or relative abundance) of each biomarker in the sample and the respective coefficient and then adding the summation and the intercept to yield the logit of a probability score. For example, the logit of the probability, to which the inverse logit function can be applied, is equal to:

$${\sum }_{i = 1}^{i = 35} [ ( Concentration or Relative {Abundance}_{SEQ ID NO : i} ) \times ( {Coefficient}_{SEQ ID NO : i} ) ] + Intercept$$

where i=PS-ID NO,

Under certain circumstances, the PS-ID NO can be used such as 3-9, 12, 14-16, 18, 25-28, and 31-35 using the corresponding relative abundance values and PS-ID NO can be used again such as 9, 25, and 26 using the corresponding concentration values.

The peptide structure data can comprise normalized concentration data, wherein the normalized concentration data is a function of at least one of peptide abundance data, corresponding internal standard abundance data, a spike-in concentration value, and a dilution factor. The peptide structure profile for a given peptide structure may include a corresponding feature—relative abundance, concentration, site occupancy—for that peptide structure. The relative abundance of a glycopeptide can be calculated by dividing the abundance of the glycopeptide by the abundance of a quantification peptide. The quantification peptide can be an abundance of a peptide structure that is similar or the same as the peptide portion of the relevant glycopeptide. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance (e.g., PS-ID NO: 9) for a corresponding peptide structure and a second peptide structure profile may include a concentration (e.g., PS-ID NO: 9) for the same corresponding peptide structure (e.g., SEQ ID NO: 9).

TABLE 3B
Mass Spectrometry-Related Characteristics for the Peptide Structures
associated with CRC/AA in accordance with Table 2

| | 1st | 1st | 2nd | 2nd | 1^{st} | 2nd | | Collision |
|---|---|---|---|---|---|---|---|---|
| PS-ID | Precursor | Precursor | Precursor | Precursor | Product | Product | RT - | Energy - |
| No. | m/z | charge | m/z | charge | m/z | m/z | min | V |
| 32 | 1243.3 | 5 | N/A | N/A | 274.1 | N/A | 38.3 | 35 |
| 3 | 851.1 | 4 | 1134.5 | 3 | 366.1 | 1398.6 | 11.4 | 20 |
| 4 | 1323.1 | 4 | N/A | N/A | 366.1 | N/A | 23.1 | 33 |
| 5 | 1220.1 | 3 | N/A | N/A | 274.1 | 999.4 | 5.4 | 35 |
| 6 | 891 | 5 | N/A | N/A | 366.1 | 913.5 | 23.9 | 15 |
| 7 | 1019.2 | 4 | N/A | N/A | 366.1 | N/A | 40.8 | 20 |
| 8 | 1133.5 | 4 | N/A | N/A | 366.1 | N/A | 40.9 | 23 |
| 9 | 749.8 | 2 | N/A | N/A | 827.4 | 642.4 | 19.1 | 22 |
| 12 | 820.9 | 2 | N/A | N/A | 1052.5 | 678.3 | 31.5 | 25 |
| 33 | 1084.2 | 4 | N/A | N/A | 204.1 | N/A | 27.4 | 35 |
| 14 | 1212.2 | 3 | 1211.9 | 3 | 1230.1 | 366.1 | 27.2 | 35 |
| 15 | 1196 | 4 | N/A | N/A | 366.1 | 274.1 | 27.6 | 30 |
| 16 | 1127.2 | 4 | N/A | N/A | 366.1 | N/A | 14.2 | 30 |
| 18 | 1184.5 | 4 | N/A | N/A | 204.1 | N/A | 40.5 | 35 |
| 34 | 1201.5 | 4 | N/A | N/A | 366.1 | N/A | 30.9 | 30 |
| 25 | 1216.5 | 2 | N/A | N/A | 204.1 | N/A | 7.9 | 35 |
| 26 | 879 | 3 | N/A | N/A | 204.1 | 1392.6 | 7.9 | 21 |
| 35 | 946.5 | 3 | N/A | N/A | 204.1 | 1392.6 | 8.1 | 15 |
| 27 | 1069.2 | 4 | N/A | N/A | 204.1 | N/A | 39.8 | 25 |
| 28 | 1143.1 | 3 | N/A | N/A | 274.1 | 712.4 | 23.71 | 25 |
| 31 | 1039.4 | 3 | N/A | N/A | 366.1 | 1114.6 | 24.8 | 32 |

Table 3B shows various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a first precursor charge for the peptide or glycopeptide. Similarly, the second precursor m/z represents a ratio value associated with an ionized form having a second precursor charge for the peptide or glycopeptide. The first precursor ion is associated with a first product ion having a m/z ratio that was formed from a collision and the second precursor ion is associated with a second product ion having a m/z ratio that was formed from a collision. Under certain circumstances, the first precursor and the second precursor may be the same, but the associated first and second product m/z ratios are different.

TABLE 4C
Peptide SEQ ID NOS in accordance with Table 2

| | | Prot. | Pept. |
|---|---|---|---|
| PS-ID | | SEQ | SEQ |
| No. | Peptide Sequence | ID NO | ID NO |
| 32 | FNLTETSEAEIHQSFQHLLR | 57 | 32 |
| | (SEQ ID NO: 32) | | |
| 3 | DIENFNSTQK | 37 | 3 |
| | (SEQ ID NO: 3) | | |
| 4 | QDQCIYNTTYLNVQR | 38 | 4 |
| | (SEQ ID NO: 4) | | |
| 5 | NEEYNK | 38 or 39 | 5 |
| | (SEQ ID NO: 5) | | |
| 6 | VYIHPFHLVIHNESTCEQLAK | 40 | 6 |
| | (SEQ ID NO: 6) | | |
| 7 | LGACNDTLQQLMEVFK | 41 | 7 |
| | (SEQ ID NO: 7) | | |
| 8 | SLTFNETYQDISELVYGAK | 41 | 8 |
| | (SEQ ID NO: 8) | | |
| 9 | AATVGSLAGQPLQER | 42 | 9 |
| | (SEQ ID NO: 9) | | |
| 12 | AGLQAFFQVQECNK | 44 | 12 |
| | (SEQ ID NO: 12) | | |
| 33 | ENLTAPGSDSAVFFEQGTTR | 44 | 33 |
| | (SEQ ID NO: 33) | | |
| 14 | TVLTPATNHMGNVTFTIPANR | 46 | 14 |
| | (SEQ ID NO: 14) | | |
| 15 | VCQDCPLLAPLNDTR | 47 | 15 |
| | (SEQ ID NO: 15) | | |
| 16 | HEEGHMLNCTCFGQGR | 48 | 16 |
| | (SEQ ID NO: 16) | | |
| 18 | CSDGWSFDATTLDDNGTMLFFK | 49 | 18 |
| | (SEQ ID NO: 18) | | |
| 34 | VVLHPNYSQVDIGLIK | 50 | 34 |
| | (SEQ ID NO: 34) | | |
| 25 | EEQYNSTYR | 52 | 25 |
| | (SEQ ID NO: 25) | | |
| 26 | EEQYNSTYR | 52 | 26 |
| | (SEQ ID NO: 26) | | |
| 35 | EEQYNSTYR | 52 | 35 |
| | (SEQ ID NO: 35) | | |
| 27 | IVGGTNSSWGEWPWQVSLQVK | 53 | 27 |
| | (SEQ ID NO: 27) | | |
| 28 | NFTENDLLVR | 54 | 28 |
| | (SEQ ID NO: 28) | | |
| 31 | NGSLFAFR | 56 | 31 |
| | (SEQ ID NO: 31) | | |

TABLE 4D
Markers and Protein Positions in accordance with Table 2

| PS- | | | Start | End |
|---|---|---|---|---|
| ID No. | PS-NAME | Peptide Sequence | Position | Position |
| 32 | AACT_106_7624 | FNLTETSEAEIHQSFQHLLR | 105 | 124 |
| | | (SEQ ID NO: 32) | | |
| 3 | AFAM_33_5402 | DIENFNSTQK | 28 | 37 |
| | | (SEQ ID NO: 3) | | |
| 4 | AGP1_93_7613 | QDQCIYNTTYLNVQR | 87 | 101 |
| | | (SEQ ID NO: 4) | | |
| 5 | AGP12_56_6503 | NEEYNK | 52 | 57 |
| | | (SEQ ID NO: 5) | | |
| 6 | ANGT_47_5401 | VYIHPFHLVIHNESTCEQLAK | 36 | 56 |
| | | (SEQ ID NO: 6) | | |
| 7 | ANT_128_5402 | LGACNDTLQQLMEVFK | 124 | 139 |
| | | (SEQ ID NO: 7) | | |
| 8 | ANT_187_5412 | SLTFNETYQDISELVYGAK | 183 | 201 |
| | | (SEQ ID NO: 8) | | |
| 9 | APOE_212_NONGLY- | AATVGSLAGQPLQER | 210 | 224 |
| | COSYLATED | (SEQ ID NO: 9) | | |
| 12 | CERU_358_NONGLY- | AGLQAFFQVQECNK | 346 | 359 |
| | COSYLATED | (SEQ ID NO: 12) | | |
| 33 | CERU_397_5402 | ENLTAPGSDSAVFFEQGTTR | 396 | 415 |
| | | (SEQ ID NO: 33) | | |
| 14 | CO3_85_6200 | TVLTPATNHMGNVTFTIPANR | 74 | 94 |
| | | (SEQ ID NO: 14) | | |
| 15 | FETUA_156_6513 | VCQDCPLLAPLNDTR | 145 | 159 |
| | | (SEQ ID NO: 15) | | |
| 16 | FINC_542_6502 | HEEGHMLNCTCFGQGR | 535 | 550 |
| | | (SEQ ID NO: 16) | | |
| 18 | HEMO_64_5402 | CSDGWSFDATTLDDNGTMLFFK | 50 | 71 |
| | | (SEQ ID NO: 18) | | |
| 34 | HPT_241_6513 | VVLHPNYSQVDIGLIK | 236 | 251 |
| | | (SEQ ID NO: 34) | | |
| 25 | IGG1_297_3310 | EEQYNSTYR | 176 | 184 |
| | | (SEQ ID NO: 25) | | |
| 26 | IGG1_297_3410 | EEQYNSTYR | 176 | 184 |
| | | (SEQ ID NO: 26) | | |
| 35 | IGG1_297_3510 | EEQYNSTYR | 176 | 184 |
| | | (SEQ ID NO: 35) | | |
| 27 | KLKB1_396_5401 | IVGGTNSSWGEWPWQVSLQVK | 391 | 411 |
| | | (SEQ ID NO: 27) | | |
| 28 | THRB_416_5402 | NFTENDLLVR | 416 | 425 |
| | | (SEQ ID NO: 28) | | |
| 31 | VTNC_169_5402 | NGSLFAFR | 169 | 176 |
| | | (SEQ ID NO: 31) | | |

VII.A.3 Representative Experimental Results—Glycosylation Pattern in Transition from Healthy to CRC Based on Table 8B

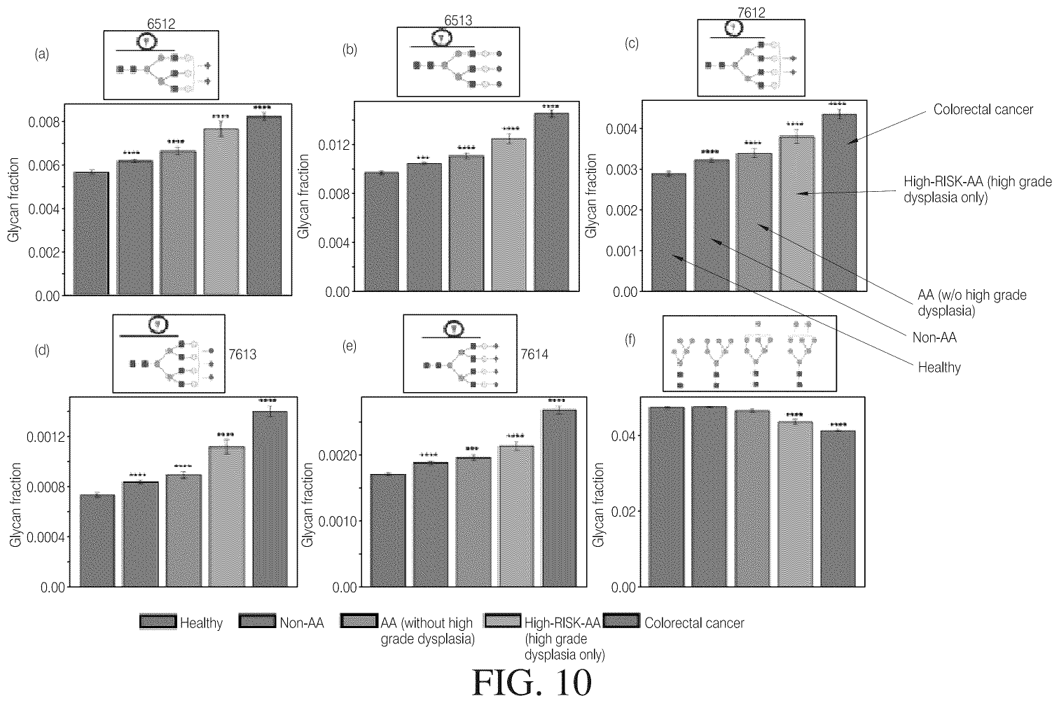

Changing glycosylation patterns were noted during the progression from healthy to AA to CRC by analyzing the glycans across all the glycopeptides included in the panel (FIG. 10(a) to FIG. 10(f)). For N-glycopeptides, the tri- and tetra-antennary fucosylated species were found to steadily increase in the progression from healthy to AA to CRC. The high mannose fraction of glycopeptides was significantly reduced in patients with high-risk AA (high grade dysplasia only) and subjects with CRC, and it appeared to decrease in AA (without high grade dysplasia) subjects compared with healthy subjects.

FIG. 10(a) to FIG. 10(e) are bar graph representations of the glycan fraction for fucosylated tri-antennary glycans with (a) two sialic acids and (b) with three sialic acid; fucosylated tetra-antennary glycans with (c) two sialic acids, (d) three sialic acids, and (e) four sialic acids. It should be noted that FIG. 10(a) to FIG. 10(e) represent glycopeptides having glycans with the Glycan GL NOs: 6512, 6513, 7612, 7613, and 7614, respectively. FIG. 10(f) is a bar graph representation of the glycan fraction for high mannose (M5-M9) glycans containing glycopeptides (*p-value<=0.05, **p-value<=0.01, ***p-value<=0.001, **** p-value<=0.0001). FIG. 10(a) to FIG. 10(e) indicate that the glycan fraction of the tri- and tetra-antennary glycopeptides increased and correlated with the progression of the CRC disease state. The glycan fraction of the tri- and tetra-antennary glycopeptides were relatively low for the healthy cohort (i.e., leftmost bar for each of the cohorts shown in FIG. 10(a) to FIG. 10(e)). For each of the bar graph representations (FIG. 10(a) to FIG. 10(e)), the cohorts are listed in the order from left to right starting with healthy, Non-AA, AA (without high grade dysplasia), High-RISK-AA (with high grade dysplasia only), and colorectal cancer. As an example, the cohort groups for FIG. 10(c) are expressly labeled for clarity. The cohort groups for FIG. 10(a) to FIG. 10(b), and FIG. 10(d) to FIG. 10(f) are organized in the same manner as FIG. 10(c).

Referring back to FIG. 10(a) to FIG. 1(e), the amount or proportion of glycopeptides having fucosylated glycans progressively increased from the following cohorts healthy, Non-AA, AA (without high grade dysplasia), High-RISK-AA (high grade dysplasia), and colorectal cancer. Thus, higher amounts of glycopeptides having fucosylated glycans can be correlated for identifying High-RISK-AA (high grade dysplasia) and colorectal cancer. In addition, higher amounts of glycopeptides having tri-antennary glycans, tetra-antennary glycans, alone or in combination can be correlated for identifying High-RISK-AA (high grade dysplasia) and colorectal cancer. A subject that transitions from an adenoma to CRC state can have significant changes in fucosylated glycan motifs of as shown in FIG. 10(a) to FIG. 10(e).

In various embodiments, the amount or proportion of glycopeptides having fucosylated glycans with either tri- and tetra-antennary glycan structure progressively increased from the following cohorts healthy, Non-AA, AA (without high grade dysplasia), High-RISK-AA (high grade dysplasia), and colorectal cancer. In various embodiments, fucosylated glycopeptides having tri-antennary glycan structure can be a glycopeptide including a N-linked glycan having the Glycan GL NO: 6512 and/or 6523. In various embodiments, fucosylated glycopeptides having tetra-antennary glycan structure can be a glycopeptide including a N-linked glycan having the Glycan GL NO: 7612, 7613, and/or 7614. The glycopeptides represented by FIG. 10(a) to FIG. 10(f) are shown in Table 8A.

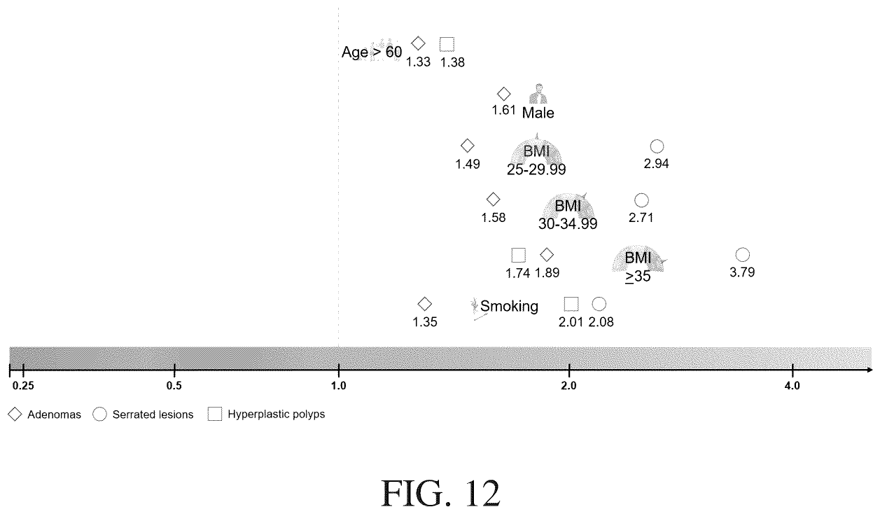

TABLE 8A
Glycopeptide structures represented in FIG. 10(a) to FIG. 10(f) that were used for calculating the glycan fractions.

| 6512 - FIG. 12(a) | 6513 - FIG. 12(b) | 7612 - FIG. 12(c) | 7613- FIG. 12(d) | 7614- FIG. 12(e) | high mannose- FIG. |
|---|---|---|---|---|---|
| A1AT_107_6512 | A1AT_107_6513 | AGP1_103_7612 | AGP1_93_7613 | AACT_106_7614 | A2MG_247_5200 |
| AACT_271_6512 | AACT_106_6513 | AGP1_93_7612 | AGP12_72_7613 | AGP1_103_7614 | A2MG_247MC_5200 |
| CERU_762_6512 | AACT_271_6513 | | AGP12_72MC_7613 | AGP1_93_7614 | A2MG_869_5200 |
| FETUA_176_6512 | AGP1_103_6513 | | HPT_241_7613 | AGP12_72_7614 | A2MG_869_6200 |
| HPT_241_6512 | AGP1_93_6513 | | | AGP12_72MC_7614 | CO2_621_5200 |
| KNG1_205_6512 | AGP12_56_6513 | | | AGP2_103_7614 | CO2_621_6200 |
| | AGP12_72_6513 | | | | CO2_621_7200 |
| | AGP12_72MC_6513 | | | | CO2_621_8200 |
| | AGP2_103_6513 | | | | CO3_85_5200 |
| | APOH_162_6513 | | | | CO3_85_6200 |
| | C4BPA_221_6513 | | | | CO3_85_7200 |
| | CERU_138_6513 | | | | IGM_439_5200 |
| | CERU_397_6513 | | | | IGM_439_6200 |
| | CERU_762_6513 | | | | IGM_439_6200_Z4 |
| | FETUA_156_6513 | | | | IGM_439_7200 |
| | FETUA_176_6513 | | | | IGM_439_8200 |
| | HEMO_187_6513 | | | | IGM_439_9200 |
| | HPT_184_6513 | | | | |
| | HPT_241_6513 | | | | |
| | IC1_253_6513 | | | | |
| | KNG1_169_6513 | | | | |
| | KNG1_205_6513 | | | | |
| | KNG1_294_6513 | | | | |
| | TRFE_630_6513 | | | | |
| | VTNC_242_6513 | | | | |

TABLE 8B
Set of Fucosylated Glycopeptide Structures Associated with CRC that include tri-antennary and
tetra-antennary glycan structures in accordance with FIG. 10(a) to FIG. 10(e) and Table 8A.

| | | | Prot | Pep | Glycos site | Glyco site | Glycan | Mono- |
|---|---|---|---|---|---|---|---|---|
| PS-ID | | | SEQ ID | SEQ ID | within | within | Struct | isotopic |
| No. | PS-NAME | Protein Name | NO | NO | Prot SEQ | Pept SEQ | GL No. | mass |
| 36 | A1AT_107_6512 | Alpha-1- | 36 | 58 | 107 | 14 | 6512 | 6406.77897 |
| | | antitrypsin | | | | | | |
| 37 | AACT_271_6512 | Alpha-1- | 57 | 59 | 271 | 4 | 6512 | 4467.835462 |
| | | antichymotrypsin | | | | | | |
| 38 | CERU_762_6512 | Ceruloplasmin | 44 | 60 | 762 | 9 | 6512 | 4736.95909 |
| 39 | FETUA_176_6512 | Alpha-2-HS- | 47 | 61 | 176 | 11 | 6512 | 5080.108264 |
| | | glycoprotein | | | | | | |
| 40 | HPT_241_6512 | Haptoglobin | 50 | 62 | 241 | 6 | 6512 | 4509.966416 |
| 41 | KNG1_205_6512 | Kininogen-1 | 101 | 63 | 205 | 9 | 6512 | 4128.659418 |
| 42 | A1AT_107_6513 | Alpha-1- | 36 | 64 | 107 | 14 | 6513 | 6697.87438 |
| | | antitrypsin | | | | | | |
| 43 | AACT_106_6513 | Alpha-1- | 57 | 65 | 106 | 2 | 6513 | 5406.244812 |
| | | antichymotrypsin | | | | | | |
| 44 | AACT_271_6513 | Alpha-1- | 57 | 66 | 271 | 4 | 6513 | 4758.930872 |
| | | antichymotrypsin | | | | | | |
| 45 | AGP1_103_6513 | Alpha-1-acid | 38 | 67 | 103 | 2 | 6513 | 3782.440306 |
| | | glycoprotein 1 | | | | | | |
| 46 | AGP1_93_6513 | Alpha-1-acid | 38 | 68 | 93 | 7 | 6513 | 4921.94726 |
| | | glycoprotein 1 | | | | | | |
| 47 | AGP12_56_6513 | Alpha-1-acid | 38 or 39 | 69 | 56 | 5 | 6513 | 3802.397774 |
| | | glycoprotein 1or2 | | | | | | |
| 48 | AGP12_72_6513 | Alpha-1-acid | 38 or 39 | 70 | 72 | 15 | 6513 | 4926.00437 |
| | | glycoprotein 1or2 | | | | | | |
| 49 | AGP12_72MC_6513 | Alpha-1-acid | 38 or 39 | 71 | 72 | 15 | 6513 | 5901.506894 |
| | | glycoprotein 1or2 | | | | | | |
| 50 | AGP2_103_6513 | Alpha-1-acid | 39 | 72 | 103 | 2 | 6513 | 3768.424656 |
| | | glycoprotein 2 | | | | | | |
| 51 | APOH_162_6513 | Beta-2- | 43 | 73 | 162 | 8 | 6513 | 4474.804888 |
| | | glycoprotein1 | | | | | | |
| 52 | C4BPA_221_6513 | C4b-binding | 102 | 74 | 221 | 15 | 6513 | 6378.685664 |
| | | protein alpha chain | | | | | | |
| 53 | CERU_138_6513 | Ceruloplasmin | 44 | 75 | 138 | 10 | 6513 | 4898.89152 |
| 54 | CERU_397_6513 | Ceruloplasmin | 44 | 76 | 397 | 2 | 6513 | 5133.049472 |
| 55 | CERU_762_6513 | Ceruloplasmin | 44 | 77 | 762 | 9 | 6513 | 5028.0545 |
| 56 | FETUA_156_6513 | Alpha-2-HS- | 47 | 78 | 156 | 12 | 6513 | 4777.897142 |
| | | glycoprotein | | | | | | |
| 57 | FETUA_176_6513 | Alpha-2-HS- | 47 | 79 | 176 | 11 | 6513 | 5371.203674 |
| | | glycoprotein | | | | | | |
| 58 | HEMO_187_6513 | Hemopexin | 49 | 80 | 187 | 7 | 6513 | 4410.719444 |
| 59 | HPT_184_6513 | Haptoglobin | 50 | 81 | 184 | 6 | 6513 | 5685.442854 |
| 60 | HPT_241_6513 | Haptoglobin | 50 | 82 | 241 | 6 | 6513 | 4801.061826 |
| 61 | IC1_253_6513 | Plasma protease C1 | 51 | 83 | 253 | 4 | 6513 | 5107.14298 |
| | | inhibitor | | | | | | |
| 62 | KNG1_169_6513 | Kininogen-1 | 101 | 84 | 169 | 8 | 6513 | 5627.30709 |
| 63 | KNG1_205_6513 | Kininogen-1 | 101 | 85 | 205 | 9 | 6513 | 4419.754828 |
| 64 | KNG1_294_6513 | Kininogen-1 | 101 | 86 | 294 | 6 | 6513 | 4437.740894 |
| 65 | TRFE_630_6513 | Serotransferrin | 55 | 87 | 630 | 9 | 6513 | 5521.174696 |
| 66 | VTNC_242_6513 | Vitronectin | 56 | 88 | 242 | 1 | 6513 | 5778.372916 |
| 67 | AGP1_103_7612 | Alpha-1-acid | 38 | 89 | 103 | 2 | 7612 | 3856.477084 |
| | | glycoprotein 1 | | | | | | |
| 68 | AGP1_93_7612 | Alpha-1-acid | 38 | 90 | 93 | 7 | 7612 | 4995.984038 |
| | | glycoprotein 1 | | | | | | |
| 69 | AGP1_93_7613 | Alpha-1-acid | 38 | 91 | 93 | 7 | 7613 | 5287.079448 |
| | | glycoprotein 1 | | | | | | |
| 70 | AGP12_72_7613 | Alpha-1-acid | 38 or 39 | 92 | 72 | 15 | 7613 | 5291.136558 |
| | | glycoprotein 1or2 | | | | | | |
| 71 | AGP12_72MC_7613 | Alpha-1-acid | 38 or 39 | 93 | 72 | 15 | 7613 | 6266.639082 |
| | | glycoprotein 1or2 | | | | | | |
| 72 | HPT_241_7613 | Haptoglobin | 50 | 94 | 241 | 6 | 7613 | 5166.194014 |
| 73 | AACT_106_7614 | Alpha-1- | 57 | 95 | 106 | 2 | 7614 | 6062.47241 |
| | | antichymotrypsin | | | | | | |
| 74 | AGP1_103_7614 | Alpha-1-acid | 38 | 96 | 103 | 2 | 7614 | 4438.667904 |
| | | glycoprotein 1 | | | | | | |
| 75 | AGP1_93_7614 | Alpha-1-acid | 38 | 97 | 93 | 7 | 7614 | 5578.174858 |
| | | glycoprotein 1 | | | | | | |
| 76 | AGP12_72_7614 | Alpha-1-acid | 38 or 39 | 98 | 72 | 15 | 7614 | 5582.231968 |
| | | glycoprotein 1or2 | | | | | | |
| 77 | AGP12_72MC_7614 | Alpha-1-acid | 38 or 39 | 99 | 72 | 15 | 7614 | 6557.734492 |
| | | glycoprotein 1or2 | | | | | | |
| 78 | AGP2_103_7614 | Alpha-1-acid | 39 | 100 | 103 | 2 | 7614 | 4424.652254 |
| | | glycoprotein 2 | | | | | | |

TABLE 3C
Mass Spectrometry-Related Characteristics for the 43 Peptide
Structures associated with CRC in accordance with Table 8B

| | 1st | 1st | 2nd | 2nd | 1^{st} | 2nd | | Collision |
|---|---|---|---|---|---|---|---|---|
| PS-ID | Precursor | Precursor | Precursor | Precursor | Product | Product | RT - | Energy - |
| No. | m/z | charge | m/z | charge | m/z | m/z | min | V |
| 36 | 1282.9 | 5 | N/A | N/A | 366.1 | 1299 | 42.7 | 30 |
| 37 | 1118.2 | 4 | N/A | N/A | 366.1 | 978.5 | 30.6 | 30 |
| 38 | 1186 | 4 | N/A | N/A | 366.1 | 1113 | 19.7 | 36 |
| 39 | 1271 | 4 | N/A | N/A | 366.1 | N/A | 30.1 | 32 |
| 40 | 1128.8 | 4 | N/A | N/A | 366.1 | N/A | 30 | 28 |
| 41 | 1033.9 | 4 | N/A | N/A | 366.1 | 808.9 | 16.5 | 32 |
| 42 | 1341 | 5 | N/A | N/A | 366.1 | 1299 | 43.3 | 34 |
| 43 | 1082.6 | 5 | N/A | N/A | 274.1 | N/A | 37.8 | 30 |
| 44 | 1191.2 | 4 | N/A | N/A | 366.1 | 978.5 | 31.3 | 30 |
| 45 | 1262 | 3 | N/A | N/A | 366.1 | 979.5 | 5.7 | 32 |
| 46 | 1231.8 | 4 | N/A | N/A | 274.1 | N/A | 23.3 | 31 |
| 47 | 1268.8 | 3 | N/A | N/A | 274.1 | 999.4 | 5.3 | 33 |
| 48 | 1233 | 4 | N/A | N/A | 366.1 | 1062.5 | 37.6 | 30 |
| 49 | 1181.9 | 5 | N/A | N/A | 366.1 | 1550.3 | 41 | 29 |
| 50 | 1257.5 | 3 | N/A | N/A | 366.1 | 965.5 | 4.6 | 33 |
| 51 | 1120 | 4 | N/A | N/A | 204.1 | 836.4 | 13 | 35 |
| 52 | 1277.3 | 5 | 1064.6 | 6 | 366.1 | 366.1 | 37.7 | 33 |
| 53 | 1226.2 | 4 | N/A | N/A | 366.1 | 1048.5 | 17.1 | 30 |
| 54 | 1284.8 | 4 | N/A | N/A | 204.1 | N/A | 27.5 | 35 |
| 55 | 1258.5 | 4 | N/A | N/A | 274.1 | 1113 | 20.8 | 30 |
| 56 | 1196 | 4 | N/A | N/A | 366.1 | 274.1 | 27.6 | 30 |
| 57 | 1343.8 | 4 | N/A | N/A | 366.1 | N/A | 30.4 | 34 |
| 58 | 1104.2 | 4 | N/A | N/A | 274.1 | N/A | 21.5 | 25 |
| 59 | 1138.4 | 5 | N/A | N/A | 366.1 | 1441.7 | 34.5 | 28 |
| 60 | 1201.5 | 4 | N/A | N/A | 366.1 | N/A | 30.9 | 30 |
| 61 | 1278.3 | 4 | N/A | N/A | 204.1 | 1152.6 | 35.7 | 40 |
| 62 | 1408.6 | 4 | N/A | N/A | 274.1 | N/A | 31.5 | 35 |
| 63 | 1106.4 | 4 | N/A | N/A | 274.1 | N/A | 16.9 | 25 |
| 64 | 1110.9 | 4 | N/A | N/A | 274.1 | N/A | 22.9 | 25 |
| 65 | 1105.6 | 5 | N/A | N/A | 366.1 | 1359.6 | 33.2 | 27 |
| 66 | 1157.1 | 5 | N/A | N/A | 274.1 | N/A | 37.7 | 30 |
| 67 | 1286.6 | 3 | N/A | N/A | 366.1 | N/A | 5.3 | 32 |
| 68 | 1250.3 | 4 | N/A | N/A | 366.1 | N/A | 22.6 | 31 |
| 69 | 1323.1 | 4 | N/A | N/A | 366.1 | N/A | 23.1 | 33 |
| 70 | 1324.3 | 4 | N/A | N/A | 366.1 | N/A | 37.4 | 25 |
| 71 | 1568.4 | 4 | 1254.9 | 5 | 366.1 | 366.1 | 40.9 | 30 |
| 72 | 1292.8 | 4 | N/A | N/A | 366.1 | 999.5 | 30.7 | 32 |
| 73 | 1214.1 | 5 | N/A | N/A | 274.1 | N/A | 38.3 | 35 |
| 74 | 1110.8 | 4 | N/A | N/A | 366.1 | 979.5 | 5.9 | 27 |
| 75 | 1116.9 | 5 | N/A | N/A | 366.1 | 1060 | 23.8 | 25 |
| 76 | 1397.1 | 4 | N/A | N/A | 1062.5 | N/A | 37.8 | 30 |
| 77 | 1313.1 | 5 | N/A | N/A | 366.1 | 1550.3 | 41.2 | 27 |
| 78 | 1107.4 | 4 | N/A | N/A | 366.1 | 965.5 | 4.7 | 30 |

TABLE 4E
Peptide SEQ ID NOS in accordance with Table 8B

| | | Prot. | Pept. |
|---|---|---|---|
| PS-ID | | SEQ | SEQ |
| No. | Peptide Sequence | ID NO | ID NO |
| 36 | ADTHDEILEGLNFNLTEIPEAQIHEGFQEL | 36 | 58 |
| | LR | | |
| | (SEQ ID NO: 58) | | |
| 37 | YTGNASALFILPDQDK | 57 | 59 |
| | (SEQ ID NO: 59) | | |
| 38 | ELHHLQEQNVSNAFLDK | 44 | 60 |
| | (SEQ ID NO: 60) | | |
| 39 | AALAAFNAQNNGSNFQLEEISR | 47 | 61 |
| | (SEQ ID NO: 61) | | |
| 40 | VVLHPNYSQVDIGLIK | 50 | 62 |
| | (SEQ ID NO: 62) | | |
| 41 | ITYSIVQTNCSK | 101 | 63 |
| | (SEQ ID NO: 63) | | |
| 42 | ADTHDEILEGLNFNLTEIPEAQIHEGFQEL | 36 | 64 |
| | LR | | |
| | (SEQ ID NO: 64) | | |
| 43 | FNLTETSEAEIHQSFQHLLR | 57 | 65 |
| | (SEQ ID NO: 65) | | |
| 44 | YTGNASALFILPDQDK | 57 | 66 |
| | (SEQ ID NO: 66) | | |
| 45 | ENGTISR | 38 | 67 |
| | (SEQ ID NO: 67) | | |
| 46 | QDQCIYNTTYLNVQR | 38 | 68 |
| | (SEQ ID NO: 68) | | |
| 47 | NEEYNK | 38 or 39 | 69 |
| | (SEQ ID NO: 69) | | |
| 48 | SVQEIQATFFYFTPNK | 38 or 39 | 70 |
| | (SEQ ID NO: 70) | | |
| 49 | SVQEIQATFFYFTPNKTEDTIFLR | 38 or 39 | 71 |
| | (SEQ ID NO: 71) | | |
| 50 | ENGTVSR | 39 | 72 |
| | (SEQ ID NO: 72) | | |
| 51 | VYKPSAGNNSLYR | 43 | 73 |
| | (SEQ ID NO: 73) | | |
| 52 | FSLLGHASISCTVENETIGVWRPSPPTCEK | 102 | 74 |
| | (SEQ ID NO: 74) | | |
| 53 | EHEGAIYPDNTTDFQR | 44 | 75 |
| | (SEQ ID NO: 75) | | |
| 54 | ENLTAPGSDSAVFFEQGTTR | 44 | 76 |
| | (SEQ ID NO: 76) | | |
| 55 | ELHHLQEQNVSNAFLDK | 44 | 77 |
| | (SEQ ID NO: 77) | | |
| 56 | VCQDCPLLAPLNDTR | 47 | 78 |
| | (SEQ ID NO: 78) | | |
| 57 | AALAAFNAQNNGSNFQLEEISR | 47 | 79 |
| | (SEQ ID NO: 79) | | |
| 58 | SWPAVGNCSSALR | 49 | 80 |
| | (SEQ ID NO: 80) | | |
| 59 | MVSHHNLTTGATLINEQWLLTTAK | 50 | 81 |
| | (SEQ ID NO: 81) | | |
| 60 | VVLHPNYSQVDIGLIK | 50 | 82 |
| | (SEQ ID NO: 82) | | |
| 61 | VLSNNSDANLELINTWVAK | 51 | 83 |
| | (SEQ ID NO: 83) | | |
| 62 | HGIQYFNNNTQHSSLFMLNEVK | 101 | 84 |
| | (SEQ ID NO: 84) | | |
| 63 | ITYSIVQTNCSK | 101 | 85 |
| | (SEQ ID NO: 85) | | |
| 64 | LNAENNATFYFK | 101 | 86 |
| | (SEQ ID NO: 86) | | |
| 65 | QQQHLFGSNVTDCSGNFCLFR | 55 | 87 |
| | (SEQ ID NO: 87) | | |
| 66 | NISDGFDGIPDNVDAALALPAHSYSGR | 56 | 88 |
| | (SEQ ID NO: 88) | | |
| 67 | ENGTISR | 38 | 89 |
| | (SEQ ID NO: 89) | | |
| 68 | QDQCIYNTTYLNVQR | 38 | 90 |
| | (SEQ ID NO: 90) | | |
| 69 | QDQCIYNTTYLNVQR | 38 | 91 |
| | (SEQ ID NO: 91) | | |
| 70 | SVQEIQATFFYFTPNK | 38 or 39 | 92 |
| | (SEQ ID NO: 92) | | |
| 71 | SVQEIQATFFYFTPNKTEDTIFLR | 38 or 39 | 93 |
| | (SEQ ID NO: 93) | | |
| 72 | VVLHPNYSQVDIGLIK | 50 | 94 |
| | (SEQ ID NO: 94) | | |
| 73 | FNLTETSEAEIHQSFQHLLR | 57 | 95 |
| | (SEQ ID NO: 95) | | |
| 74 | ENGTISR | 38 | 96 |
| | (SEQ ID NO: 96) | | |
| 75 | QDQCIYNTTYLNVQR | 38 | 97 |
| | (SEQ ID NO: 97) | | |
| 76 | SVQEIQATFFYFTPNK | 38 or 39 | 98 |
| | (SEQ ID NO: 98) | | |
| 77 | SVQEIQATFFYFTPNKTEDTIFLR | 38 or 39 | 99 |
| | (SEQ ID NO: 99) | | |
| 78 | ENGTVSR | 39 | 100 |
| | (SEQ ID NO: 100) | | |

TABLE F
Markers and Protein Positions in accordance with Table 8B

| PS- | | | Start | End |
|---|---|---|---|---|
| ID No. | PS-NAME | Peptide Sequence | Position | Position |
| 36 | A1AT_107_6512 | ADTHDEILEGLNFNLTEIPEAQIHEGFQELLR | 94 | 125 |
| | | (SEQ ID NO: 58) | | |
| 37 | AACT_271_6512 | YTGNASALFILPDQDK | 268 | 283 |
| | | (SEQ ID NO: 59) | | |
| 38 | CERU_762_6512 | ELHHLQEQNVSNAFLDK | 754 | 770 |
| | | (SEQ ID NO: 60) | | |
| 39 | FETUA_176_6512 | AALAAFNAQNNGSNFQLEEISR | 166 | 187 |
| | | (SEQ ID NO: 61) | | |
| 40 | HPT_241_6512 | VVLHPNYSQVDIGLIK | 236 | 251 |
| | | (SEQ ID NO: 62) | | |
| 41 | KNG1_205_6512 | ITYSIVQTNCSK | 197 | 208 |
| | | (SEQ ID NO: 63) | | |
| 42 | A1AT_107_6513 | ADTHDEILEGLNFNLTEIPEAQIHEGFQELLR | 94 | 125 |
| | | (SEQ ID NO: 64) | | |
| 43 | AACT_106_6513 | FNLTETSEAEIHQSFQHLLR | 105 | 124 |
| | | (SEQ ID NO: 65) | | |
| 44 | AACT_271_6513 | YTGNASALFILPDQDK | 268 | 283 |
| | | (SEQ ID NO: 66) | | |
| 45 | AGP1_103_6513 | ENGTISR | 102 | 108 |
| | | (SEQ ID NO: 67) | | |
| 46 | AGP1_93_6513 | QDQCIYNTTYLNVQR | 87 | 101 |
| | | (SEQ ID NO: 68) | | |
| 47 | AGP12_56_6513 | NEEYNK | 52 | 57 |
| | | (SEQ ID NO: 69) | | |
| 48 | AGP12_72_6513 | SVQEIQATFFYFTPNK | 58 | 73 |
| | | (SEQ ID NO: 70) | | |
| 49 | AGP12_72MC_6513 | SVQEIQATFFYFTPNKTEDTIFLR | 58 | 81 |
| | | (SEQ ID NO: 71) | | |
| 50 | AGP2_103_6513 | ENGTVSR | 102 | 108 |
| | | (SEQ ID NO: 72) | | |
| 51 | APOH_162_6513 | VYKPSAGNNSLYR | 155 | 167 |
| | | (SEQ ID NO: 73) | | |
| 52 | C4BPA_221_6513 | FSLLGHASISCTVENETIGVWRPSPPTCEK | 207 | 236 |
| | | (SEQ ID NO: 74) | | |
| 53 | CERU_138_6513 | EHEGAIYPDNTTDFQR | 129 | 144 |
| | | (SEQ ID NO: 75) | | |
| 54 | CERU_397_6513 | ENLTAPGSDSAVFFEQGTTR | 396 | 415 |
| | | (SEQ ID NO: 76) | | |
| 55 | CERU_762_6513 | ELHHLQEQNVSNAFLDK | 754 | 770 |
| | | (SEQ ID NO: 77) | | |
| 56 | FETUA_156_6513 | VCQDCPLLAPLNDTR | 145 | 159 |
| | | (SEQ ID NO: 78) | | |
| 57 | FETUA_176_6513 | AALAAFNAQNNGSNFQLEEISR | 166 | 187 |
| | | (SEQ ID NO: 79) | | |
| 58 | HEMO_187_6513 | SWPAVGNCSSALR | 181 | 193 |
| | | (SEQ ID NO: 80) | | |
| 59 | HPT_184_6513 | MVSHHNLTTGATLINEQWLLTTAK | 179 | 202 |
| | | (SEQ ID NO: 81) | | |
| 60 | HPT_241_6513 | VVLHPNYSQVDIGLIK | 236 | 251 |
| | | (SEQ ID NO: 82) | | |
| 61 | IC1_253_6513 | VLSNNSDANLELINTWVAK | 250 | 268 |
| | | (SEQ ID NO: 83) | | |
| 62 | KNG1_169_6513 | HGIQYFNNNTQHSSLFMLNEVK | 162 | 183 |
| | | (SEQ ID NO: 84) | | |
| 63 | KNG1_205_6513 | ITYSIVQTNCSK | 197 | 208 |
| | | (SEQ ID NO: 85) | | |
| 64 | KNG1_294_6513 | LNAENNATFYFK | 289 | 300 |
| | | (SEQ ID NO: 86) | | |
| 65 | TRFE_630_6513 | QQQHLFGSNVTDCSGNFCLFR | 622 | 642 |
| | | (SEQ ID NO: 87) | | |
| 66 | VTNC_242_6513 | NISDGFDGIPDNVDAALALPAHSYSGR | 242 | 268 |
| | | (SEQ ID NO: 88) | | |
| 67 | AGP1_103_7612 | ENGTISR | 102 | 108 |
| | | (SEQ ID NO: 89) | | |
| 68 | AGP1_93_7612 | QDQCIYNTTYLNVQR | 87 | 101 |
| | | (SEQ ID NO: 90) | | |
| 69 | AGP1_93_7613 | QDQCIYNTTYLNVQR | 87 | 101 |
| | | (SEQ ID NO: 91) | | |
| 70 | AGP12_72_7613 | SVQEIQATFFYFTPNK | 58 | 73 |
| | | (SEQ ID NO: 92) | | |
| 71 | AGP12_72MC_7613 | SVQEIQATFFYFTPNKTEDTIFLR | 58 | 81 |
| | | (SEQ ID NO: 93) | | |
| 72 | HPT_241_7613 | VVLHPNYSQVDIGLIK | 236 | 251 |
| | | (SEQ ID NO: 94) | | |
| 73 | AACT_106_7614 | FNLTETSEAEIHQSFQHLLR | 105 | 124 |
| | | (SEQ ID NO: 95) | | |
| 74 | AGP1_103_7614 | ENGTISR | 102 | 108 |
| | | (SEQ ID NO: 96) | | |
| 75 | AGP1_93_7614 | QDQCIYNTTYLNVQR | 87 | 101 |
| | | (SEQ ID NO: 97) | | |
| 76 | AGP12_72_7614 | SVQEIQATFFYFTPNK | 58 | 73 |
| | | (SEQ ID NO: 98) | | |
| 77 | AGP12_72MC_7614 | SVQEIQATFFYFTPNKTEDTIFLR | 58 | 81 |
| | | (SEQ ID NO: 99) | | |
| 78 | AGP2_103_7614 | ENGTVSR | 102 | 108 |
| | | (SEQ ID NO: 100) | | |

Table 1, Table 1B, Table 2, and Table 8B include the Peptide Structure Identification Number (PS-ID No.), Peptide Structure Name (PS-Name), Protein Name, Protein Sequence ID Number (Prot SEQ ID No.), Peptide Sequence ID Number (Pep SEQ ID No.), Glycosylation Site within Protein Sequence (Glyco Site within Prot SEQ), Glycosylation Site within Peptide Sequence (Glyco Site within Pept SEQ), Glycan Structure GL Number (Glycan Struct GL No.), and Monoisotopic Mass. The PS-ID is a reference number for a particular peptide or glycopeptide. The PS Name is a reference code for a peptide or glycopeptide. For example, the glycopeptide IC1_253_5412 (e.g., SEQ ID NO 24) has a prefix portion to indicate that the peptide originated from a protein named IC1, followed by the glycan linking site position in the protein (e.g., the number 253 that is preceded by an underscore and represents a sequential amino acid position in protein IC1), and followed by the glycan structure GL number (e.g., the number 5412 that is preceded by an underscore and represents a glycan composition Hex(5)HexNAc(4)Fuc(1)NeuAc(2)). The PS-Name contains a prefix that represents an abbreviation (that may include a combination of letters and numbers) for a protein abbreviation that corresponds to the Protein Abbreviation of Table 5. The term Glyco Site within Prot SEQ is a number that refers to the sequential position of an amino acid of the corresponding protein in which a glycan is attached. For the Glyco Site within Prot SEQ, the amino acid position of the peptide sequence is defined by the sequentially numbered order of amino acids based on the Uniprot ID of the corresponding protein for the peptide sequence. The term Glyco Site within Pept SEQ is a number that refers to the sequential position of an amino acid of the corresponding peptide in which a glycan is attached. For the Glyco Site within Pept SEQ, the amino acid position of the peptide sequence is defined by the sequentially numbered order of amino acids for the peptide sequence that corresponds to Table 4A to Table 4F. The term Glycan Structure GL No. is a number that corresponds to a symbol structure and a composition of the glycan as indicated in Table 6A and Table 6B. The term monoisotopic mass represents the mass of the glycopeptide in grams per mole.

In some embodiments, the term AGP12 (e.g, SEQ ID No. 5) represents that the glycopeptide is a fragment of either of the proteins AGP1 or AGP2.

In Table 1, Table 2, and Table 8B, if the first number subsequent to the first underscore in the PS-NAME is inconsistent with the Glyco site within Prot SEQ number, then the Glyco site within Prot SEQ number should be used for identification of the peptide. If the second number subsequent to the second underscore in the Peptide Structure (PS) NAME is inconsistent with the Glycan Structure GL NO column number, then the Glycan Structure GL NO column number should be used for identification of the glycan portion of the glycopeptide. If the PS-NAME does not contain any numbers, then the peptide is non-glycosylated. In some instances of the PS-NAME, subsequent to the prefix, there is a number noted with the notation MC that indicates that there was a missed cleavage at position in the peptide sequence as noted by the number.

VII.A.6 Beyond Lifestyle Factors: A Closer Look at Polyp Associations with the Circulating Glycoproteomic Host Response—Table 9

The role of lifestyle and behavior in the development of colon adenomas is still not fully understood. However, it has been observed that abnormal glycosylation of circulating proteins is linked to the neoplastic transformation within colonic crypts. To explore this further, this research utilized advanced liquid chromatography-mass spectrometry (LC-MS) technology enhanced with artificial intelligence (AI) to analyze glycoproteomic profiles. The objective was to examine how lifestyle choices and behavior can influence these profiles and their association with different types of polyps.

Using a platform combining LC-MS and AI-powered data processing to identify glycoprotein biomarkers in peripheral blood, we utilized samples from a large observational study (NCT05445570) to identify participants who had homogenous polyp types: adenomatous (AP), hyperplastic (HP) or serrated (SP) polyps. We used generalized multi-nominal response logit models to examine the relationship between polyp types and lifestyle factors. Lifestyle and concomitant factors included in analysis were age, sex, body mass index, smoking history, alcohol consumption, red meat intake, fruit intake, physical exercise, and aspirin use. Thirty-one biomarkers (see Table 1), previously found to be associated with advanced adenomas and CRC, were interjected as exposures in the model. Each marker is assessed independently for inclusion into our final model. In the final model, we include all lifestyle factors along with the biomarkers found to have a significant association with the presence of AP, SP, or HPs.

In total, 2,044 participants with distinct polyp histologies were enrolled. FIG. 12 demonstrates a significant increase in relative risk for the presence of polyps in patients who were: age >60 years (AP relative risk (RR) 1.33 [CI: 1.07-1.64], HP RR 1.38 [1.01-1.88]); male gender (AP RR 1.61 [1.31-1.98]); or had a BMI≥25. Notably, a BMI≥35 increased relative risk for the presence of a polyp across all polyp types (SP RR 3.79 [1.64-8.69]; AP RR 1.89 [1.36-2.60]; and HP RR 1.74 [1.07-2.81]). From the a priori panel of glycoproteomic markers, 15 markers were found to be associated with an increased risk of presence of a polyp (Table 9); 6 biomarkers demonstrated a significant association while adjusting for lifestyle factors (Table 10). In an embodiment, the 6 glycoprotein biomarkers of Table 10 can correspond to 6 glycopeptide biomarkers having SEQ ID NOS: 4, 12, 13, 16, 24, and 27. Two biomarkers (CERU and KLKB1) were found to have a significant association for the risk of HPs in patients who had a personal history of polyp(s).

TABLE 9
List of the fifteen glycoprotein biomarkers that correspond to a glycopeptide
associated with presence of adenomas, serrated, or hyperplastic polyps.

| SEQ | | | | | Relative |
|---|---|---|---|---|---|
| ID | PS-Name | | | Role in colonic | Risk |
| NO | (Glycopeptide) | Glycoprotein | Function | polyps | (95% CI) |
| 3 | AFAM_33_5402 | Afamin | Vitamin | Downregulated in | Adenoma |
| | | | E-binding | CRC compared to | 3.4 (1.4-8.5) |
| | | | protein | adenomas | |
| 4 | AGP1_93_7613 | Alpha-1-acid | Regulates | Specific glycoforms | Adenoma |
| | | glycoprotein | inflammation- | may be involved in | 1.4 (1.2-1.6) |
| | | (AGP) 1 | related events | polyps/CRC | Hyperplastic |
| | | | | | 1.3 (1.0-1.6) |
| 5 | AGP12_56_6503 | AGP1 or AGP2 | See above for | See above for AGP1 | Adenoma |
| | | | AGP1 AGP2: | AGP2: Positively | 0.6 (0.4-0.8) |
| | | | Drug binding | associated with | |
| | | | and immuno- | CRC development | |
| | | | modulating | | |
| 6 | ANGT_47_5401 | Angiotensinogen | Precursor of | Increased | Adenoma |
| | | | angiotensin | production can lead | 0.7 (0.6-0.9) |
| | | | peptides | to CRC | |
| | | | | angiogenesis | |
| 8 | ANT_187_5412 | Antithrombin-III | Maintaining | Inhibits | Adenoma |
| | | | cellular | thrombin-induced | 1.2 (1.0-1.5) |
| | | | homeostasis | tumor growth and | |
| | | | | angiogenesis, | |
| | | | | impairing | |
| | | | | proliferation and | |
| | | | | migration of cancer | |
| | | | | cells | |
| 9 | APOE_212_NONGLY- | Apolipoprotein E | Involved in fat | Specific isoform | Adenoma |
| | COSYLATED | | metabolism, | associated with | 0.4 (0.1-0.9) |
| | | | binds to LDLR | decreased risk of | |
| | | | | colorectal neoplasia | |
| 12 | CERU_358_NONGLY- | Ceruloplasmin | Copper | Associated with | Adenoma |
| | COSYLATED | | carrying, iron | copper uptake, | 1.7 (1.2-2.4) |
| | | | metabolism | which plays a role | Hyperplastic |
| | | | | in tumor | 2.0 (1.1-3.5) |
| 11 | CERU_358_5402 | Ceruloplasmin | | angiogenesis | Adenoma |
| | | | | | 0.3 (0.1-0.9) |
| 13 | CFAI_70_5401 | Complement | Serine | Involved in animal | Serrated |
| | | Factor I | proteinase | models of early | 0.4 (0.2-0.8) |
| | | | essential for | colorectal adenomas | |
| | | | regulating | | |
| | | | complement | | |
| | | | cascade | | |
| 15 | FETUA_156_6513 | Alpha-2-HS- | Regulate the | Inhibits progression | Adenoma |
| | | glycoprotein | TGF-β1 | in tumors driven by | 1.4 (1.1-1.7) |
| | | | signaling | TGF-β²³ | |
| | | | pathway | | |
| 16 | FINC_542_6502 | Fibronectin | Extracellular | Involved in CRC | Serrated |
| | | | matrix | signaling pathways | 0.8 (0.6-0.9) |
| | | | glycoprotein | and tumorigenesis | |
| | | | that bind to | | |
| | | | integrins | | |
| 20 | HPT_241_6512 | Haptoglobin | Neutralize and | Associated with | Adenoma |
| | | | clears free | detecting tumor | 1.3 (1.1-1.5) |
| | | | heme³⁹ | and inflammatory | |
| | | | | responses during | |
| | | | | CRC angiogenesis | |
| 24 | IC1_253_5412 | Plasma | Protease | Gene expression | Adenoma |
| | | protease | inhibitor | down regulated in | 1.8 (1.4-2.3) |
| | | C1 inhibitor | involved in the | Familial | |
| | | | inhibition of the | adenomatous | |
| | | | complement | polyposis | |
| | | | system⁴⁰ | | |
| 26 | IGG1_297_3410 | Immunoglobulin | Control | Associated with | Adenoma |
| | | heavy constant | infection and | CRC prognosis | 1.6 (1.2-2.1) |
| | | gamma 1 | activate the | | |
| | | | complement | | |
| | | | system⁴¹ | | |
| 27 | KLKB1_396_5401 | Plasma | Selective | Associated with | Hyperplastic |
| | | Kallikrein | breakdown of | colitis in a murine | 0.5 (0.3-0.9) |
| | | | amino acid | model | |
| | | | bonds, | | |
| | | | bradykinin | | |
| | | | release | | |

TABLE 10
List of 6 biomarkers associated with presence of polyps, adjusting for lifestyle factors.

| Seq | | | | |
|---|---|---|---|---|
| ID | Biomarker | | | |
| NO | protein group | Function | Role in colonic polyps | RR (CI) |
| 38 | AGP1 | Regulates every single | Specific glycoforms | AP 1.32 (1.01-1.69) |
| | (Alpha-1-acid | event related to | may be involved in | HP 1.55 (1.04-2.30) |
| | glycoprotein) | inflammation | polyps/CRC | |
| 44 | CERU_NONGLY- | Copper carrying, iron | Associated with | AP 1.49 (1.01-2.19) |
| | COSYLATED | metabolism | copper uptake, | |
| | (Ceruloplasmin) | | which plays a | |
| | | | role in tumor | |
| | | | angiogenesis | |
| 45 | CFAI | Serine proteinase | Involved in animal | SP 0.49 (0.25-0.96) |
| | (Complement | essential for regulating | models of early | |
| | Factor I) | complement cascade | colorectal adenomas | |
| 48 | FINC | Extracellular matrix | Involved in CRC | SP 0.8 (0.6-0.9) |
| | (Fibronectin) | glycoprotein that bind | signaling pathways | |
| | | to integrins | and tumorigenesis | |
| 51 | IC1 | Protease inhibitor | Gene expression | AP 1.69 (1.24-2.30) |
| | (Plasma | involved in the | down regulated in | |
| | protease C1 | inhibition of the | Familial adenomatous | |
| | inhibitor) | complement system | polyposis | |
| 53 | KLKB1 | Selective breakdown | Associated with | HP 0.49 (0.26-0.92) |
| | (Plasma | of amino acid bonds, | colitis in a | |
| | kallikrein) | bradykinin release | murine model | |

Polyp formation has been strongly associated with lifestyle and behavioral factors, and, as a host response to these colonic events, aberrant glycosylation changes occur in the circulating proteome. Using advanced LC-MS technology, allowing high resolution and high throughput glycoproteomic profiling, we demonstrated associations with different polyp histology. Further studies are warranted to quantify lifestyle and behavioral risk factors and their effect on the development on high-grade dysplastic polyps and colorectal cancer. Colorectal cancer (CRC) is the second most common cause of cancer related mortality in the US and nearly 150,000 new cases of CRC are diagnosed annually with 50,000 deaths. Approximately 80% of CRCs arise from adenomatous polyps, also called adenomas, which grow slowly and progress to dysplasia and then cancer over a period of approximately ten years. Screening for advanced adenomas (AAs) and CRC is important given the ability to identify premalignant lesions and early-stage malignancy nearly 2-3 years before cases with symptoms. In the US, 1 in 3, or 23 million eligible people are not screened for colon cancer. As a result, 60% of colon cancers are diagnosed at a late stage when the cancer has already metastasized consequently making the treatment process more challenging. Early identification and intervention can lead to better prognosis for a patient and reduce CRC associated morbidity and mortality.

### VIII. Exemplary Embodiments for Adenoma or Colorectal Cancer Diagnosis and Treatment

The present disclosure concerns embodiments for systems, methods, and compositions related to identification of adenoma or colorectal cancer (CRC), or risk thereof, in an individual. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately either identify that an individual has an advanced adenoma or CRC or that has a higher risk for adenoma or CRC over the general population based on the presence of one or more markers in sample(s) from the individual. The individual may or may not be at a higher risk for advanced adenoma or CRC based on one or more risk factors. An individual may be at risk for CRC based on family or personal history; age (e.g., 50 or older); having one or more genetic markers associated with CRC; having inflammatory bowel disease such as Crohn's disease or ulcerative colitis; having a genetic syndrome such as familial adenomatous polyposis (FAP) or hereditary non-polyposis colorectal cancer (Lynch syndrome); having lack of regular physical activity; having a diet low in fruits and vegetables; having a low-fiber and/or high-fat diet; being overweight or obese; high alcohol consumption, and/or tobacco use. An individual may be at risk for adenomas based on age, body weight, waist circumference, blood lipid, and/or blood glucose levels.

In various embodiments of the disclosure, an individual is in need of identifying whether or not they have adenoma or CRC, or a risk thereof. The individual may be subjected to measuring or testing for one or more markers encompassed herein as a matter of routine health maintenance or because of a specific concern, for example, such as the presence of one or more risk factors and/or one or more symptoms of adenoma or CRC. The individual may be in need of such identification based on any one of the risk factors noted above, or the individual may be in need of such identification based on having one or more symptoms of adenoma or CRC.

In some cases, the analysis of the sample of the individual as described herein is the sole test utilized for identifying adenoma or CRC, whereas in other cases a medical provider may utilize one or more other tests, such as ultrasound; magnetic resonance imaging; CT scan; biopsy; a combination thereof, colonoscopy, and so forth. In particular embodiments, measuring for one or more peptide structure markers as in Table 1 or Table 2 are utilized alone or in conjunction with one or more of these tests.

The systems, methods, and compositions encompassed herein are sufficiently specific to utilize markers that distinguish between control and advanced adenoma or CRC. In some embodiments, the markers are accurate regardless of the status of one or more characteristics of the individual: biological sex, sample source, sample collection, smoker status, or age, as examples.

In some embodiments, the individual is suspected of having advanced adenoma or CRC or is at risk for advanced adenoma or CRC and is in need of diagnosis thereof in addition to identification whether it is a particular stage of CRC. In various embodiments, the individual is known to have CRC and is in need of determining whether it is early stage CRC or late stage CRC, such as to determine a treatment regimen for the cancer. In specific embodiments, the same test that identifies whether an individual has CRC determines whether the CRC is early stage or late stage or a particular stage.

In various embodiments, the sample for analysis for advanced adenoma or CRC identification may be a solid or fluid from the individual, such as stool, peripheral blood, serum, and/or plasma from the individual. The present disclosure provides for measuring for one or more circulating glycoproteins, glycopeptides, or non-glycosylated peptides in stool, blood, serum, or plasma to diagnose or identify the presence of advanced adenoma or CRC and/or to identify early stage or late stage CRC in an individual. In various embodiments, the sample is measured for 1, 2, 3, 4, 5, 6, 7, 8, 9, 10, 11, 12, 13, 14, 15, 16, 17, 18, 19, 20, or all of the peptides of Table 1 or Table 2.

Embodiments of the disclosure include methods of classifying samples, including stool, peripheral blood, serum, or plasma samples, from an individual suspected of having, known to have, or at risk for having advanced adenoma or CRC by measuring from the sample for one or more glycopeptides and/or non-glycosylated peptides encompassed herein. The methods encompass whether or not advanced adenoma or CRC is identified in the individual. In some cases, the measuring identifies the individual as not having advanced adenoma or CRC or as having advanced adenoma or CRC. In various embodiments, in cases wherein the individual has one or more glycopeptides and/or non-glycosylated peptides of Table 1 or Table 2, or certain levels thereof compared to control or healthy individuals, the individual may be determined to have advanced adenoma or CRC. In various embodiments, in cases wherein the individual lacks the glycopeptides and/or non-glycosylated peptides of Table 1 or Table 2, or has certain levels thereof compared to control or healthy individuals, the individual may be determined not to have advanced adenoma or CRC. The measuring may identify the individual as having a particular stage of CRC, including at least early stage or late stage. In specific cases, the measuring comprises successive or concomitant steps of identifying that the individual has advanced adenoma or CRC and whether the individual has early stage or late stage CRC.

In various embodiments, an individual at risk for having advanced adenoma or CRC is subjected to methods of the disclosure to identify, or not, the presence of advanced adenoma or CRC. Such methods also measure for one or more glycopeptides and/or non-glycosylated peptides encompassed herein. In various embodiments, in cases wherein the individual has one or more glycopeptides and/or non-glycosylated peptides of Table 1 or Table 2, the individual may be determined to have advanced adenoma or CRC. In various embodiments, in cases wherein the individual lacks the glycopeptides and/or non-glycosylated peptides of Table 1 or Table 2, the individual may be determined not to have advanced adenoma or CRC and is not treated for advanced adenoma or CRC. The individual may be of any kind, although in specific cases individual at risk for having advanced adenomas and/or colorectal cancer has a family history or one or more other risk factors.

Embodiments of the disclosure include methods of predicting that an individual will have advanced adenoma or CRC, including early stage or late stage CRC, or identifying early stage or late stage CRC in an individual, by measuring for one or more glycopeptides or non-glycosylated peptides from Table 1 or Table 2 in one or more samples from the individual. The individual may be known to have advanced adenoma or CRC or may be suspected of having advanced adenoma or CRC In various embodiments, the sample is measured for 1, 2, 3, 4, 5, 6, 7, 8, 9, 10, 11, 12, 13, 14, 15, 16, 17, 18, 19, 20, or all of the peptides of Table 1 or Table 2.

In embodiments wherein the measuring identifies the individual as having CRC, the individual may be recommended to take action to treat the CRC, such as with at least one of radiation therapy, chemotherapy or drug therapy (Bevacizumab, evacizumab, Irinotecan Hydrochloride, Capecitabine, Cetuximab, Ramucirumab, Oxaliplatin, Cetuximab, 5-FU, Ipilimumab, Irinotecan Hydrochloride, Pembrolizumab, Leucovorin Calcium, Trifluridine and Tipiracil Hydrochloride, Nivolumab, Nivolumab, Oxaliplatin. Panitumumab, Pembrolizumab, Ramucirumab, Regorafenib, Regorafenib, Panitumumab, Ziv-Aflibercept), chemoradiotherapy, surgery, hormone therapy and/or a targeted drug therapy, as examples.

Embodiments of the disclosure include methods of treating advanced adenoma or CRC in a subject, the method comprising: receiving a biological sample from the subject; determining a quantity of at least 1 peptide structure identified in Table 1 or Table 2 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has advanced adenoma or CRC; and administering a therapeutically effective amount of the treatment for advanced adenoma or CRC. The treatment may be of any kind, including at least one or more of biopsy, radiation therapy, chemotherapy, chemoradiotherapy, surgery, or a targeted drug therapy. In specific embodiments, the method further comprises preparing the biological sample to form a prepared sample comprising a set of peptide structures; and inputting the prepared sample into the MRM-MS system using a liquid chromatography system. The method may also be further defined as determining a quantity of at least 1 peptide structure identified in Table 1 or Table 2 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has advanced adenoma or CRC; and administering a therapeutically effective amount of the treatment for advanced adenoma or CRC.

Certain embodiments of the disclosure encompass methods of designing a treatment for a subject diagnosed with advanced adenoma or CRC state, the method comprising: designing a therapeutic regimen for treating the subject in response to measuring that a biological sample obtained from the subject evidences the state using part or all of any method encompassed herein, including identifying one or more peptide structures of Table 1 or Table 2. Various embodiments include methods of planning a treatment for a subject diagnosed with an advanced adenoma or CRC state, the method comprising: generating a treatment plan for treating the subject in response to measuring that a biological sample obtained from the subject evidences the state using part or all of any method encompassed herein, including identifying one or more peptide structures of Table 1 or Table 2.

Embodiments of the disclosure include methods of treating a subject diagnosed with advanced adenoma or CRC state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 1 or Table 2.

In various embodiments, methods of treating a subject diagnosed with advanced adenoma or CRC state are encompassed herein, the method comprising: selecting a therapeutic or treatment to treat the subject based on determining that the subject is responsive to the therapeutic using any method encompassed herein, including that identifies one or more peptide structures of Table 1 or Table 2.

In various embodiments, methods are included for classifying a sample from an individual suspected of having, known to have, or at risk for advanced adenoma or CRC, comprising the step of measuring from the sample for one or more glycopeptides and/or non-glycosylated peptides in Table 1 or Table 2. In specific embodiments, the measuring identifies the individual as not having advanced adenoma or CRC or as having advanced adenoma or CRC. The measuring may identify the individual as having early stage or late stage CRC, in specific embodiments, and the detection of early stage malignancy is useful such that a treatment path may be determined as soon as possible. In certain embodiments, the measuring comprises successive or concomitant steps of identifying that the individual has advanced adenoma or CRC and/or that the individual has early stage or late-stage CRC. The individual may or may not be at risk for advanced adenoma or CRC. In specific cases, when the measuring identifies the individual as having advanced adenoma or CRC, the individual is administered an effective amount of at least one of biopsy, radiation therapy, chemotherapy, chemoradiotherapy, surgery, or a targeted drug therapy. In various embodiments, the sample is measured for 1, 2, 3, 4, 5, 6, 7, 8, 9, 10, 11, 12, 13, 14, 15, 16, 17, 18, 19, 20, or all of the glycopeptides and/or non-glycosylated peptides of Table 1 or Table 2.

Embodiments of the disclosure include methods of diagnosing advanced adenoma or CRC in an individual, comprising the step of identifying 1, 2, 3, 4, 5, 6, 7, 8, 9, 10, 11, 12, 13, 14, 15, 16, 17, 18, 19, 20, or all of the peptide structures identified in Table 1 or Table 2 from a sample from the individual.

In various embodiments, an individual is measured for 1, 2, 3, 4, 5, 6, 7, 8, 9, 10, 11, 12, 13, 14, 15, 16, 17, 18, 19, 20, or all of the peptide structures identified in Table 1 or Table 2 from a sample from the individual for the purpose of identification of advanced adenoma or CRC. In specific embodiments, when 1, 2, 3, 4, 5, 6, 7, 8, 9, 10, 11, 12, 13, 14, 15, 16, 17, 18, 19, 20, or all of the peptide structures identified in Table 1 or Table 2 are measured in a sample from the individual, the individual is determined either to have advanced adenoma, to have CRC, or to require further testing to definitively determine whether the individual has advanced adenoma or CRC. In specific cases, the individual is subject to further testing of any kind and is determined either to have advanced adenoma or CRC, based on the presence of cancerous cells in the sample, for an example. Such further testing may or may not include colonoscopy, biopsy, biomarker testing of the cells, blood tests, CT scan, MRI, or a combination thereof.

In various embodiments, the disclosure relates to a method of screening a subject to identify and quantify risk of advanced adenoma or CRC, and thereby identify subjects suitable for further invasive investigation such as a colonoscopy. The method measures for certain one or more glycosylated or aglycosylated peptides that are shown to correlate with advanced adenoma or CRC and involves assaying a biological sample from the subject for one or a combination of biomarkers selected from PS-1 to PS-35, where the one or combination of biomarkers is chosen such that their detection correlates to at least an increased risk over the general population of the subject being positive for advanced adenoma or CRC. Detection of one or all of the combination of biomarkers indicates that the subject should undergo at least colonoscopy. In doing so, if one or more polyps and/or lesions are detected they may be removed for further analysis.

Subjects for which the systems and methods and compositions of the present disclosure may be subjected to may follow recommendations of The American Cancer Society that people at average risk of CRC start regular screening at age 45. An individual at average risk is considered one who has not had a personal history of colorectal cancer or certain types of polyps; a family history of colorectal cancer; a personal history of inflammatory bowel disease (ulcerative colitis or Crohn's disease); a confirmed or suspected hereditary colorectal cancer syndrome, such as familial advanced adenomatous polyposis (FAP) or Lynch syndrome (hereditary non-polyposis colon cancer or HNPCC); or a personal history of getting radiation to the abdomen (belly) or pelvic area to treat a prior cancer. In some cases, the subject may also be subjected to a stool-based test that looks for signs of cancer in a person's stool or with a visual exam that looks at the colon and rectum.

Subjects who are in good health and with a life expectancy of more than 10 years may be subjected to systems, methods and compositions of the present disclosure through the age of 75. Subjects aged 76 through 85 may be subjected to the systems, methods, and compositions of the present disclosure based on the subject's preferences, life expectancy, overall health, and prior screening history.

### IX. Additional Considerations

A1. A method for diagnosing a subject with respect to an advanced adenoma (AA) or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences the advanced adenoma or CRC disease state based on at least one peptide structure selected from a group of peptide structures identified in Table 2;
wherein the group of peptide structures in Table 2 is associated with the advanced adenoma or CRC disease state; and
generating a diagnosis output based on the disease indicator.

A2. The method of example A1, wherein the disease indicator comprises a score.

A3. The method of example A2, wherein generating the diagnosis output comprises:
determining that the score falls above a selected threshold; and
generating the diagnosis output based on the score falling above the selected threshold, wherein the diagnosis output includes a positive diagnosis for the advanced adenoma or CRC disease state.

A4. The method of example A2, wherein generating the diagnosis output comprises:
determining that the score falls below a selected threshold; and
generating the diagnosis output based on the score falling below the selected threshold, wherein the diagnosis output includes a negative diagnosis for the advanced adenoma or CRC disease state.

A5. The method of example A3 or example A4, wherein the score comprises a support vector machine score and the selected threshold is 0.

A6. The method of example A3 or example A4, wherein the selected threshold falls within a range between −0.1 and +0.1.

A7. The method of any one of examples A1-A6, wherein analyzing the peptide structure data comprises:
analyzing the peptide structure data using a binary classification model.

A8. The method of any one of examples A1-A7, wherein the at least one peptide structure comprises a glycopeptide structure defined by a peptide sequence and a glycan structure linked to the peptide sequence at a linking site of the peptide sequence, as identified in Table 2, with the peptide sequence being one of SEQ ID NOS: 3-9, 12, 14-16, 18, 25-28, and 31-35 as defined in Table 4C.

A9. The method of any one of examples A1-A8, further comprising:
training the at least one supervised machine learning model using training data,
wherein the training data comprises a plurality of peptide structure profiles for a plurality of subjects and a plurality of subject diagnoses for the plurality of subjects.

A10. The method of any one of examples A1-A8, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and high-risk advanced adenoma disease state,
wherein the second disease state includes one of colonoscopy negative control or non-advanced adenoma disease state.

A11. The method of example A10,
wherein the high-risk advanced adenoma disease state corresponds to a subject including one of:
(a) a tubular adenomas or serrated lesions (except HP) with low-grade dysplasia measuring ≥1.5 cm,
(b) a conventional adenomas or serrated lesions with high-grade dysplasia of any size,
(c) a tubulovillous/villous polyps of any size, and
(d) a combination thereof,
wherein the colonoscopy negative control disease state corresponds to a subject who had a colonoscopy where no polyp, lesion, or abnormal tissue was found in the colon,
wherein the non-advanced adenoma disease state corresponds to a subject having an adenoma and that the subject does not have the advanced adenoma disease state, wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof.

A12. The method of any one of examples A1-A8, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and advanced adenoma disease state,
wherein the second disease state includes one of colonoscopy negative control or non-advanced adenoma disease state.

A13. The method of example A12,
wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof,
wherein the colonoscopy negative control disease state corresponds to a sample from a subject who had a colonoscopy where no polyp, lesions, or abnormal tissue was found in the colon during the colonoscopy,
wherein the non-advanced adenoma disease state corresponds to a subject having an adenoma and that the subject does not have the advanced adenoma disease state.

A14. The method of examples A9, further comprising:
performing a differential expression analysis using initial training data to compare a first portion of the plurality of subjects diagnosed with the positive diagnosis for the CRC or advanced adenoma disease state versus a second portion of the plurality of subjects having
the negative diagnosis for the advanced adenoma or CRC disease state; and identifying a training group of peptide structures based on the differential expression analysis for use as prognostic markers for the advanced adenoma or CRC disease state; and
forming the training data based on the training group of peptide structures identified.

A15. The method of any one of examples A1-A14, wherein the peptide structure data comprises at least one of a raw abundance, an adjusted raw abundance, a peptide concentration, a glycopeptide concentration, or a normalized concentration.

16. The method of any one of examples 1-15, wherein the peptide structure data comprises normalized concentration data, wherein the normalized concentration data is a function of at least one of peptide abundance data, corresponding internal standard abundance data, a spike-in concentration value, and a dilution factor.

A17. The method of any one of examples A1-A16, wherein the peptide structure data is generated using multiple reaction monitoring mass spectrometry (MRM-MS).

A18. The method of any one of examples A1-A17, further comprising:
creating a sample from the biological sample; and
preparing the sample using reduction, alkylation, and enzymatic digestion to form a prepared sample that includes a set of peptide structures.

A19. The method of example A18, further comprising:
generating the peptide structure data from the prepared sample using multiple reaction monitoring mass spectrometry (MRM-MS).

A20. The method of any one of examples A1-A19, wherein generating the diagnosis output comprises:
generating a report identifying that the biological sample evidences the advanced adenoma or CRC disease state.

A21. The method of any one of examples A1-A20, further comprising:
generating a treatment output based on at least one of the diagnosis output or the disease indicator.

A22. The method of example A21, wherein the treatment output comprises at least one of an identification of a treatment to treat the subject or a treatment plan.

A23. The method of example A22, wherein the treatment comprises at least one of radiation therapy, chemoradiotherapy, surgery, hormone therapy, or a targeted drug therapy.

A24. The method of any one of examples A1-A23, wherein the generating the diagnosis output comprises the CRC disease state.

A24. The method of any one of examples A1-A23, wherein the diagnosing the subject is with respect to the colorectal cancer (CRC) disease state.

A25. The method of any one of examples A1-A24, wherein the analyzing the peptide structure data using the at least one supervised machine learning model to generate the disease indicator that indicates whether the biological sample evidences the advanced adenoma or CRC disease state based on at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all of the peptide structures selected from a group of peptide structures identified in Table 2.

A26. The method of any one of examples A1-25, wherein the at least one peptide structure comprises a peptide sequence and a glycan structure, wherein the glycan structure is attached to a linking site position in the peptide sequence in accordance with Table 2 and Table 4C.

A27. The method of example A62, wherein the glycan structure of the peptide sequence comprises a glycan structure GL number in accordance with Table 2, wherein the glycan structure comprises a composition in accordance with the glycan structure GL number, Table 6A, and Table 6B.

A28. The method of example A62, wherein the glycan structure of the peptide sequence comprises a glycan structure GL number in accordance with Table 2, wherein the glycan structure comprises a symbol structure in accordance with the glycan structure GL number, Table 6A, and Table 6B.

A29. The method of any one of examples A62-A64,
wherein a rightmost N-acetylgalactosamine of the glycan structure in Table 6B is attached to a linking site position in the peptide sequence in accordance with Table 2,
wherein a bottommost N-acetylglucosamine of the glycan structure in Table 6A is attached to a linking site position in the peptide sequence in accordance with Table 2.

B1. A composition comprising at least one peptide structure of Table 2.

C1. A composition comprising a peptide structure or a product ion, wherein:
the peptide structure or the product ion comprises an amino acid sequence having at least 90% sequence identity to any one of SEQ ID NOS: 3-9, 12, 14-16, 18, 25-28, and 31-35, corresponding to peptide structures in Table 2 and Table 4C; and
the product ion is selected as one from a group consisting of product ions identified in Table 3B including product ions falling within an identified m/z range.

D1. A composition comprising a glycopeptide structure selected as at least one peptide structure identified in Table 2, wherein:
the glycopeptide structure comprises:
an amino acid peptide sequence identified in Table 4C as corresponding to the glycopeptide structure; and
a glycan as corresponding to the glycopeptide structure in which the glycan is linked to a residue of the amino acid peptide sequence at a corresponding position identified in Table 2; and
wherein the glycan has a glycan composition.

D2. The composition of example D1, wherein the glycan composition is identified in Tables 6A and/or 6B.

D3. The composition of example D1, wherein:
the glycopeptide structure has a precursor ion having a charge identified in Table 3B as corresponding to the glycopeptide structure.

D4. The composition of example D1, wherein:
the glycopeptide structure has a precursor ion with an m/z ratio within +1.5 of the m/z ratio listed for the precursor ion in Table 3B as corresponding to the glycopeptide structure.

D5. The composition of example D1, wherein:
the glycopeptide structure has a precursor ion with an m/z ratio within +1.0 of the m/z ratio listed for the precursor ion in Table 3B as corresponding to the glycopeptide structure.

D6. The composition of example D1, wherein:
the glycopeptide structure has a precursor ion with an m/z ratio within +0.5 of the m/z ratio listed for the precursor ion in Table 3B as corresponding to the glycopeptide structure.

D7. The composition of example D1, wherein:
the glycopeptide structure has a product ion with an m/z ratio within +1.0 of the m/z ratio listed for the product ion in Table 3B as corresponding to the glycopeptide structure.

D8. The composition of example D1, wherein:
the glycopeptide structure has a product ion with an m/z ratio within +0.8 of the m/z ratio listed for the product ion in Table 3B as corresponding to the glycopeptide structure.

D9. The composition of example D1, wherein:
the glycopeptide structure has a product ion with an m/z ratio within +0.5 of the m/z ratio listed for the product ion in Table 3B as corresponding to the glycopeptide structure.

D10. The composition of any one of examples D1-D9, wherein the glycopeptide structure has a monoisotopic mass identified in Table 2 as corresponding to the glycopeptide structure.

E1. A method of screening a subject for an advanced adenoma or CRC disease state, the method comprising
analyzing a peptide structure data using at least one supervised machine learning model to generate a disease indicator that indicates whether the biological sample evidences the advanced adenoma or CRC disease state based on at least one peptide structure selected from a group of peptide structures identified in Table 2, wherein peptide structure data corresponds to a biological sample obtained from the subject; and
outputting either a recommendation to perform a colonoscopy or to not perform the colonoscopy based on the disease indicator.

E2. The method of example E1, wherein the group of peptide structures in Table 2 is associated with the advanced adenoma or CRC disease state.

E3. The method of examples E1-E2, wherein the group of peptide structures is listed in Table 2.

E4. The method of examples E1-E3, wherein the subject is subjected to a colonoscopy when the recommendation to perform the colonoscopy is outputted.

E5. The method of examples E1-E4, wherein the subject does not have any symptoms of the advanced adenoma and CRC disease state.

E6. The method of examples E1-E5 further comprising: receiving peptide structure data corresponding to the biological sample obtained from the subject.

E7. The method of examples E1-E6, wherein the disease indicator comprises a score, wherein generating the diagnosis output comprises:
determining that the score falls above a selected threshold; and
generating the diagnosis output based on the score falling above the selected threshold, wherein the diagnosis output includes a positive diagnosis for the advanced adenoma or CRC disease state.

E8. The method of any one of examples E1-E7, wherein analyzing the peptide structure data comprises:
analyzing the peptide structure data using a binary classification model.

E9. The method of any one of examples E1-E8, wherein the at least one peptide structure comprises a glycopeptide structure defined by a peptide sequence and a glycan structure linked to the peptide sequence at a linking site of the peptide sequence, as identified in Table 2, with the peptide sequence being one of SEQ ID NOS: 3-9, 12, 14-16, 18, 25-28, and 31-35 as defined in Table 2 and Table 4C.

E10. The method of any one of examples E1-E9, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and high-risk advanced adenoma disease state,
wherein the second disease state includes one of colonoscopy negative control or non-advanced adenoma disease state.

E11. The method of example E10,
wherein the high-risk advanced adenoma disease state corresponds to a subject including one of:
(a) a tubular adenomas or serrated lesions (except HP) with low-grade dysplasia measuring ≥1.5 cm,
(b) a conventional adenomas or serrated lesions with high-grade dysplasia of any size,
(c) a tubulovillous/villous polyps of any size, and
(d) a combination thereof,
wherein the colonoscopy negative control disease state corresponds to a subject who had a colonoscopy where no polyp, lesion, or abnormal tissue was found in the colon,
wherein the non-advanced adenoma disease state corresponds to a subject having an adenoma and that the subject does not have the advanced adenoma disease state, wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof.

E12. The method of any one of examples E1-E9, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and advanced adenoma disease state,
wherein the second disease state includes one of colonoscopy negative control or non-advanced adenoma disease state.

E13. The method of example E12,
wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof.
wherein the colonoscopy negative control disease state corresponds to a sample from a subject who had a colonoscopy where no polyp, lesions, or abnormal tissue was found in the colon during the colonoscopy,
wherein the non-advanced adenoma disease state corresponds to a subject having an adenoma and that the subject does not have the advanced adenoma disease state.

E14. The method of any one of examples E1-E13, wherein the peptide structure data comprises at least one of a raw abundance, an adjusted raw abundance, a peptide concentration, a glycopeptide concentration, or a normalized concentration.

E15. The method of any one of examples E1-E14, wherein the peptide structure data comprises normalized concentration data, wherein the normalized concentration data is a function of at least one of peptide abundance data, corresponding internal standard abundance data, a spike-in concentration value, and a dilution factor.

E16. The method of any one of examples E1-E15, wherein the peptide structure data is generated using multiple reaction monitoring mass spectrometry (MRM-MS).

E17. The method of any one of examples E1-E16, further comprising:
creating a sample from the biological sample; and
preparing the sample using reduction, alkylation, and enzymatic digestion to form a prepared sample that includes a set of peptide structures.

E18. The method of example E17, further comprising:
generating the peptide structure data from the prepared sample using multiple reaction monitoring mass spectrometry (MRM-MS).

E19. The method of any one of examples E1-E18 wherein the recommendation is a report identifying that the biological sample evidences the advanced adenoma or CRC disease state.

E20. The method of any one of examples E1-E19, wherein the advanced adenoma disease state occurs when the subject includes:
(a) a polyp measuring ≥1 centimeter in the greatest dimension,
(b) a polyp of any size with high-grade dysplasia,
(c) a tubulovillous/villous polyp of any size, and
(d) a combination thereof.

E21. The method of any one of examples E1-E20, wherein the analyzing the peptide structure data using the at least one supervised machine learning model to generate the disease indicator that indicates whether the biological sample evidences the CRC disease state based on at least one peptide structure selected from a group of peptide structures identified in Table 2, wherein peptide structure data corresponds to a biological sample obtained from the subject.

E22. The method of any one of examples E1-E21, wherein the analyzing a peptide structure data using the at least one supervised machine learning model to generate the disease indicator that indicates whether the biological sample evidences the advanced adenoma or CRC disease state based on at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all of the peptide structures selected from a group of peptide structures identified in Table 2, wherein peptide structure data corresponds to a biological sample obtained from the subject.

E22. The method of any one of examples E1-E22, wherein the at least one peptide structure comprises a peptide sequence and a glycan structure, wherein the glycan structure is attached to a linking site position in the peptide sequence in accordance with Table 2 and Table 4C.

E23. The method of example E22, wherein the glycan structure of the peptide sequence comprises a glycan structure GL number in accordance with Table 2, wherein the glycan structure comprises a composition in accordance with the glycan structure GL number, Table 6A, and Table 6B.

E24. The method of example E22, wherein the glycan structure of the peptide sequence comprises a glycan structure GL number in accordance with Table 2, wherein the glycan structure comprises a symbol structure in accordance with the glycan structure GL number, Table 6A, and Table 6B.

E25. The method of any one of examples E22-E24,
wherein a rightmost N-acetylgalactosamine of the glycan structure in Table 6B is attached to a linking site position in the peptide sequence in accordance with Table 2,
wherein a bottommost N-acetylglucosamine of the glycan structure in Table 6A is attached to a linking site position in the peptide sequence in accordance with Table 2.

F1. A method for diagnosing a subject with colorectal cancer (CRC) comprising:
detecting a presence or amount of at least one peptide structure selected from a group of peptide structures identified in Table 8B in a biological sample obtained from the subject and thereby diagnosing the individual as having colorectal cancer or not having colorectal cancer based upon the presence or amount of the at least one peptide structure selected from the group of peptide structures identified in Table 8B.

F2. The method of example F1, wherein the at least one peptide structure comprises a fucosylated glycopeptide having a tri-antennary glycan structure.

F3. The method of example F1, wherein the at least one peptide structure comprises a fucosylated glycopeptide having a tetra-antennary glycan structure.

G1. A method for determining a relative risk for a presence of polyps in a subject, the method comprising:
detecting a presence or amount of at least one peptide structure selected from a group of peptide structures identified in Table 9 in a biological sample obtained from the subject and thereby determining the relative risk for the presence of polyps in the subject based upon the presence or amount of the at least one peptide structure selected from the group of peptide structures identified in Table 9.

G2. The method of example G1, wherein the at least one peptide structure selected from a group of peptide structures identified in Table 9 comprises a peptide sequence corresponding to SEQ ID NOS: 4, 12, 13, 16, 24, and 27.

G3. The method of any one of examples G1-G2, wherein the at least one peptide structure comprises a peptide sequence and a glycan structure, wherein the glycan structure is attached to a linking site position in the peptide sequence in accordance with Table 8B and Table 4E.

G4. The method of example G3, wherein the glycan structure of the peptide sequence comprises a glycan structure GL number in accordance with Table 8B, wherein the glycan structure comprises a composition in accordance with the glycan structure GL number, Table 6A, and Table 6B.

G5. The method of example G3 and/or G4, wherein the glycan structure of the peptide sequence comprises a glycan structure GL number in accordance with Table 8B, wherein the glycan structure comprises a symbol structure in accordance with the glycan structure GL number, Table 6A, and Table 6B.

G6. The method of any one of examples G3-G5,
wherein a rightmost N-acetylgalactosamine of the glycan structure in Table 6B is attached to a linking site position in the peptide sequence in accordance with Table 8B,
wherein a bottommost N-acetylglucosamine of the glycan structure in Table 6A is attached to a linking site position in the peptide sequence in accordance with Table 8B.

H1. A method for treating a subject with respect to an advanced adenoma (AA) or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences the advanced adenoma or CRC disease state based on at least one peptide structure selected from a group of peptide structures identified in Table 2;
wherein the group of peptide structures in Table 2 is associated with the advanced adenoma or CRC disease state;
generating a diagnosis output based on the disease indicator; and
administering a treatment based on at least one of the diagnosis output or the disease indicator.

H2. The method of example H1, wherein the treatment comprises at least one of radiation therapy, chemoradiotherapy, surgery, hormone therapy, or a targeted drug therapy.

I1. A method of detecting one or more multiple-reaction-monitoring (MRM) transitions, comprising:
obtaining, or having obtained, a biological sample from a patient, wherein the biological sample comprises one or more glycans or glycopeptides;
digesting and/or fragmenting a glycopeptide in the sample; and
detecting a MRM transition selected from the group consisting of transitions corresponding to SEQ ID NOS: 3-9, 12, 14-16, 18, 25-28, and 31-35 as defined in Table 2 and Table 4C.

I2. The method of example I1, wherein the fragmenting a glycopeptide in the sample occurs after introducing the sample, or a portion thereof, into the mass spectrometer.

I3. The method of any one of example I1 or I2, wherein the fragmenting a glycopeptide in the sample produces a precursor ion or a product ion in accordance with Table 3B.

I4. The method of any one of examples I1-I3, further comprising conducting tandem liquid chromatography-mass spectroscopy on the biological sample.

I5. The method of any one of examples I1-I4, wherein the one or more MRM transitions are detected as the transition of the precursor ion and the product ion, wherein the precursor ion and the product ion each have a m/z value in accordance with Table 3B.

I6. The method of any one of examples I1-I5, wherein the glycopeptide structure is defined by a peptide sequence and a glycan linked to the peptide sequence at a linking site of the peptide sequence, as identified in Table 2, with the peptide sequence being one of SEQ ID NOS: 3-9, 12, 14-16, 18, 25-28, and 31-35 as defined in Table 4C.

J1. A method for diagnosing a subject with respect to an advanced adenoma (AA) or colorectal cancer (CRC) disease state, the method comprising:
receiving protein structure data corresponding to a biological sample obtained from the subject;
analyzing the protein structure data using at least one supervised machine learning model to generate a disease indicator that indicates whether the biological sample evidences the advanced adenoma or CRC disease state based on at least one protein structure selected from a group of protein structures identified in Table 2;
wherein the group of protein structures in Table 2 is associated with the advanced adenoma or CRC disease state; and
generating a diagnosis output based on the disease indicator.

J2. The method of J1, wherein the at least one protein structure comprises a glycoprotein structure defined by a protein sequence and a glycan structure linked to the protein sequence at a linking site of the protein sequence, as identified in Table 2, with the protein sequence being one of SEQ ID NOS: 36-57, 101-102 as defined in Table 5.

J3. The method of any one of examples J1-J2, wherein the at least one protein structure comprises a protein sequence and a glycan structure, wherein the glycan structure is attached to a linking site position in the protein sequence in accordance with Table 2 and Table 5.

J4. The method of example J3, wherein the glycan structure of the protein sequence comprises a glycan structure GL number in accordance with Table 2, wherein the glycan structure comprises a composition in accordance with the glycan structure GL number, Table 6A, and Table 6B.

J5. The method of example J3, wherein the glycan structure of the protein sequence comprises a glycan structure GL number in accordance with Table 2, wherein the glycan structure comprises a symbol structure in accordance with the glycan structure GL number, Table 6A, and Table 6B.

A29. The method of any one of examples J3-J5,
wherein a rightmost N-acetylgalactosamine of the glycan structure in Table 6B is attached to a linking site position in the peptide sequence in accordance with Table 2,
wherein a bottommost N-acetylglucosamine of the glycan structure in Table 6A is attached to a linking site position in the peptide sequence in accordance with Table 2.

In regard to any of the examples, the CRC stage 1 disease state corresponds to a subject having a cancer that has grown through a muscularis mucosa a submucosa, and the cancer has not spread to nearby lymph nodes or to distant sites.

In regard to any of the examples, the CRC stage 2 disease state corresponds to a subject having a cancer that has grown through a muscularis mucosa a submucosa, the cancer has also grown into the muscularis propria, and the cancer has not spread to nearby lymph nodes or to distant sites.

In regard to any of the examples, the biological sample can be in a tube that comprises an anticoagulant and a preserving agent. The method can further include isolating a plasma fraction from the tube to create a sample from the biological sample. The sample can be prepared using reduction, alkylation, and enzymatic digestion to form a prepared sample that includes a set of peptide structures.

In regard to any of the examples that use the biological sample and a tube including the anticoagulant and the preserving agent, the anticoagulant can include EDTA salt and the preserving agent can include imidazolidinyl urea.

In regard to any of the examples that use the biological sample and a tube including the anticoagulant and the preserving agent, the tube can further include glycine.

In regard to any of the examples that use the biological sample and a tube including the anticoagulant and the preserving agent, before the isolating the plasma fraction, the biological sample can contact the preserving agent for a period of time ranging from 24 hours to 7 days.

In regard to any of the examples, the biological sample can be in a tube that includes silica particles. The method further includes isolating a serum fraction from the tube to create a sample from the biological sample. The sample can be prepared using reduction, alkylation, and enzymatic digestion to form a prepared sample that includes a set of peptide structures.

In regard to any of the examples that use the biological sample and a tube including silica particles, the tube further includes a polyester gel configured to form a barrier between a serum fraction and blood cells during a centrifugation process.

In regard to any of the examples that use the biological sample and a tube including silica particles, the silica particles were spray-coated onto an inner surface of the tube.

In regard to any of the examples that use the biological sample and a tube including silica particles, the biological sample formed a clot in the tube before the isolating the serum fraction from the tube.

What is claimed:

1. A method for diagnosing a subject with respect to an advanced adenoma (AA) or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences the advanced adenoma or CRC disease state based on at least one peptide structure selected from a group of peptide structures identified in Table 2;
wherein the group of peptide structures in Table 2 is associated with the advanced adenoma or CRC disease state; and
generating a diagnosis output based on the disease indicator.

2. The method of claim 1, wherein the disease indicator comprises a score, wherein generating the diagnosis output comprises:
determining that the score falls above a selected threshold; and
generating the diagnosis output based on the score falling above the selected threshold, wherein the diagnosis output includes a positive diagnosis for the advanced adenoma or CRC disease state.

3. The method of claim 1, wherein the disease indicator comprises a score, wherein generating the diagnosis output comprises:
determining that the score falls below a selected threshold; and
generating the diagnosis output based on the score falling below the selected threshold, wherein the diagnosis output includes a negative diagnosis for the advanced adenoma or CRC disease state.

4. The method of claim 1, wherein the at least one peptide structure comprises a glycopeptide structure defined by a peptide sequence and a glycan structure linked to the peptide sequence at a linking site of the peptide sequence, as identified in Table 2, with the peptide sequence being one of SEQ ID NOS: 3-9, 12, 14-16, 18, 25-28, and 31-35 as defined in Table 4C.

5. The method claim 1, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and high-risk advanced adenoma disease state,
wherein the second disease state includes one of colonoscopy negative control or non-advanced adenoma disease state.

6. The method of claim 5,
wherein the high-risk advanced adenoma disease state corresponds to a subject including one of:
(a) a tubular adenomas or serrated lesions (except HP) with low-grade dysplasia measuring ≥1.5 cm,
(b) a conventional adenomas or serrated lesions with high-grade dysplasia of any size,
(c) a tubulovillous/villous polyps of any size, and
(d) a combination thereof,
wherein the colonoscopy negative control disease state corresponds to a subject who had a colonoscopy where no polyp, lesion, or abnormal tissue was found in the colon,
wherein the non-advanced adenoma disease state corresponds to a subject having an adenoma and that the subject does not have the advanced adenoma disease state, wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof.

7. The method of claim 1, wherein the peptide structure data comprises at least one of a raw abundance, an adjusted raw abundance, a peptide concentration, a glycopeptide concentration, or a normalized concentration.

8. The method of claim 7, wherein the peptide structure data comprises normalized concentration data, wherein the normalized concentration data is a function of at least one of peptide abundance data, corresponding internal standard abundance data, a spike-in concentration value, and a dilution factor.

9. The method of claim 1, further comprising:
creating a sample from the biological sample;
preparing the sample using reduction, alkylation, and enzymatic digestion to form a prepared sample that includes a set of peptide structures; and
generating the peptide structure data from the prepared sample using multiple reaction monitoring mass spectrometry (MRM-MS).

10. The method of claim 1, wherein the at least one peptide structure comprises a peptide sequence and a glycan structure, wherein the glycan structure is attached to a linking site position in the peptide sequence in accordance with Table 2 and Table 4C.

11. The method of claim 10, wherein the glycan structure of the peptide sequence comprises a glycan structure GL number in accordance with Table 2, wherein the glycan structure comprises a composition in accordance with the glycan structure GL number, Table 6A, and Table 6B.

12. The method of claim 10, wherein the glycan structure of the peptide sequence comprises a glycan structure GL number in accordance with Table 2, wherein the glycan structure comprises a symbol structure in accordance with the glycan structure GL number, Table 6A, and Table 6B.

13. The method of claim 12,
wherein a rightmost N-acetylgalactosamine of the glycan structure in Table 6B is attached to a linking site position in the peptide sequence in accordance with Table 2,
wherein a bottommost N-acetylglucosamine of the glycan structure in Table 6A is attached to a linking site position in the peptide sequence in accordance with Table 2.

14. A method of screening a subject for an advanced adenoma or CRC disease state, the method comprising
analyzing a peptide structure data using at least one supervised machine learning model to generate a disease indicator that indicates whether the biological sample evidences the advanced adenoma or CRC disease state based on at least one peptide structure selected from a group of peptide structures identified in Table 2, wherein peptide structure data corresponds to a biological sample obtained from the subject; and
outputting either a recommendation to perform a colonoscopy or to not perform the colonoscopy based on the disease indicator.

15. The method of claim 14, wherein the subject is subjected to a colonoscopy when the recommendation to perform the colonoscopy is outputted.

16. The method of claim 14, wherein the disease indicator comprises a score, wherein generating the diagnosis output comprises:
determining that the score falls above a selected threshold; and
generating the diagnosis output based on the score falling above the selected threshold, wherein the diagnosis output includes a positive diagnosis for the advanced adenoma or CRC disease state.

17. The method of claim 14, wherein the at least one peptide structure comprises a glycopeptide structure defined by a peptide sequence and a glycan structure linked to the peptide sequence at a linking site of the peptide sequence, as identified in Table 2, with the peptide sequence being one of SEQ ID NOS: 3-9, 12, 14-16, 18, 25-28, and 31-35 as defined in Table 2 and Table 4C.

18. The method of claim 14, further comprising:
training the at least one supervised machine learning model using training data,
wherein the training data comprises a plurality of peptide structure profiles for a plurality of subjects, the plurality of subject having either a first disease state or a second disease state,
wherein the first disease state includes one of CRC stage 1, CRC stage 2, and high-risk advanced adenoma disease state,
wherein the second disease state includes one of colonoscopy negative control or non-advanced adenoma disease state.

19. The method of claim 18,
wherein the high-risk advanced adenoma disease state corresponds to a subject including one of:
(a) a tubular adenomas or serrated lesions (except HP) with low-grade dysplasia measuring ≥1.5 cm,
(b) a conventional adenomas or serrated lesions with high-grade dysplasia of any size,
(c) a tubulovillous/villous polyps of any size, and
(d) a combination thereof,
wherein the colonoscopy negative control disease state corresponds to a subject who had a colonoscopy where no polyp, lesion, or abnormal tissue was found in the colon,
wherein the non-advanced adenoma disease state corresponds to a subject having an adenoma and that the subject does not have the advanced adenoma disease state, wherein the advanced adenoma disease state corresponds to a subject including one of:
(e) a polyp measuring ≥1 cm in the greatest dimension,
(f) a polyp of any size with high-grade dysplasia, and
(g) a tubulovillous/villous polyp of any size, and
(h) a combination thereof.

20. The method of claim 14, wherein the peptide structure data comprises at least one of a raw abundance, an adjusted raw abundance, a peptide concentration, a glycopeptide concentration, or a normalized concentration.

21. The method of any one of claim 20, wherein the peptide structure data comprises normalized concentration data, wherein the normalized concentration data is a function of at least one of peptide abundance data, corresponding internal standard abundance data, a spike-in concentration value, and a dilution factor.

22. The method of claim 14, further comprising:
creating a sample from the biological sample;
preparing the sample using reduction, alkylation, and enzymatic digestion to form a prepared sample that includes a set of peptide structures; and
generating the peptide structure data from the prepared sample using multiple reaction monitoring mass spectrometry (MRM-MS).

23. The method of claim 14 wherein the recommendation is a report identifying that the biological sample evidences the advanced adenoma or CRC disease state.

24. The method of claim 14, wherein the at least one peptide structure comprises a peptide sequence and a glycan structure, wherein the glycan structure is attached to a linking site position in the peptide sequence in accordance with Table 2 and Table 4C.

25. The method of claim 24, wherein the glycan structure of the peptide sequence comprises a glycan structure GL number in accordance with Table 2, wherein the glycan structure comprises a composition in accordance with the glycan structure GL number, Table 6A, and Table 6B.

26. The method of claim 24, wherein the glycan structure of the peptide sequence comprises a glycan structure GL number in accordance with Table 2, wherein the glycan structure comprises a symbol structure in accordance with the glycan structure GL number, Table 6A, and Table 6B.

27. The method of claim 26,
wherein a rightmost N-acetylgalactosamine of the glycan structure in Table 6B is attached to a linking site position in the peptide sequence in accordance with Table 2,
wherein a bottommost N-acetylglucosamine of the glycan structure in Table 6A is attached to a linking site position in the peptide sequence in accordance with Table 2.

28. A composition comprising a glycopeptide structure selected as at least one peptide structure identified in Table 2, wherein:
the glycopeptide structure comprises:
an amino acid peptide sequence identified in Table 4C as corresponding to the glycopeptide structure; and
a glycan corresponding to the glycopeptide structure in which the glycan is linked to a residue of the amino acid peptide sequence at a corresponding position identified in Table 2; and
wherein the glycan has a glycan composition identified in Tables 6A and/or 6B.

29. The composition of claim 28, wherein:
the glycopeptide structure has a precursor ion with an m/z ratio within ±1.5 of the m/z ratio listed for the precursor ion in Table 3B as corresponding to the glycopeptide structure.

30. The composition of claim 29, wherein:
the glycopeptide structure has a product ion with an m/z ratio within ±1.0 of the m/z ratio listed for the product ion in Table 3B as corresponding to the glycopeptide structure.


SEQUENCE LISTING (103 sequences)

SEQ ID NO: 1 - protein, 16 aa, synthetic construct
  CARBOHYD 4: note = N is linked to the N-linked glycan structure Hex(5)HexNAc(4)Fuc(0)NeuAc(1)
YLGNATAIFFLPDEGK

SEQ ID NO: 2 - protein, 16 aa, synthetic construct
  CARBOHYD 4: note = N is linked to the N-linked glycan structure Hex(5)HexNAc(4)Fuc(1)NeuAc(2)
YLGNATAIFFLPDEGK

SEQ ID NO: 3 - protein, 10 aa, synthetic construct
  CARBOHYD 6: note = N is linked to the N-linked glycan structure Hex(5)HexNAc(4)Fuc(0)NeuAc(2)
DIENFNSTQK

SEQ ID NO: 4 - protein, 15 aa, synthetic construct
  CARBOHYD 7: note = N is linked to the N-linked glycan structure Hex(7)HexNAc(6)Fuc(1)NeuAc(3)
QDQCIYNTTYLNVQR

SEQ ID NO: 5 - protein, 6 aa, synthetic construct
  CARBOHYD 5: note = N is linked to the N-linked glycan structure Hex(6)HexNAc(5)Fuc(0)NeuAc(3)
NEEYNK

SEQ ID NO: 6 - protein, 21 aa, synthetic construct
  CARBOHYD 12: note = N is linked to the N-linked glycan structure Hex(5)HexNAc(4)Fuc(0)NeuAc(1)
VYIHPFHLVIHNESTCEQLAK

SEQ ID NO: 7 - protein, 16 aa, synthetic construct
  CARBOHYD 5: note = N is linked to the N-linked glycan structure Hex(5)HexNAc(4)Fuc(0)NeuAc(2)
LGACNDTLQQLMEVFK

SEQ ID NO: 8 - protein, 19 aa, synthetic construct
  CARBOHYD 5: note = N is linked to the N-linked glycan structure Hex(5)HexNAc(4)Fuc(1)NeuAc(2)
SLTFNETYQDISELVYGAK

SEQ ID NO: 9 - protein, 15 aa, synthetic construct
AATVGSLAGQPLQER

SEQ ID NO: 10 - protein, 11 aa, synthetic construct
LGNWSAMPSCK

SEQ ID NO: 11 - protein, 14 aa, synthetic construct
  CARBOHYD 13: note = N is linked to the N-linked glycan structure Hex(5)HexNAc(4)Fuc(0)NeuAc(2)
AGLQAFFQVQECNK

SEQ ID NO: 12 - protein, 14 aa, synthetic construct
AGLQAFFQVQECNK

SEQ ID NO: 13 - protein, 10 aa, synthetic construct
  CARBOHYD 1: note = N is linked to the N-linked glycan structure Hex(5)HexNAc(4)Fuc(0)NeuAc(1)
NGTAVCATNR

SEQ ID NO: 14 - protein, 21 aa, synthetic construct
  CARBOHYD 12: note = N is linked to the N-linked glycan structure Hex(6)HexNAc(2)Fuc(0)NeuAc(0)
TVLTPATNHMGNVTFTIPANR

SEQ ID NO: 15 - protein, 15 aa, synthetic construct
  CARBOHYD 12: note = N is linked to the N-linked glycan structure Hex(6)HexNAc(5)Fuc(1)NeuAc(3)
VCQDCPLLAPLNDTR

SEQ ID NO: 16 - protein, 16 aa, synthetic construct
  CARBOHYD 8: note = N is linked to the N-linked glycan structure Hex(6)HexNAc(5)Fuc(0)NeuAc(2)
HEEGHMLNCTCFGQGR

SEQ ID NO: 17 - protein, 16 aa, synthetic construct
  CARBOHYD 7: note = N is linked to the N-linked glycan structure Hex(5)HexNAc(4)Fuc(0)NeuAc(2)
ALPQPQNVTSLLGCTH

SEQ ID NO: 18 - protein, 22 aa, synthetic construct
  CARBOHYD 15: note = N is linked to the N-linked glycan structure Hex(5)HexNAc(4)Fuc(0)NeuAc(2)
CSDGWSFDATTLDDNGTMLFFK

SEQ ID NO: 19 - protein, 16 aa, synthetic construct
  CARBOHYD 6: note = N is linked to the N-linked glycan structure Hex(5)HexNAc(4)Fuc(0)NeuAc(2)
VVLHPNYSQVDIGLIK

SEQ ID NO: 20 - protein, 16 aa, synthetic construct
  CARBOHYD 6: note = N is linked to the N-linked glycan structure Hex(6)HexNAc(5)Fuc(1)NeuAc(2)
VVLHPNYSQVDIGLIK

SEQ ID NO: 21 - protein, 24 aa, synthetic construct
  CARBOHYD 6: note = N is linked to the N-linked glycan structure Hex(7)HexNAc(6)Fuc(0)NeuAc(2)
MVSHHNLTTGATLINEQWLLTTAK

SEQ ID NO: 22 - protein, 8 aa, synthetic construct
  CARBOHYD 5: note = N is linked to the N-linked glycan structure Hex(5)HexNAc(4)Fuc(0)NeuAc(2)
DTFVNASR

SEQ ID NO: 23 - protein, 8 aa, synthetic construct
  CARBOHYD 6: note = A is linked to the N-linked glycan structure Hex(5)HexNAc(4)Fuc(1)NeuAc(1)
DTFVNASR

SEQ ID NO: 24 - protein, 19 aa, synthetic construct
  CARBOHYD 4: note = N is linked to the N-linked glycan structure Hex(5)HexNAc(4)Fuc(1)NeuAc(2)
VLSNNSDANLELINTWVAK

SEQ ID NO: 25 - protein, 9 aa, synthetic construct
  CARBOHYD 5: note = N is linked to the O-linked glycan structure Hex(3)HexNAc(3)Fuc(1)NeuAc(0)
EEQYNSTYR

SEQ ID NO: 26 - protein, 9 aa, synthetic construct
  CARBOHYD 5: note = N is linked to the N-linked glycan structure Hex(3)HexNAc(4)Fuc(1)NeuAc(0)
EEQYNSTYR

SEQ ID NO: 27 - protein, 21 aa, synthetic construct
  CARBOHYD 6: note = N is linked to the N-linked glycan structure Hex(5)HexNAc(4)Fuc(0)NeuAc(1)
IVGGTNSSWGEWPWQVSLQVK

SEQ ID NO: 28 - protein, 10 aa, synthetic construct
  CARBOHYD 1: note = N is linked to the N-linked glycan structure Hex(5)HexNAc(4)Fuc(0)NeuAc(2)
NFTENDLLVR

SEQ ID NO: 29 - protein, 21 aa, synthetic construct
  CARBOHYD 9: note = N is linked to the N-linked glycan structure Hex(5)HexNAc(4)Fuc(0)NeuAc(2)
QQQHLFGSNVTDCSGNFCLFR

SEQ ID NO: 30 - protein, 21 aa, synthetic construct
  CARBOHYD 9: note = N is linked to the N-linked glycan structure Hex(5)HexNAc(4)Fuc(0)NeuAc(2)
QQQHLFGSNVTDCSGNFCLFR

SEQ ID NO: 31 - protein, 8 aa, synthetic construct
  CARBOHYD 1: note = N is linked to the N-linked glycan structure Hex(5)HexNAc(4)Fuc(0)NeuAc(2)
NGSLFAFR

SEQ ID NO: 32 - protein, 20 aa, synthetic construct
  CARBOHYD 2: note = N is linked to the N-linked glycan structure Hex(7)HexNAc(6)Fuc(2)NeuAc(4)
FNLTETSEAEIHQSFQHLLR

SEQ ID NO: 33 - protein, 20 aa, synthetic construct
  CARBOHYD 2: note = N is linked to the N-linked glycan structure Hex(5)HexNAc(4)Fuc(0)NeuAc(2)
ENLTAPGSDSAVFFEQGTTR

SEQ ID NO: 34 - protein, 16 aa, synthetic construct
  CARBOHYD 6: note = N is linked to the N-linked glycan structure Hex(6)HexNAc(5)Fuc(1)NeuAc(3)
VVLHPNYSQVDIGLIK

SEQ ID NO: 35 - protein, 9 aa, synthetic construct
  CARBOHYD 5: note = N is linked to the N-linked glycan structure Hex(3)HexNAc(5)Fuc(1)NeuAc(0)
EEQYNSTYR

SEQ ID NO: 36 - protein, 418 aa, synthetic construct
  CARBOHYD 271: note = N is linked to the N-linked glycan structure Hex(5)HexNAc(4)Fuc(0)NeuAc(1)
  CARBOHYD 271: note = N is linked to the N-linked glycan structure Hex(5)HexNAc(4)Fuc(1)NeuAc(2)
  CARBOHYD 107: note = N is linked to the N-linked glycan structure Hex(6)HexNAc(5)Fuc(1)NeuAc(2)
  CARBOHYD 107: note = N is linked to the N-linked glycan structure Hex(6)HexNAc(5)Fuc(1)NeuAc(3)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 37 - protein, 599 aa, synthetic construct
  CARBOHYD 33: note = N is linked to the N-linked glycan structure Hex(5)HexNAc(4)Fuc(0)NeuAc(2)
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 38 - protein, 201 aa, synthetic construct
  CARBOHYD 56: note = N is linked to the N-linked glycan structure Hex(6)HexNAc(5)Fuc(0)NeuAc(3)
  CARBOHYD 103: note = N is linked to the N-linked glycan structure Hex(6)HexNAc(5)Fuc(1)NeuAc(3)
  CARBOHYD 93: note = N is linked to the N-linked glycan structure Hex(6)HexNAc(5)Fuc(1)NeuAc(3)
  CARBOHYD 56: note = N is linked to the N-linked glycan structure Hex(6)HexNAc(5)Fuc(1)NeuAc(3)
  CARBOHYD 72: note = N is linked to the N-linked glycan structure Hex(6)HexNAc(5)Fuc(1)NeuAc(3)
  CARBOHYD 103: note = N is linked to the N-linked glycan structure Hex(7)HexNAc(6)Fuc(1)NeuAc(2)
  CARBOHYD 93: note = N is linked to the N-linked glycan structure Hex(7)HexNAc(6)Fuc(1)NeuAc(2)
  CARBOHYD 93: note = N is linked to the N-linked glycan structure Hex(7)HexNAc(6)Fuc(1)NeuAc(3)
  CARBOHYD 72: note = N is linked to the N-linked glycan structure Hex(7)HexNAc(6)Fuc(1)NeuAc(3)
  CARBOHYD 103: note = N is linked to the N-linked glycan structure Hex(7)HexNAc(6)Fuc(1)NeuAc(4)
  CARBOHYD 93: note = N is linked to the N-linked glycan structure Hex(7)HexNAc(6)Fuc(1)NeuAc(4)
  CARBOHYD 72: note = N is linked to the N-linked glycan structure Hex(7)HexNAc(6)Fuc(1)NeuAc(4)
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 39 - protein, 201 aa, synthetic construct
  CARBOHYD 56: note = N is linked to the N-linked glycan structure Hex(6)HexNAc(5)Fuc(0)NeuAc(3)
  CARBOHYD 103: note = N is linked to the N-linked glycan structure Hex(6)HexNAc(5)Fuc(1)NeuAc(3)
  CARBOHYD 72: note = N is linked to the N-linked glycan structure Hex(7)HexNAc(6)Fuc(1)NeuAc(3)
  CARBOHYD 72: note = N is linked to the N-linked glycan structure Hex(7)HexNAc(6)Fuc(1)NeuAc(4)
  CARBOHYD 103: note = N is linked to the N-linked glycan structure Hex(7)HexNAc(6)Fuc(1)NeuAc(4)
  CARBOHYD 56: note = N is linked to the N-linked glycan structure Hex(6)HexNAc(5)Fuc(1)NeuAc(3)
  CARBOHYD 72: note = N is linked to the N-linked glycan structure Hex(6)HexNAc(5)Fuc(1)NeuAc(3)
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 40 - protein, 485 aa, synthetic construct
  CARBOHYD 47: note = N is linked to the N-linked glycan structure Hex(5)HexNAc(4)Fuc(0)NeuAc(1)
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 41 - protein, 464 aa, synthetic construct
  CARBOHYD 128: note = N is linked to the N-linked glycan structure Hex(5)HexNAc(4)Fuc(0)NeuAc(2)
  CARBOHYD 187: note = N is linked to the N-linked glycan structure Hex(5)HexNAc(4)Fuc(1)NeuAc(2)
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 42 - protein, 317 aa, synthetic construct [sequence omitted]

SEQ ID NO: 43 - protein, 345 aa, synthetic construct
  CARBOHYD 162: note = N is linked to the N-linked glycan structure Hex(6)HexNAc(5)Fuc(1)NeuAc(3)
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 44 - protein, 1065 aa, synthetic construct
  CARBOHYD 358: note = N is linked to the N-linked glycan structure Hex(5)HexNAc(4)Fuc(0)NeuAc(2)
  CARBOHYD 397: note = N is linked to the N-linked glycan structure Hex(5)HexNAc(4)Fuc(0)NeuAc(2)
  CARBOHYD 762: note = N is linked to the N-linked glycan structure Hex(6)HexNAc(5)Fuc(1)NeuAc(2)
  CARBOHYD 138: note = N is linked to the N-linked glycan structure Hex(6)HexNAc(5)Fuc(1)NeuAc(3)
  CARBOHYD 397: note = N is linked to the N-linked glycan structure Hex(6)HexNAc(5)Fuc(1)NeuAc(3)
  CARBOHYD 762: note = N is linked to the N-linked glycan structure Hex(6)HexNAc(5)Fuc(1)NeuAc(3)
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 45 - protein, 583 aa, synthetic construct
  CARBOHYD 70: note = N is linked to the N-linked glycan structure Hex(5)HexNAc(4)Fuc(0)NeuAc(1)
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 46 - protein, 1663 aa, synthetic construct
  CARBOHYD 85: note = N is linked to the N-linked glycan structure Hex(6)HexNAc(2)Fuc(0)NeuAc(0)
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 47 - protein, 367 aa, synthetic construct
  CARBOHYD 156: note = N is linked to the N-linked glycan structure Hex(6)HexNAc(5)Fuc(1)NeuAc(3)
  CARBOHYD 176: note = N is linked to the N-linked glycan structure Hex(6)HexNAc(5)Fuc(1)NeuAc(2)
  CARBOHYD 176: note = N is linked to the N-linked glycan structure Hex(6)HexNAc(5)Fuc(1)NeuAc(3)
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 48 - protein, 2477 aa, synthetic construct
  CARBOHYD 542: note = N is linked to the N-linked glycan structure Hex(6)HexNAc(5)Fuc(0)NeuAc(2)
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 49 - protein, 462 aa, synthetic construct
  CARBOHYD 453: note = N is linked to the N-linked glycan structure Hex(5)HexNAc(4)Fuc(0)NeuAc(2)
  CARBOHYD 64: note = N is linked to the N-linked glycan structure Hex(5)HexNAc(4)Fuc(0)NeuAc(2)
  CARBOHYD 187: note = N is linked to the N-linked glycan structure Hex(6)HexNAc(5)Fuc(1)NeuAc(3)
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 50 - protein, 406 aa, synthetic construct
  CARBOHYD 241: note = N is linked to the N-linked glycan structure Hex(5)HexNAc(4)Fuc(0)NeuAc(2)
  CARBOHYD 241: note = N is linked to the N-linked glycan structure Hex(6)HexNAc(5)Fuc(1)NeuAc(2)
  CARBOHYD 184: note = N is linked to the N-linked glycan structure Hex(7)HexNAc(6)Fuc(0)NeuAc(2)
  CARBOHYD 241: note = N is linked to the N-linked glycan structure Hex(6)HexNAc(5)Fuc(1)NeuAc(3)
  CARBOHYD 184: note = N is linked to the N-linked glycan structure Hex(6)HexNAc(5)Fuc(1)NeuAc(3)
  CARBOHYD 241: note = N is linked to the N-linked glycan structure Hex(7)HexNAc(6)Fuc(1)NeuAc(3)
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 51 - protein, 500 aa, synthetic construct
  CARBOHYD 238: note = N is linked to the N-linked glycan structure Hex(5)HexNAc(4)Fuc(0)NeuAc(2)
  CARBOHYD 238: note = N is linked to the N-linked glycan structure Hex(5)HexNAc(4)Fuc(1)NeuAc(1)
  CARBOHYD 253: note = N is linked to the N-linked glycan structure Hex(5)HexNAc(4)Fuc(1)NeuAc(2)
  CARBOHYD 253: note = N is linked to the N-linked glycan structure Hex(6)HexNAc(5)Fuc(1)NeuAc(3)
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 52 - protein, 399 aa, synthetic construct
  CARBOHYD 180: note = N is linked to the O-linked glycan structure Hex(3)HexNAc(3)Fuc(1)NeuAc(0)
  CARBOHYD 5: note = G is linked to the N-linked glycan structure Hex(3)HexNAc(4)Fuc(1)NeuAc(0)
  CARBOHYD 180: note = N is linked to the N-linked glycan structure Hex(3)HexNAc(5)Fuc(1)NeuAc(0)
  CARBOHYD 297: note = K is linked to the O-linked glycan structure Hex(3)HexNAc(3)Fuc(1)NeuAc(0)
  CARBOHYD 297: note = K is linked to the N-linked glycan structure Hex(3)HexNAc(4)Fuc(1)NeuAc(0)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 53 - protein, 638 aa, synthetic construct
  CARBOHYD 396: note = N is linked to the N-linked glycan structure Hex(5)HexNAc(4)Fuc(0)NeuAc(1)
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 54 - protein, 622 aa, synthetic construct
  CARBOHYD 416: note = N is linked to the N-linked glycan structure Hex(5)HexNAc(4)Fuc(0)NeuAc(2)
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 55 - protein, 698 aa, synthetic construct
  CARBOHYD 630: note = N is linked to the N-linked glycan structure Hex(5)HexNAc(4)Fuc(0)NeuAc(2)
  CARBOHYD 630: note = N is linked to the N-linked glycan structure Hex(6)HexNAc(5)Fuc(1)NeuAc(3)
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 56 - protein, 478 aa, synthetic construct
  CARBOHYD 169: note = N is linked to the N-linked glycan structure Hex(5)HexNAc(4)Fuc(0)NeuAc(2)
  CARBOHYD 242: note = N is linked to the N-linked glycan structure Hex(6)HexNAc(5)Fuc(1)NeuAc(3)
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 57 - protein, 423 aa, synthetic construct
  CARBOHYD 106: note = N is linked to the N-linked glycan structure Hex(7)HexNAc(6)Fuc(2)NeuAc(4)
  CARBOHYD 271: note = N is linked to the N-linked glycan structure Hex(6)HexNAc(5)Fuc(1)NeuAc(2)
  CARBOHYD 106: note = N is linked to the N-linked glycan structure Hex(7)HexNAc(6)Fuc(1)NeuAc(4)
  CARBOHYD 271: note = N is linked to the N-linked glycan structure Hex(6)HexNAc(5)Fuc(1)NeuAc(3)
  CARBOHYD 106: note = N is linked to the N-linked glycan structure Hex(7)HexNAc(6)Fuc(1)NeuAc(3)
  CARBOHYD 106: note = N is linked to the N-linked glycan structure Hex(6)HexNAc(5)Fuc(1)NeuAc(3)
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 58 - protein, 32 aa, synthetic construct
  CARBOHYD 14: note = N is linked to the N-linked glycan structure Hex(6)HexNAc(5)Fuc(1)NeuAc(2)
ADTHDEILEGLNFNLTEIPEAQIHEGFQELLR

SEQ ID NO: 59 - protein, 16 aa, synthetic construct
  CARBOHYD 4: note = N is linked to the N-linked glycan structure Hex(6)HexNAc(5)Fuc(1)NeuAc(2)
YTGNASALFILPDQDK

SEQ ID NO: 60 - protein, 17 aa, synthetic construct
  CARBOHYD 9: note = N is linked to the N-linked glycan structure Hex(6)HexNAc(5)Fuc(1)NeuAc(2)
ELHHLQEQNVSNAFLDK

SEQ ID NO: 61 - protein, 22 aa, synthetic construct
  CARBOHYD 11: note = N is linked to the N-linked glycan structure Hex(6)HexNAc(5)Fuc(1)NeuAc(2)
AALAAFNAQNNGSNFQLEEISR

SEQ ID NO: 62 - protein, 16 aa, synthetic construct
  CARBOHYD 6: note = N is linked to the N-linked glycan structure Hex(6)HexNAc(5)Fuc(1)NeuAc(2)
VVLHPNYSQVDIGLIK

SEQ ID NO: 63 - protein, 12 aa, synthetic construct
  CARBOHYD 9: note = N is linked to the N-linked glycan structure Hex(6)HexNAc(5)Fuc(1)NeuAc(2)
ITYSIVQTNCSK

SEQ ID NO: 64 - protein, 32 aa, synthetic construct; identical to SEQ ID NO: 58
  CARBOHYD 14: note = N is linked to the N-linked glycan structure Hex(6)HexNAc(5)Fuc(1)NeuAc(3)

SEQ ID NO: 65 - protein, 20 aa, synthetic construct
  CARBOHYD 2: note = N is linked to the N-linked glycan structure Hex(6)HexNAc(5)Fuc(1)NeuAc(3)
FNLTETSEAEIHQSFQHLLR

SEQ ID NO: 66 - protein, 16 aa, synthetic construct
  CARBOHYD 4: note = N is linked to the N-linked glycan structure Hex(6)HexNAc(5)Fuc(1)NeuAc(3)
YTGNASALFILPDQDK

SEQ ID NO: 67 - protein, 7 aa, synthetic construct
  CARBOHYD 2: note = N is linked to the N-linked glycan structure Hex(6)HexNAc(5)Fuc(1)NeuAc(3)
ENGTISR

SEQ ID NO: 68 - protein, 15 aa, synthetic construct
  CARBOHYD 7: note = N is linked to the N-linked glycan structure Hex(6)HexNAc(5)Fuc(1)NeuAc(3)
QDQCIYNTTYLNVQR

SEQ ID NO: 69 - protein, 6 aa, synthetic construct
  CARBOHYD 5: note = N is linked to the N-linked glycan structure Hex(6)HexNAc(5)Fuc(1)NeuAc(3)
NEEYNK

SEQ ID NO: 70 - protein, 16 aa, synthetic construct
  CARBOHYD 15: note = N is linked to the N-linked glycan structure Hex(6)HexNAc(5)Fuc(1)NeuAc(3)
SVQEIQATFFYFTPNK

SEQ ID NO: 71 - protein, 24 aa, synthetic construct
  CARBOHYD 15: note = N is linked to the N-linked glycan structure Hex(6)HexNAc(5)Fuc(1)NeuAc(3)
SVQEIQATFFYFTPNKTEDTIFLR

SEQ ID NO: 72 - protein, 7 aa, synthetic construct
  CARBOHYD 2: note = N is linked to the N-linked glycan structure Hex(6)HexNAc(5)Fuc(1)NeuAc(3)
ENGTVSR

SEQ ID NO: 73 - protein, 13 aa, synthetic construct
  CARBOHYD 8: note = N is linked to the N-linked glycan structure Hex(6)HexNAc(5)Fuc(1)NeuAc(3)
VYKPSAGNNSLYR

SEQ ID NO: 74 - protein, 30 aa, synthetic construct
  CARBOHYD 15: note = N is linked to the N-linked glycan structure Hex(6)HexNAc(5)Fuc(1)NeuAc(3)
FSLLGHASISCTVENETIGVWRPSPPTCEK

SEQ ID NO: 75 - protein, 16 aa, synthetic construct
  CARBOHYD 10: note = N is linked to the N-linked glycan structure Hex(6)HexNAc(5)Fuc(1)NeuAc(3)
EHEGAIYPDNTTDFQR

SEQ ID NO: 76 - protein, 20 aa, synthetic construct
  CARBOHYD 2: note = N is linked to the N-linked glycan structure Hex(6)HexNAc(5)Fuc(1)NeuAc(3)
ENLTAPGSDSAVFFEQGTTR

SEQ ID NO: 77 - protein, 17 aa, synthetic construct
  CARBOHYD 9: note = N is linked to the N-linked glycan structure Hex(6)HexNAc(5)Fuc(1)NeuAc(3)
ELHHLQEQNVSNAFLDK

SEQ ID NO: 78 - protein, 15 aa, synthetic construct
  CARBOHYD 12: note = N is linked to the N-linked glycan structure Hex(6)HexNAc(5)Fuc(1)NeuAc(3)
VCQDCPLLAPLNDTR

SEQ ID NO: 79 - protein, 22 aa, synthetic construct
  CARBOHYD 11: note = N is linked to the N-linked glycan structure Hex(6)HexNAc(5)Fuc(1)NeuAc(3)
AALAAFNAQNNGSNFQLEEISR

SEQ ID NO: 80 - protein, 13 aa, synthetic construct
  CARBOHYD 7: note = N is linked to the N-linked glycan structure Hex(6)HexNAc(5)Fuc(1)NeuAc(3)
SWPAVGNCSSALR

SEQ ID NO: 81 - protein, 24 aa, synthetic construct
  CARBOHYD 6: note = N is linked to the N-linked glycan structure Hex(6)HexNAc(5)Fuc(1)NeuAc(3)
MVSHHNLTTGATLINEQWLLTTAK

SEQ ID NO: 82 - protein, 16 aa, synthetic construct
  CARBOHYD 6: note = N is linked to the N-linked glycan structure Hex(6)HexNAc(5)Fuc(1)NeuAc(3)
VVLHPNYSQVDIGLIK

SEQ ID NO: 83 - protein, 19 aa, synthetic construct
  CARBOHYD 4: note = N is linked to the N-linked glycan structure Hex(6)HexNAc(5)Fuc(1)NeuAc(3)
VLSNNSDANLELINTWVAK

SEQ ID NO: 84 - protein, 22 aa, synthetic construct
  CARBOHYD 8: note = N is linked to the N-linked glycan structure Hex(6)HexNAc(5)Fuc(1)NeuAc(3)
HGIQYFNNNTQHSSLFMLNEVK

SEQ ID NO: 85 - protein, 12 aa, synthetic construct
  CARBOHYD 9: note = N is linked to the N-linked glycan structure Hex(6)HexNAc(5)Fuc(1)NeuAc(3)
ITYSIVQTNCSK

SEQ ID NO: 86 - protein, 12 aa, synthetic construct
  CARBOHYD 6: note = N is linked to the N-linked glycan structure Hex(6)HexNAc(5)Fuc(1)NeuAc(3)
LNAENNATFYFK

SEQ ID NO: 87 - protein, 21 aa, synthetic construct
  CARBOHYD 9: note = N is linked to the N-linked glycan structure Hex(6)HexNAc(5)Fuc(1)NeuAc(3)
QQQHLFGSNVTDCSGNFCLFR

SEQ ID NO: 88 - protein, 27 aa, synthetic construct
  CARBOHYD 1: note = N is linked to the N-linked glycan structure Hex(6)HexNAc(5)Fuc(1)NeuAc(3)
NISDGFDGIPDNVDAALALPAHSYSGR

SEQ ID NO: 89 - protein, 7 aa, synthetic construct
  CARBOHYD 2: note = N is linked to the N-linked glycan structure Hex(7)HexNAc(6)Fuc(1)NeuAc(2)
ENGTISR

SEQ ID NO: 90 - protein, 15 aa, synthetic construct
  CARBOHYD 7: note = N is linked to the N-linked glycan structure Hex(7)HexNAc(6)Fuc(1)NeuAc(2)
QDQCIYNTTYLNVQR

SEQ ID NO: 91 - protein, 15 aa, synthetic construct
  CARBOHYD 7: note = N is linked to the N-linked glycan structure Hex(7)HexNAc(6)Fuc(1)NeuAc(3)
QDQCIYNTTYLNVQR

SEQ ID NO: 92 - protein, 16 aa, synthetic construct
  CARBOHYD 15: note = N is linked to the N-linked glycan structure Hex(7)HexNAc(6)Fuc(1)NeuAc(3)
SVQEIQATFFYFTPNK

SEQ ID NO: 93 - protein, 24 aa, synthetic construct
  CARBOHYD 15: note = N is linked to the N-linked glycan structure Hex(7)HexNAc(6)Fuc(1)NeuAc(3)
SVQEIQATFFYFTPNKTEDTIFLR

SEQ ID NO: 94 - protein, 16 aa, synthetic construct
  CARBOHYD 6: note = N is linked to the N-linked glycan structure Hex(7)HexNAc(6)Fuc(1)NeuAc(3)
VVLHPNYSQVDIGLIK

SEQ ID NO: 95 - protein, 20 aa, synthetic construct
  CARBOHYD 2: note = N is linked to the N-linked glycan structure Hex(7)HexNAc(6)Fuc(1)NeuAc(4)
FNLTETSEAEIHQSFQHLLR

SEQ ID NO: 96 - protein, 7 aa, synthetic construct
  CARBOHYD 2: note = N is linked to the N-linked glycan structure Hex(7)HexNAc(6)Fuc(1)NeuAc(4)
ENGTISR

SEQ ID NO: 97 - protein, 15 aa, synthetic construct
  CARBOHYD 7: note = N is linked to the N-linked glycan structure Hex(7)HexNAc(6)Fuc(1)NeuAc(4)
QDQCIYNTTYLNVQR

SEQ ID NO: 98 - protein, 16 aa, synthetic construct
  CARBOHYD 15: note = N is linked to the N-linked glycan structure Hex(7)HexNAc(6)Fuc(1)NeuAc(4)
SVQEIQATFFYFTPNK

SEQ ID NO: 99 - protein, 24 aa, synthetic construct
  CARBOHYD 15: note = N is linked to the N-linked glycan structure Hex(7)HexNAc(6)Fuc(1)NeuAc(4)
SVQEIQATFFYFTPNKTEDTIFLR

SEQ ID NO: 100 - protein, 7 aa, synthetic construct
  CARBOHYD 2: note = N is linked to the N-linked glycan structure Hex(7)HexNAc(6)Fuc(1)NeuAc(4)
ENGTVSR

SEQ ID NO: 101 - protein, 644 aa, synthetic construct
  CARBOHYD 205: note = N is linked to the N-linked glycan structure Hex(6)HexNAc(5)Fuc(1)NeuAc(2)
  CARBOHYD 169: note = N is linked to the N-linked glycan structure Hex(6)HexNAc(5)Fuc(1)NeuAc(3)
  CARBOHYD 205: note = N is linked to the N-linked glycan structure Hex(6)HexNAc(5)Fuc(1)NeuAc(3)
  CARBOHYD 294: note = N is linked to the N-linked glycan structure Hex(6)HexNAc(5)Fuc(1)NeuAc(3)
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 102 - protein, 597 aa, synthetic construct
  CARBOHYD 221: note = N is linked to the N-linked glycan structure Hex(6)HexNAc(5)Fuc(1)NeuAc(3)
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 103 - protein, 7 aa, synthetic construct
ECDMHYK